(19) United States
(12) Patent Application Publication
(10) Pub. No.: US 2022/0267281 A1
(43) Pub. Date: Aug. 25, 2022

(54) UNSATURATED HETEROCYCLOALKYL AND HETEROAROMATIC ACYL HYDRAZONE LINKERS, METHODS AND USES THEREOF

(71) Applicant: ONTARIO INSTITUTE FOR CANCER RESEARCH (OICR), Toronto (CA)

(72) Inventors: Ahmed MAMAI, Mississauga (CA); Anh My CHAU, Toronto (CA); David Uehling, Toronto (CA)

(21) Appl. No.: 17/618,248

(22) PCT Filed: Jun. 12, 2020
(86) PCT No.: PCT/CA2020/050811

§ 371 Date: Dec. 10, 2021

Related U.S. Application Data
(60) Provisional application No. 62/860,527, filed on Jun. 12, 2019.

(51) Int. Cl. C07D 239/34; C07D 213/64; C07D 215/40; C07D 237/14; C07D 241/18; C07D 241/44; C07D 249/12; C07D 401/12; C07D 403/12; C07D 498/18

(52) U.S. Cl. CPC C07D 239/34; C07D 213/64; C07D 215/40; C07D 237/14; C07D 498/18; C07D 241/44; C07D 249/12; C07D 401/12; C07D 403/12; C07D 241/18

(57) ABSTRACT

The present application is directed to compounds of Formula (I), (II), (III) or (IV) compositions comprising these compounds, methods for their preparation and their uses, for example, as acyl hydrazone linkers, which can link two chemical entities together for further use as medicaments and/or diagnostics.

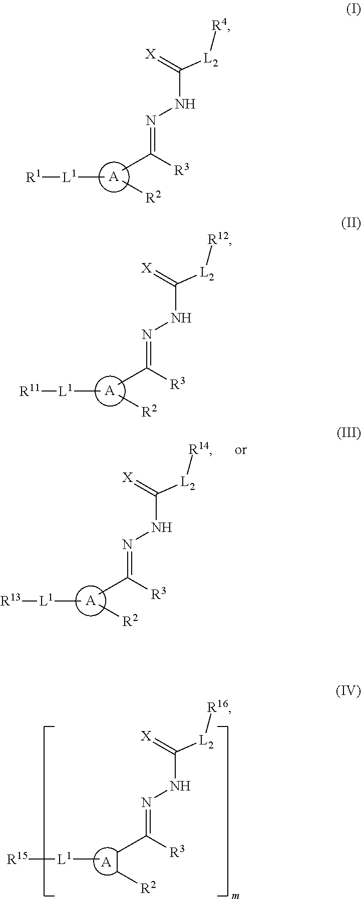

### RELATED APPLICATIONS

The present application claims the benefit of priority of co-pending U.S. provisional patent application No. 62/860,527 filed on Jun. 12, 2019, the contents of which are incorporated herein by reference in their entirety.

### FIELD

The present application relates to novel linker groups, to processes for their preparation, and for their use to link two chemical entities together, as well as to linked compounds comprising the linker groups and compositions comprising these linked compounds and to their use for example in the treatment or diagnosis of diseases and conditions, including, but not limited to, cancer.

### BACKGROUND

Chemotherapy, which targets rapidly dividing cancer cells, has proven to be one of the primary weapons in the arsenal to fight cancer. However, this approach is limited by the fact that it also affects healthy cells, typically resulting in moderate to severe side effects.¹⁻² Targeted therapies have the potential to greatly enhance the state of cancer therapeutics by selectively targeting cancerous cells while not harming healthy cells.³⁻⁷ Biologics such as monoclonal antibodies have emerged as options for cancer therapy due to their inherent specificity for cancer associated targets and their potential to have fewer off-target effects.⁸⁻¹⁰ In addition to carrying out the immune modulating functions of antibodies,¹¹ monoclonal antibodies have been used as a means of delivering cytotoxic drugs to cancer cells with high specificity, giving way to a type of therapeutic known as antibody-drug conjugates (ADC).¹²⁻¹⁶ ADCs have gained significant attention as a means of targeted delivery of cytotoxic agents to cancer cells. ADCs consist of a cytotoxic drug chemically attached to an antibody through a linker, and upon target cell binding and internalization, the drug is released. While this idea has limitless potential, its application is limited by the variable in vivo stability of the linker, which will lead to lower efficacy and higher off-target effects.

ADCs (FIG. 1) contain three distinct entities: (1) an antibody designed to target a tumour-associated antigen,¹⁷⁻¹⁸ (2) cytotoxic drugs,¹⁹⁻²¹ and (3) linkers that connect the drugs to the antibody.²²⁻²³ It is desirable that the ADC be stable, but upon antibody binding to the target cell and internalization, the drug is ideally released from the antibody to exert its actions.¹⁶ The efficacy and toxicity of ADCs depend heavily on the linker between the drug and the antibody and is affected by two factors: stability in plasma and drug to antibody ratio (DAR) and conjugation sites.²⁴ Currently, over 60 ADCs are in clinical trials, with 8 clinically approved: Adcetris™ (Brentuximab vedotin) targeting CD30 for anaplastic large cell lymphoma and Hodgkin's lymphoma approved in 2011, Kadcyla™ (Trastuzumab emtansine) was approved in 2013 for Her2⁺ metastatic breast cancer, Mylotarg™ (Gemtuzumab ozogamicin) targeting CD33 for acute myeloid leukemia, which was withdrawn from the market in 2010 due to excessive toxicity, was approved in 2017 under a different dosing regimen, Besponsa™ (Inotuzumab ozogamicin) was approved targeting CD22 for the treatment of refractory acute lymphoblastic leukemia²⁷⁻²⁸, Polivy™ (Polatuzumab vedotin) targeting CD79b was granted FDA approval for the treatment of diffuse large B-cell lymphomas in June 2019, Padcev™ (Enfortumab vedotin) targeting Nectin-4 was approved in December 2019 for the treatment of adult patients with locally advanced or metastatic urothelial cancers, Enhertu™ (fam-Trastuzumab deruxtecan) targeting Her2⁺ was approved in December 2019 as a treatment for unresectable or metastatic breast cancer following two or more prior anti-HER2 based regimens, and finally, Trodelvy™ (Sacituzumab govitecan), targeting Trop-2, was approved in April 2020 for the treatment of adult patients with metastatic triple-negative breast cancer who have received at least two prior therapies for metastatic disease.

There are currently two major classes of linkers used in ADCs: cleavable linkers such as acyl hydrazones,^{12, 27, 37-38} disulfides,^{20, 39-42} and peptides,^{22, 43-46} and non-cleavable linkers.^{22, 40-41} ADCs with acyl hydrazones as linkers are cleaved by the acidic environments of the lysosome. Disulfides and peptidic linkers are cleaved in thiol rich environments and by lysosomal peptidases but may have reduced potency, in part due to a greater difficulty of cleavage.^{37, 47} Noncleavable linkers will only break apart upon proteolytic degradation of the antibody post-internalization. While this linkage is very stable, internalization is essential, which may reduce its range of targets.⁴⁸ Taken together it is clear that the structure of the linker has a great impact on the stability, efficacy and safety of ADCs.

### SUMMARY

Heterocycloalkyl and heteroaromatic acyl hydrazone linkers have been prepared and conjugated to various drugs and antibodies to prepare ADCs.

Therefore, in one aspect, the present application includes a compound of Formula (I):

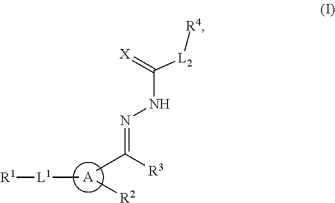

or a pharmaceutically acceptable salt and/or solvate thereof,

wherein:

Ring A is a 5 or 6 membered unsaturated heterocycloalkyl or 5 or 6 membered heteraromatic ring each comprising 1 to 4 heteroatoms selected from O, N and S, and Ring A is optionally substituted with one or more substituents independently selected from CN, NO₂, halo, C₁₋₆alkyl, C₁₋₆fluoroalkyl, ═O, OR⁵, SR⁵ and NR⁵R⁶;

R¹ and R⁴ are independently, a reactive functional group;

R² is absent or selected from H, CN, NO₂, halo, C₁₋₆alkyl, C₁₋₆fluoroalkyl, OR⁷, SR⁷ and NR⁷R⁸, and when present R² is ortho to

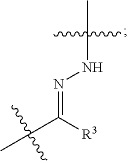

R³ is from H, C₁₋₄alkyl and C₁₋₄fluoroalkyl; or

R² and R³ are joined to form, together with the atoms therebetween, a 4 to 6 membered saturated or unsaturated ring, optionally containing one additional heteroatom selected from O, N and S, and optionally substituted with one or more substituents selected from C₁₋₆alkyl and C₁₋₆fluoroalkyl; and

X is selected from O, S and NR⁹;

R⁵, R⁶, R⁷, R⁸, and R⁹ are independently selected from H, C₁₋₆alkyl and C₁₋₆fluoroalkyl; and

L¹ and L² are independently a linker moiety.

In another aspect, the present application includes a compound of Formula (II):

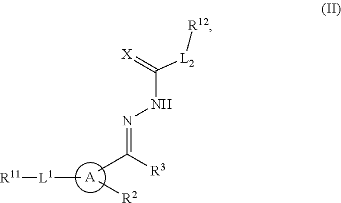

Ring A is a 5 or 6 membered unsaturated heterocycloalkyl or 5 or 6 membered heteraromatic ring each comprising 1 to 4 heteroatoms selected from O, N and S, and Ring A is optionally substituted with one or more substituents independently selected from CN, NO₂, halo, C₁₋₆alkyl, C₁₋₆fluoroalkyl, ═O, OR⁵, SR⁵ and NR⁵R⁶;

R² is absent or selected from H, CN, NO₂, halo, C₁₋₆alkyl, C₁₋₆fluoroalkyl, OR⁷, SR⁷ and NR⁷R⁸, and when present R² is ortho to

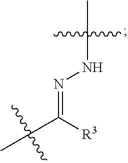

R³ is selected from H, C₁₋₄alkyl and C₁₋₄fluoroalkyl; or

R² and R³ are joined to form, together with the atoms therebetween, a 4 to 6 membered saturated or unsaturated ring, optionally containing one additional heteroatom selected from O, N and S, and optionally substituted with one or more substituents selected from C₁₋₆alkyl and C₁₋₆fluoroalkyl;

X is selected from O, S and NR⁹;

R⁵, R⁶, R⁷, R⁸, and R⁹ are independently selected from H, C₁₋₆alkyl and C₁₋₆fluoroalkyl;

R¹¹ and R¹² are different and are selected from compounds to be linked together; and L¹ and L² are independently a linker moiety.

In some embodiments, the compounds to be linked together are selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex and solid support.

In another aspect, the present application includes a compound of Formula (III):

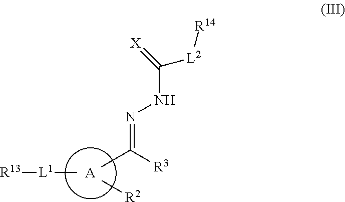

or a pharmaceutically acceptable salt and/or solvate thereof,

wherein one of R¹³ and R¹⁴ is a reactive functional group; and the other of R¹⁵ and R¹⁶ is a compound to be linked to another same or different compound; and

Ring A, R², R³, L¹, L² and X are as defined above.

In a further aspect, the present application includes an antibody-drug conjugate comprising an antibody covalently attached by a linker to one or more drugs, the conjugate having a Formula (IV):

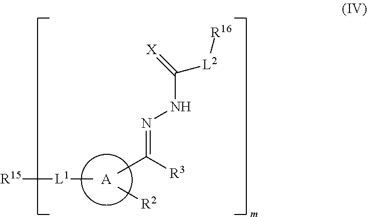

or a pharmaceutically acceptable salt and/or solvate thereof,

wherein

R¹⁵ is an antibody;

R¹⁶ is a drug;

Ring A, L¹, L², R² and R³ are as defined as above; and m is an integer from 1 to 20.

The present application includes a composition comprising one or more compounds of the application and a carrier. In an embodiment, the composition is a pharmaceutical composition comprising one or more compounds of Formula (II) or (III) and a pharmaceutically acceptable carrier.

The present application also includes a method of treating and/or diagnosing one or more diseases, disorders or conditions by administering an effective amount of one or more compounds of Formula (II), (III) or (IV), or a pharmaceutically acceptable salt and/or solvate thereof, to a subject in need thereof. In an embodiment of the present application, the disease, disorder or condition is cancer.

In another aspect, the present application includes a method of synthesizing one or more compounds of Formula (II) as defined above, or a pharmaceutically acceptable salt and/or solvate thereof, wherein the method comprises reacting one or more compounds of Formula (I) as defined above with a first compound, for example, selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex or solid support, and then a second, different compound, for example, selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex and solid support.

In another aspect the present application includes a method of preparing an ADC of Formula (IV) as defined above comprising:

(a) reacting a compound of Formula (I) with a drug to provide a Formula (I)-drug conjugate;

(b) reacting the Formula (I)-drug conjugate with an antibody to provide the ADC of Formula (IV); and optionally

(c) purifying the ADC of Formula (IV).

In another aspect, the present application includes a method of preparing an ADC of Formula (IV) as defined above comprising:

(a) reacting a compound of Formula (III) as defined above with an antibody to provide the ADC of Formula (IV); and optionally

(b) purifying the ADC of Formula (IV).

In another aspect of the present application is a use of one or more compounds Formula (II) and/or (IV), as defined above, or a pharmaceutically acceptable salt and/or solvate thereof, as a medicament and/or a diagnostic agent.

The unsaturated heterocylic and heteroaromatic hydrazone linkers of this present application have been demonstrated in an exemplary embodiment as linkers for ADCs. Therefore, compounds of Formula (II) and/or (IV) may be useful for treating diseases, disorders or conditions treatable by ADCs. In a further aspect, the present application includes a method of administering an antibody or a drug to a subject comprising administering a compound of Formula (II) and/or (IV), or a pharmaceutically acceptable salt and/or solvate thereof, to the subject.

In a further aspect of the application there is provided a use of one or more compounds of Formula (II) and/or (IV), as defined above, or a pharmaceutically acceptable salt and/or solvate thereof, to treat and/or diagnose cancer.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 is a schematic showing the general structure of an exemplary antibody-drug conjugate.

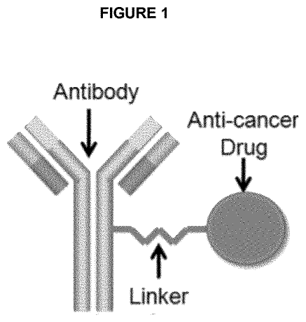

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

The term “and/or” as used herein means that the listed items are present, or used, individually or in combination. In effect, this term means that “at least one of” or “one or more” of the listed items is used or present. The term “and/or” with respect to salts and/or solvates thereof means that the compounds of the application exist as individual salts or hydrates, as well as a combination of, for example, a salt of a solvate of a compound of the application or a solvate of a salt of a compound of the application.

As used in the present application, the singular forms “a”, “an” and “the” include plural references unless the content clearly dictates otherwise. For example, an embodiment including “a compound” should be understood to present certain aspects with one compound or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The term “suitable” as used herein means that the selection of the particular compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The present description refers to a number of chemical terms and abbreviations used by those skilled in the art. Nevertheless, definitions of selected terms are provided for clarity and consistency.

The term “compound(s) of the application” or “compound(s) of the present application” and the like as used herein refers to a compound of Formula (I), (II), (III) or (IV) and/or salts and/or solvates thereof.

The term “composition of the application” or “composition of the present application” and the like as used herein refers to a composition comprising one or more compounds of the application.

The compounds of the present application may further exist in varying polymorphic forms and it is contemplated that any polymorphs, or mixtures thereof, which form are included within the scope of the present application.

The compounds of the present application may further be radiolabeled and accordingly all radiolabeled versions of the compounds of the application are included within the scope of the present application. There the compounds of the application also include those in which one or more radioactive atoms are incorporated within their structure.

The compounds of the present application also include those in which one or more hydrogen atoms are replaced with deuterium.

The term “linker moiety” as used herein refers to any molecular structure that joins two or more other molecular structures together.

The term “small molecule” as used herein refers to a molecule having a low molecular weight and with a size, for example, on the order of about 10 nm.

The term “reactive functional group” as used herein refers to a group of atoms or a single atom that will react with another group of atoms or a single atom (so called “complementary functional group”) to form a chemical interaction between the two groups or atoms.

The term “chemical interaction” as used herein refers to the formation of either a covalent or ionic bond between the reactive functional groups. The chemical interaction is one that is strong enough to append the acyl hydrazone linkers of the present application to compounds to be linked together.

The term “reacts with” as used herein generally means that there is a flow of electrons or a transfer of electrostatic charge resulting in the formation of a chemical interaction.

The term “conjugating” as used herein means to bind two molecules together via a chemical interaction.

The term “binding moiety” as used herein refers to any moiety that binds to a receptor or active site in a biological molecule. In an embodiment, the binding is specific binding, that is, the binding moiety will bind to one receptor or active site preferentially over other receptors or active sites.

The term “labelling agent” as used herein refers to any agent that is used for detection of molecules. Different types of labelling agents are known in the art depending on the form of detection to be used. For example, the labelling agent is selected from a radiolabel, a fluorescent label, a spin label, isotope label, a positron emission topography (PET) and a single-photon emission computer tomography label.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₆alkyl means an alkyl group having 1, 2, 3, 4, 5 or 6 carbon atoms. All alkyl groups are optionally fluorosubstituted unless otherwise indicated.

The term “alkylene” as used herein, whether it is used alone or as part of another group, means a straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₆alkylene means an alkylene group having 1, 2, 3, 4, 5 or 6 carbon atoms. All alkylene groups are optionally fluorosubstituted.

The term “alkenylene” as used herein, whether it is used alone or as part of another group, means a straight or branched chain, unsaturated alkylene group, that is, an unsaturated carbon chain that contains substituents on two of its ends and at least one double bond. The number of carbon atoms that are possible in the referenced alkenylene group are indicated by the prefix “Cₙ₁₋ₙ₂”. For example, the term C₂₋₆alkenylene means an alkenylene group having 2, 3, 4, 5 or 6 carbon atoms. All alkenylene groups are optionally fluorosubstituted, unless otherwise indicated.

The term “alkynylene” as used herein, whether it is used alone or as part of another group, means a straight or branched chain, unsaturated alkylene group, that is, an unsaturated carbon chain that contains substituents on two of its ends and at least one triple bond. The number of carbon atoms that are possible in the referenced alkynylene group are indicated by the prefix “Cₙ₁₋ₙ₂”. For example, the term C₂₋₆alkynylene means an alkynylene group having 2, 3, 4, 5 or 6 carbon atoms. All alkynylene groups are optionally fluorosubstituted, unless otherwise indicated.

The term “heterocycloalkyl” as used herein, whether it is used alone or as part of another group, refers to cyclic groups containing at least one non-aromatic ring in which one or more of the atoms are a heteroatom selected from O, S and N. Heterocycloalkyl groups are either saturated or unsaturated (i.e. contain one or more double bonds). When a heterocycloalkyl group contains the prefix “n1-n2-membered” or “n1 or n2-membered” this prefix indicates the number of atoms in the cyclic group, of which one or more are a heteroatom as defined above.

The term “unsaturated heterocycloalkyl” as used herein whether it is used alone or as part of another group, refers to cyclic groups containing at least one non-aromatic ring comprising one or more double bonds, and one or more of the atoms are a heteroatom selected from O, S and N. When a heterocycloalkyl group contains the prefix “n1-n2-membered” or “n1 or n2-membered” this prefix indicates the number of atoms in the cyclic group, of which one or more are a heteroatom as defined above.

The term “heteroaromatic” or “heteroaryl” as used herein, whether it is used alone or as part of another group, refers to cyclic groups containing at least one aromatic ring in which one or more of the atoms are a heteroatom selected from O, S and N. When a heteroaryl group contains the prefix “n1-n2-membered” or “n1 or n2-membered” this prefix indicates the number of atoms in the cyclic group, of which one or more are a heteroatom as defined above.

The term “heteroatom” as used herein, unless otherwise specified, refers to an atom other than carbon or hydrogen, and generally herein refers to O, S or N. Heteroatoms, such as N, may be substituted with additional substituents or hydrogen to fulfill valency requirements as would be known to those skilled in the art.

The term “optionally substituted” refers to groups, structures, or molecules that are either unsubstituted or are substituted with one or more substituents.

The term “fluorosubstituted” refers to the substitution of one or more, including all, hydrogens in a referenced group with fluorine.

The term “halo” or “halogen” as used herein, whether it is used along or as part of another group, refers to a halogen atom and includes fluoro, chloro, bromo and iodo.

The term “cell” as used herein refers to a single cell or a plurality of cells and includes a cell either in a cell culture or in a subject.

The term “subject” as used herein includes all members of the animal kingdom including mammals, and suitably refers to humans. Thus the methods of the present application are applicable to both human therapy and veterinary applications.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvate” as used herein means a compound, or a salt of a compound, wherein molecules of a suitable solvent are incorporated in the crystal lattice. A suitable solvent is physiologically tolerable at the dosage administered.

The term “MS” as used herein refers to mass spectrometry.

DCM as used herein refers to dichloromethane.

DCE as used herein refers to dichloroethane.

DIPEA as used herein refers to N,N-diisopropyl ethylamine

DMF as used herein refers to dimethylformamide.

THF as used herein refers to tetrahydrofuran.

DMSO as used herein refers to dimethylsulfoxide.

EtOAc as used herein refers to ethyl acetate.

MeOH as used herein refers to methanol.

HCl as used herein refers to hydrochloric acid.

TFA as used herein refers to trifluoroacetic acid.

mCPBA as used herein refers to meta-chloroperoxybenzoic acid.

RT as used herein refers to room temperature.

RB as used herein refers to a round bottom flask.

TBAF as used herein refers to tetra-n-butylammonium fluoride.

MW as used herein refers to molecular weight.

HPLC as used herein refers to high performance liquid chromatography.

LCMS as used herein refers to liquid chromatography-mass spectrometry.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3^{rd} Edition, 2003, Georg Thieme Verlag (The Americas).

The term “treating” or “treatment” as used herein and as is well understood in the art, means an approach for obtaining beneficial or desired results, including clinical results. In some embodiments, beneficial or desired clinical results may include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” may also mean prolonging survival as compared to expected survival if not receiving treatment. “Treating” and “treatment” as used herein may also include prophylactic treatment. For example, a subject with early cancer may be treated to prevent progression, or alternatively a subject in remission may be treated to prevent recurrence. Treatment methods comprise administering to a subject a therapeutically effective amount of one or more of the compounds and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

As used herein, the term “effective amount” or “therapeutically effective amount” means an amount of one or more compounds that is effective, at dosages and for periods of time necessary to achieve the desired result. For example in the context of a treatment for a disease, disorder of condition, an effective amount is an amount that, for example, increases said treatment compared to the treatment without administration of the one or more compounds.

The term “administered” as used herein means administration of a therapeutically effective amount of one or more compounds or compositions to a cell, tissue, organ or subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder.

The term “cancer” as used herein refers to cellular-proliferative disease states.

The term “antibody” as used herein refers to a full-length antibody molecule or an immunologically active portion of a full-length antibody molecule, i.e., a molecule that contains an antigen binding site that immunospecifically binds antigen of a target of interest or part thereof, such targets including but not limited to, cancer cells that produce specific identifiable antigens. The term “antibody” also refers to monoclonal antibodies, polyclonal antibodies, multispecific antibodies (e.g., bispecific antibodies), and antibody fragments. Antibodies may be murine, human humanized, chimeric, or derived from other species.

The term “monoclonal antibody” as used herein refers to an antibody obtained from a population of substantially homogenous antibodies, i.e., the individual antibodies comprising the population are identical except for possible naturally occurring mutations that may be present in minor amounts. Monoclonal antibodies are highly specific, being directed towards a single antigenic site. In contrast to polyclonal antibody preparations which include different antibodies directed against different determinants (epitopes), each monoclonal antibody is directed against a single determinant on the antigen. In addition to their specificity, the monoclonal antibodies are advantageous as they can be synthesized uncontaminated by other antibodies. The modifier “monoclonal” indicates the character of the antibody as being obtained from a substantially homogenous population of antibodies, and is not to be construed as requiring production of the antibody by any particular method.

The term “ErbB” as used herein is a receptor protein tyrosine kinase which belongs to the ErbB receptor family responsible for mediating cell growth, differentiation and survival. The ErbB receptor family includes four distinct members including epidermal growth factor receptor (EGFR, ErbB1, HER1), HER2 (ErbB2 or p185^{neu}), HER3 (ErbB3) and HER4 (ErbB4 or tyro2).

The terms “epidermal growth factor receptor” or “EGFR”, includes naturally occurring and mutant forms thereof (e.g., a deletion mutant EGFR).

The term “ErbB-expressing cancer” is a cancer characterized by comprising cells which have ErbB protein present at least at their cell surface. In an embodiment, the ErbB protein is the EGFR protein which is produced at sufficient levels at the surface of the cells such that an anti-EGFR antibody can bind thereto and have a therapeutic and/or diagnostic effect with respect to the cancer.

A “chemotherapeutic agent” or “anticancer agent” are terms that refer to a chemical compound useful in the treatment of a neoplastic disorder or cancer.

The term “drug” as used herein, is intended to refer to any compound or mixture of compounds which is capable of exerting an effective pharmacological effect.

The term DM1 as used herein refers to a compound of the formula

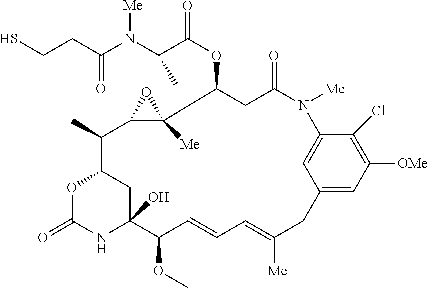 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2]

including pharmaceutically acceptable salts and/or solvates thereof. DM1 is also known as mertansine, and in some of its forms, emtansine.

The term “monomethyl auristatin E” or “MMAE” as used herein refers to a compound of the formula

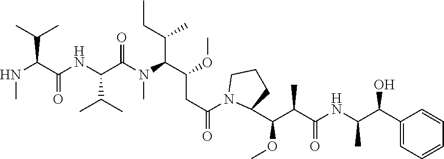 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

including pharmaceutically acceptable salts and/or solvates thereof.

### II. Compounds of the Application

The present application includes the design and optimization of acyl hydrazone linkers that can generally be used with a wide variety of molecular classes and tolerate many different functional groups.

Accordingly, the present application includes a compound of Formula (I):

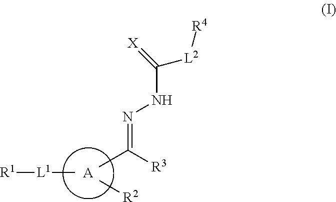

or a pharmaceutically acceptable salt and/or solvate thereof,

wherein:

Ring A is a 5 or 6 membered unsaturated heterocycloalkyl or a 5 or 6 membered heteraromatic ring each comprising 1 to 4 heteroatoms selected from O, N and S, and Ring A is optionally substituted with one or more substituents independently selected from CN, NO₂, halo, C₁₋₆alkyl, C₁₋₆fluoroalkyl, ═O, OR⁵, SR⁵ and NR⁵R⁶;

R¹ and R⁴ are independently a reactive functional group;

R² is absent or selected from H, CN, NO₂, halo, C₁₋₆alkyl, C₁₋₆fluoroalkyl, OR⁷, SR⁷ and NR⁷R⁸, and when present R² is ortho to Y R³

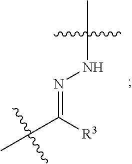

R³ is selected from H, C₁₋₄alkyl and C₁₋₄fluoroalkyl; or

R² and R³ are joined to form, together with the atoms therebetween, a 4 to 6 membered saturated or unsaturated ring, optionally containing one additional heteroatom selected from O, N and S, and optionally substituted with one or more substituents selected from C₁₋₆alkyl and C₁₋₆fluoroalkyl; X is selected from O, S and NR⁹;

R⁵, R⁶, R⁷, R⁸, and R⁹ are independently selected from H, C₁₋₆alkyl and C₁₋₆fluoroalkyl; and

L¹ and L² are independently a linker moiety.

In some embodiments, X is O.

In some embodiments, Ring A is a 5 or 6 membered heteroaromatic ring. In some embodiments, Ring A is selected from pyridinyl, pyrimidinyl, pyridazinyl, pyrazinyl, triazinyl, tetrazinyl, thienyl, furanyl, pyrrolyl, triazolyl, thiazolyl, oxazolyl and pyrazolyl. In some embodiments, Ring A is a 6 membered heteroaromatic ring. In some embodiments, Ring A is selected from pyridinyl, pyrimidinyl, pyrazinyl and pyridazinyl. In some embodiments, L¹ is located in the position para to

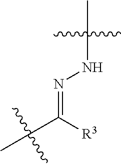

### on Ring A.

In some embodiments, Ring A is optionally substituted with one or more substituents independently selected from CN, NO₂, halo, C₁₋₆alkyl, C₁₋₆fluoroalkyl, OR⁵ and SR⁵. In some embodiments, Ring A is optionally substituted with one or three substituents are independently selected from CN, halo, C₁₋₆alkyl and C₁₋₆fluoroalkyl. In some embodiments, Ring A is optionally substituted with one or two substituents are independently selected from CH₃, CF₃, CH₂CH₃, CH₂CH₂F, CH₂CF₂H and CH₂CF₃.

In some embodiments, R² is absent. In some embodiments, R² is selected from H, CN, NO₂, halo, C₁₋₆alkyl, C₁₋₆fluoroalkyl, OR⁷ and SR⁷. In some embodiments, R² is selected from H, CN, halo, C₁₋₆alkyl and C₁₋₆fluoroalkyl. In some embodiments, R² is selected from H and CH₃. In some embodiments, R² is H.

In some embodiments, R³ is selected from H, CH₃, CF₃, CH₂CH₃, CH₂CH₂F, CH₂CF₂H and CH₂CF₃. In some embodiments, R³ is selected from H and CH₃. In some embodiments, R³ is CH₃.

In some embodiments, R² and R³ are joined to form, together with the atoms therebetween, a 5 to 6 membered saturated or unsaturated carbocyclic ring, optionally substituted with one or more substituents selected from C₁₋₆alkyl and C₁₋₆fluoroalkyl. In some embodiments, R² and R³ are joined to form a 6 membered saturated or unsaturated ring, optionally substituted with one or two substituents selected from C₁₋₆alkyl and C₁₋₆fluoroalkyl. In some embodiments, R² and R³ are joined to form a 6 membered unsaturated ring.

In some embodiments, R² and R³ are joined to form, together with the atoms therebetween, a 4 to 6 membered saturated or unsaturated ring, containing one additional heteroatom selected from O, N and S, and optionally substituted with one or more substituents selected from C₁₋₆alkyl and C₁₋₆fluoroalkyl.

In some embodiments, Ring A is a 5 or 6 membered unsaturated heterocycloalkyl ring. In some embodiments, Ring A is triazolyl and is Ring A is optionally substituted with one or three substituents independently selected from CN, NO₂, halo, C₁₋₆alkyl, C₁₋₆fluoroalkyl, ═O, OR⁵ and SR⁵, suitably one or two substituents independently selected from C₁₋₆alkyl, C₁₋₆fluoroalkyl and ═O, more suitably one or two substituents independently selected from CH₃, CF₃, CH₂HC₃, CH₂CH₂F, CH₂CF₂H, CH₂CF₃ and ═O.

In some embodiments, Ring A is triazolonyl. In some embodiments, Ring A is triazolonyl, and the compound of Formula (I) has the following structure:

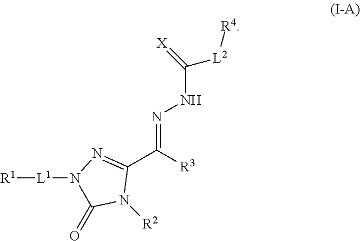

In some embodiments, R⁵, R⁶, R⁷, R⁸, and R⁹ are independently selected from H, C₁₋₄alkyl and C₁₋₄fluoroalkyl. In some embodiments, R⁵, R⁶, R⁷, R⁸, and R⁹ are independently selected from H and C₁₋₄alkyl. In some embodiments, R⁵ and R⁷ are independently H. In some embodiments, R⁵ and R⁷ are independently selected from methyl, ethyl, propyl, isopropyl, sec-butyl, n-butyl and t-butyl. In some embodiments, R⁵ and R⁷ are independently selected from H and methyl. In some embodiments, R⁶ and R⁸ are H.

In some embodiments, L¹ and L² independently comprise at least one ester, carbonate, carbamate or amide linkage although a person skilled in the art would appreciate that other linker moieties, such as ethers, sulfones, sulfoxides, thioethers, thioamides, thioesters and/or amines can additionally, or alternatively, be present. In some embodiments, L¹ and L² independently also comprise one or more C₁-C₂₀alkylene groups, C₂-C₂₀alkenylene groups and C₂-C₂₀alkynylene groups.

In some embodiments, L¹ and L² are independently selected from a direct bond, Z, R^{a}, Z—R^{a}, R^{a}—Z, R^{a}—Z—R^{b} and Z—R^{a}—Z^{a}, wherein Z and Z^{a} are independently selected from O, S, S(O), SO₂, NH, N(C₁₋₆alkyl), C(Q), C(Q)Y, YC(Q), YC(Q)Y^{a}, (C₁₋₆alkyleneY)ₚ and Y—(C₁₋₆alkyleneY)ₚ, wherein R^{a} and R^{b} are independently selected from C₁₋₁₀alkylene, C₂₋₁₀alkenylene and C₂₋₁₀alkynylene; Q, Y and Y^{a} are independently selected from O, S, NH and N(C₁₋₆alkyl); and p is selected from 1, 2, 3, 4, 5 and 6.

In some embodiments, R^{a} and R^{b} are independently selected from C₁₋₆alkylene, C₂₋₆alkenylene and C₂₋₆alkynylene. In some embodiments, R^{a} and R^{b} are independently selected from C₁₋₆alkylene.

In some embodiments, Q, Y and Y^{a} are independently selected from O, S, NH and N(CH₃).

In some embodiments Z and Z^{a} are independently selected from O, S, S(O), SO₂, NH, N(CH₃), C(O), C(O)NH, NHC(O), NHC(O)O, OC(O)O, NHC(O)NH, OC(O)NH, NHC(NH)NH, (C₁₋₆alkyleneO)ₚ and O—(C₁₋₆alkyleneO)ₚ. In some embodiments, Z and Z^{a} are independently selected from O, NH, C(O)NH and NHC(O).

In some embodiments L¹ is selected from OC(O)C₁₋₁₀alkyleneO, NHC(O)C₁₋₁₀alkyleneO, C₁₋₆alkyleneO, OC(O)C₁₋₁₀alkyleneNH, NHC(O)C₁₋₁₀alkyleneNH, C₁₋₆alkyleneNH, C(O)C₁₋₁₀alkyleneO and C(O)C₁₋₁₀alkyleneNH. In some embodiments L¹ is selected from OC(O)C₁₋₁₀alkyleneO, NHC(O)C₁₋₁₀alkyleneO, C₁₋₆alkyleneO, OC(O)C₁₋₁₀alkyleneNH, NHC(O)C₁₋₁₀alkyleneNH, C₁₋₆alkyleneNH, C(O)C₁₋₁₀alkyleneO, C(O)C₁₋₁₀alkyleneNH, NHC(O)C₁₋₁₀alkyleneC(O)NH and NHC₁₋₁₀alkyleneC(O)NH. In some embodiments, L¹ is selected from C₁₋₁₀alkyleneC(O)NH, C₁₋₁₀alkyleneO, C₁₋₁₀alkyleneC(O)NH and C₁₋₁₀alkyleneO.

In some embodiments, L² is selected from C₁₋₁₀alkylene and C₁₋₁₀alkyleneS.

In some embodiments, the reactive functional groups of R¹ and R⁴ are nucleophilic and are reactive to a complementary electrophilic group present on the compound to be attached. Useful electrophilic groups on the compound include, but are not limited to, aldehyde, olefin, acetylene, carboxylic acid, ester and ketone functional groups. In some embodiments, the reactive functional groups of R¹ and R⁵ are electrophilic and are reactive to a complementary nucleophilic group present on the compound to be attached. Useful nucleophilic groups on the compound include, but are not limited to, hydrazide, oxime, amino, thiol, hydrazine, thiosemicarbazone, hydrazine carboxylate and aryl hydrazide. In some embodiments, the nucleophilic group is selected from amino and thiol groups provided by reactive lysine and cysteine amino acid groups, respectively.

In some embodiments, the nucleophilic and electrophilic reactive functional groups of R¹ and R⁴ include, but are not limited to, Michael addition acceptors, olefins, acetylenes, alcohols, phenols, ethers, oxides, halides, aldehydes, ketones, carboxylic acids, esters, amines, thiols, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids, isonitriles, amidines, imides, imidates, nitrones, hydroxylamines, oximes, hydroxamic acids, thiohydroxamic acids, allenes, ortho esters, N-hydroxysuccinimide esters, maleimide, sulfites, enamines, ureas, semicarbazides, carbodiimides, carbamates, imines, azides, azo compounds and nitroso compounds.

In some embodiments, the reactive functional groups of R¹ and R⁴ are independently selected from a nucleophilic group and an electrophilic group. In some embodiments, the reactive functional groups of R¹ and R⁴ are selected from Michael addition acceptors, N-hydroxysuccinimide esters, amines, maleimide and thiols.

To attach different entities on each side of the linkers of the application it is desirable that each of the reactive functional groups in R¹ and R⁴ have different reactivities so that one of R¹ and R⁴ can be functionalized by reaction with a complementary functional group in the presence of the other of R¹ and R⁴, and without the other of R¹ and R⁴ participating in the reaction. In some embodiments, one of R¹ and R⁴ is masked or in protected form (i.e. comprising a protecting group) to prevent it from reacting while the other of R¹ and R⁴ is being functionalized and the masking or protecting group is removed after the first reaction and functionalization is complete.

In some embodiments, the compound of Formula (I) has the following structure:

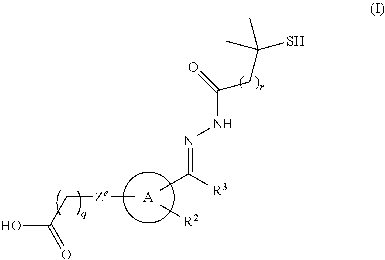

or a pharmaceutically acceptable salt and/or solvate thereof,

wherein

Ring A, R² and R³ are as defined above;

Z^{e} is selected from C(O)NH and O; and

q and r are independently 1, 2, 3, 4, 5, 6, 7 or 8.

In some embodiments, the compound of Formula (I) has the following structure:

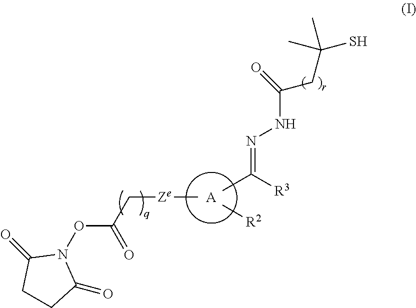

or a pharmaceutically acceptable salt and/or solvate thereof,

wherein

Ring A, R² and R³ are as defined above;

Z^{e} is selected from C(O)NH and O; and

q and r are independently 1, 2, 3, 4, 5, 6, 7 or 8.

In some embodiments, q is 2, 3 or 4. In some embodiments, q is 3. In some embodiments, r is 1 or 2. In some embodiments, r is 1.

In some embodiments Z is O. In some embodiments Z is C(O)NH.

In some embodiments, the compound of Formula (I) is selected from:

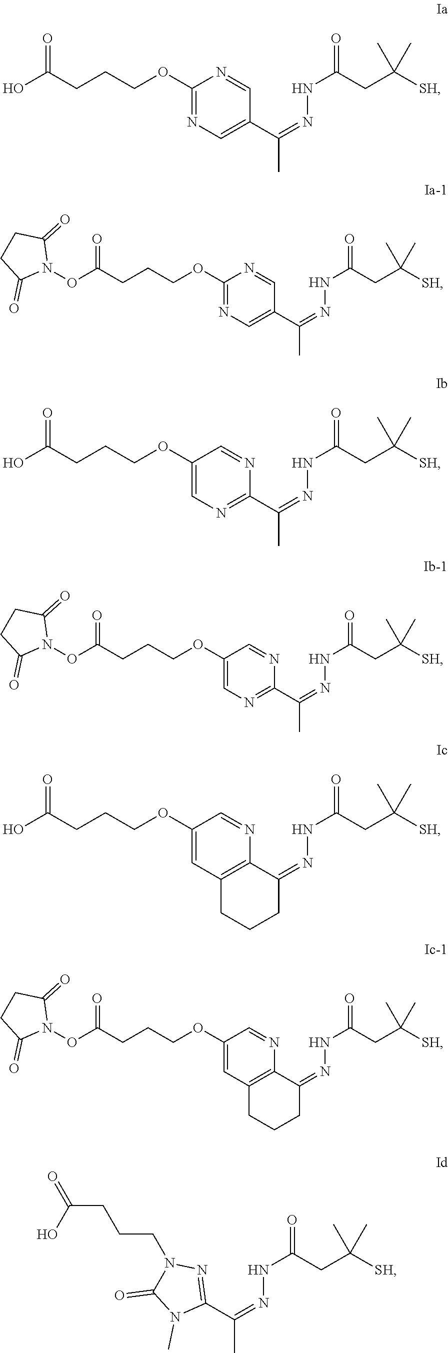

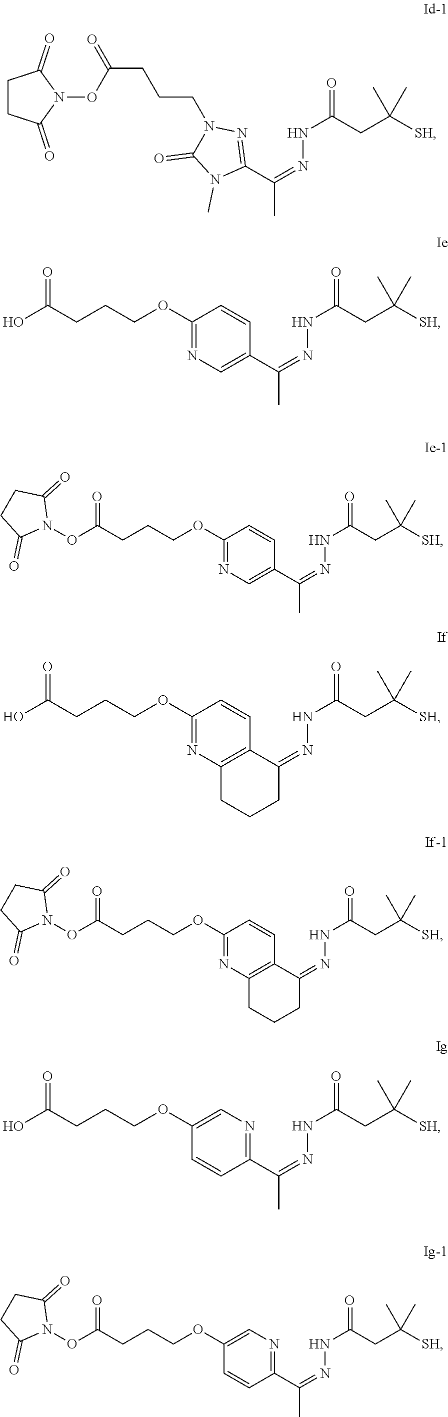

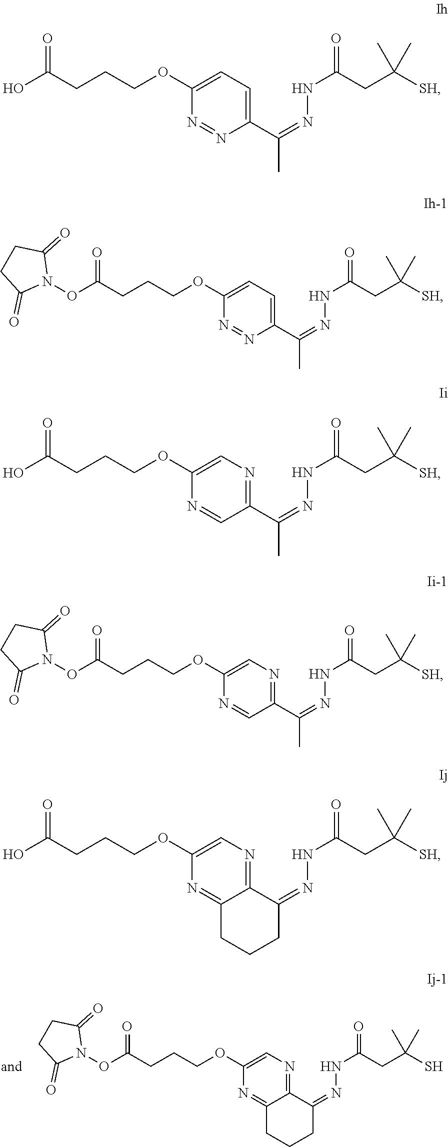 [SMILES: C/C(=N/NC(=O)CC(C)(C)S)c1ccc(OCCCC(=O)O)nn1 ; C/C(=N/NC(=O)CC(C)(C)S)c1ccc(OCCCC(=O)ON2C(=O)CCC2=O)nn1 ; C/C(=N/NC(=O)CC(C)(C)S)c1cnc(OCCCC(=O)O)cn1 ; C/C(=N/NC(=O)CC(C)(C)S)c1cnc(OCCCC(=O)ON2C(=O)CCC2=O)cn1 ; CC(C)(S)CC(=O)N/N=C1/CCCc2nc(OCCCC(=O)O)cnc21 ; CC(C)(S)CC(=O)N/N=C1/CCCc2nc(OCCCC(=O)ON3C(=O)CCC3=O)cnc21]

or a pharmaceutically acceptable salt and/or solvate thereof.

The present application also includes a compound of Formula (II):

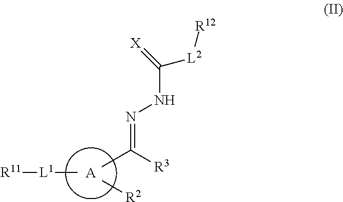

or a pharmaceutically acceptable salt and/or solvate thereof,

wherein:

R¹¹ and R¹² are different and are selected from compounds to be linked together, and

Ring A, L¹, L², R², R³ and X are as defined above for Formula (I).

In some embodiments, R¹¹ and R¹² are independently selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex and solid support. In some embodiments, R¹¹ and R¹² are independently selected from a fluorescent dye, drug, small molecule, antibody, lipid, carbohydrate, nucleic acid, peptide, radiolabel, PET label, nanoparticle, polymer, macrocycle and metal complex.

In some embodiments, R¹¹ and R¹² are independently selected from an antibody and drug. In some embodiments, R¹¹ is an antibody and R¹² is a drug.

In some embodiments, the compound of Formula (II) is for targeting a binding moiety, a labelling agent and/or a therapeutic agent to a specific site in the body of a subject. Accordingly, in some embodiments, R¹¹ and R¹² are complementary or dependent on the identity of each other. For example, if R¹² is a payload such as a drug or a label, then R¹¹ is a complementary group such as a binding moiety targeting a specific site in the body (a ligand specific for a receptor or an antibody specific for an antigen) which can deliver the payload to that specific site in the body.

In some embodiments, one of R¹¹ and R¹² is an antibody and the other of R¹¹ and R¹² is a drug. In some embodiments, R¹¹ is an antibody and R¹² is a drug. In some embodiments, the antibody binds to one or more tumor-associated antigens. In some embodiments, the antibody binds to one or more tumor-associated cell-surface receptors and the drug is a drug for treating cancer.

In some embodiments, the antibody is any antibody of therapeutic value. In some embodiments, the antibody is a wild type antibody amenable to cysteine or lysine conjugation. In some embodiments, the antibody is bio-engineered for site specific conjugation to enable a more controlled DAR ratio.

In some embodiments, the antibody is of the immunoglobulin (Ig) type. The immunoglobulin can be of any type (e.g., IgG, IgE, IgM, IgD and IgA), class (e.g., IgG1, IgG2, IgG3, IgG4, IgA1 and IgA2) or subclass of immunoglobulin molecule.

In some embodiments, the antibody specifically binds to a receptor encoded by an ErbB gene. In some embodiments, the antibody specifically binds to an ErbB receptor selected from EGFR, HER2, HER3 and HER4. In some embodiments, the tumor-associated cell-surface receptor is an ErbB receptor. In some embodiments, the antibody specifically binds to the EGFR receptor.

In some embodiments, the antibody is a monoclonal antibody of the IgG isotype. In some embodiments, the antibody is a chimeric antibody. In some embodiments, the antibody is selected from zalutumumab, nimotuzumab, matuzumab and cetuximab. In some embodiments, the antibody is cetuximab. In some embodiments, the antibody is trastuzumab.

In some embodiments, the drug is a drug for treating cancer. In some embodiments, the drug is selected from a protein kinase inhibitor, proteasome inhibitor, topoisomerase inhibitor, aromatase inhibitor, anthracycline, tubulin inhibitor, a nicotinamide phosphoribosyltransferase (NAMPT) inhibitor, DNA binding molecule and an alkylating agent. In some embodiments, the drug is a tubulin inhibitor. In some embodiments, the drug is monomethyl auristatin E (MMAE). In some embodiments, the drug is a macrolide. In some embodiments, the drug is a maytansinoid. In some embodiments, the drug is DM1. In some embodiments, the drug is a DNA binding agent from the pyrrolobenzodiazepine family.

In some embodiments, the drug is an anticancer drug. In some embodiments, the anticancer drug is a thiol-containing anticancer drug or a calicheamicin derivative. In some embodiments, the thiol containing anticancer drug is a maytansinoid, such as DM1. In some embodiments, the drug is a DNA binding agent selected from the pyrrolobenzodiazepine family. In some embodiments, the anticancer drug is a tubulin polymerization inhibitor. In some embodiments, the drug is MMAE.

In some embodiments, the compound of Formula (II) has the following structure:

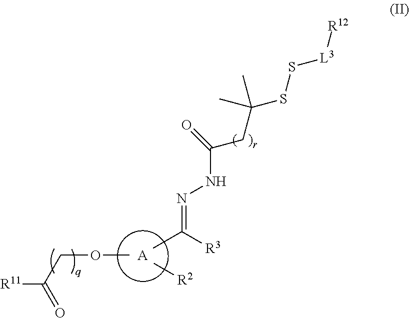

or a pharmaceutically acceptable salt and/or solvate thereof,

wherein

Ring A, R² and R³ are as defined above for Formula (I);

R¹¹ and R¹² are independently selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex and solid support;

L³ is a linker moiety;

q is 1, 2, 3, 4, 5, 6, 7 or 8; and

r is 1, 2, 3, 4, 5, 6, 7 or 8.

In some embodiments, the compound of Formula (II) has the following structure:

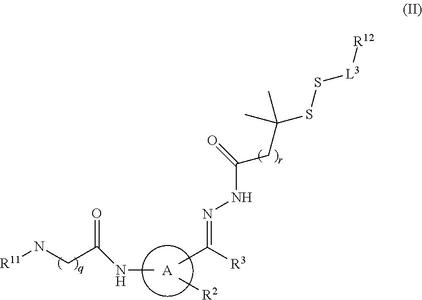

or a pharmaceutically acceptable salt and/or solvate thereof,

wherein

Ring A, R² and R³ are as defined above for Formula (I);

R¹¹ and R¹² are independently selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex and solid support;

L³ is a linker moiety;

q is 1, 2, 3, 4, 5, 6, 7 or 8; and

r is 1, 2, 3, 4, 5, 6, 7 or 8.

In some embodiments, q is 2, 3 or 4. In some embodiments q is 3. In some embodiments, r is 1 or 2. In some embodiments, r is 1.

In some embodiments L³ is selected from a direct bond, Z^{b}, R^{e}, Z^{b}—R^{c}, R^{c}—Z^{b}, R^{c}—Z^{b}—R^{d} and Z^{b}—R^{c}—Z°, wherein Z^{b} and Z° are independently selected from O, S, S(O), SO₂, NH, N(C₁₋₆alkyl), C(Q^{a}), C(Q^{a})Y^{b}, Y^{b}C(Q^{a}), Y^{b}C(Q^{a})Y^{c}, (C₁₋₆alkyleneY^{b})ₚ and Y^{b}—(C₁₋₆alkyleneY^{b})ₚ, wherein R^{c} and R^{d} are independently selected from C₁₋₁₀alkylene, C₂₋₁₀alkenylene and C₂₋₁₀alkynylene; Q^{a}, Y^{b} and Y^{c} are independently selected from O, S, NH and N(C₁₋₆alkyl); and p is selected from 1, 2, 3, 4, 5 and 6.

In some embodiments, R^{c} and R^{d} are independently selected from C₁₋₆alkylene, C₂₋₆alkenylene and C₂₋₆alkynylene. In some embodiments, R^{c} and R^{d} are independently selected from C₁₋₆alkylene.

In some embodiments, Q^{a}, Y^{b} and Y° are independently selected from O, S, NH and N(CH₃).

In some embodiments Z^{b} and Z° are independently selected from O, S, S(O), SO₂, NH, N(CH₃), C(O), C(O)NH, NHC(O), NHC(O)O, OC(O)O, NHC(O)NH, OC(O)NH, NHC(NH)NH, (C₁₋₆alkyleneO)ₚ and O—(C₁₋₆alkyleneO)ₚ.

In some embodiments L³ is selected from OC(O)C₁₋₁₀alkyleneO, NHC(O)C₁₋₁₀alkyleneO, C₁₋₆alkyleneO, OC(O)C₁₋₁₀alkyleneNH, NHC(O)C₁₋₁₀alkyleneNH, C₁₋₆alkyleneNH, C(O)C₁₋₁₀alkyleneO and C(O)C₁₋₁₀alkyleneNH.

In some embodiments, the half-life of the compounds of Formula (II) is controlled by the substituent selection for R² and/or R³.

In a further aspect, the present application also includes a compound of Formula (III):

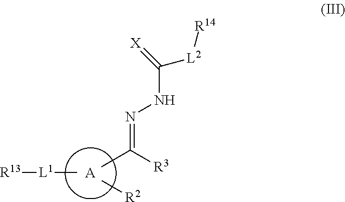

or a pharmaceutically acceptable salt and/or solvate thereof, wherein one of R¹³ and R¹⁴ is a reactive functional group; and the other of R¹³ and R¹⁴ is a compound to be linked to another same or different compound; and Ring A, R², R³, X, L¹ and L² are as defined above for Formula (I) and (II).

In some embodiments, the compound of Formula (III) has the following structure:

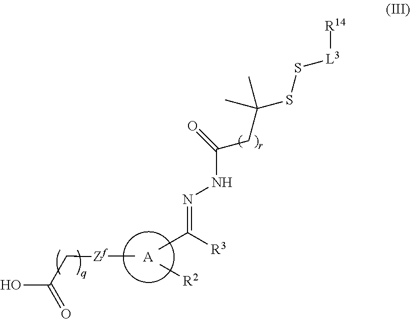

wherein R¹⁴ is a compound to be linked to another same or different compound; Z^{f} is C(O)NH or O; and

Ring A, R², R³, L³, q and r are as defined above for Formula (I) and (II), or a pharmaceutically acceptable salt and/or solvate thereof.

In some embodiments, the compound of Formula (III) is a compound of the following structure;

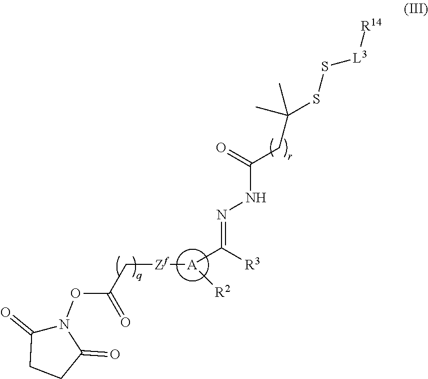

wherein R¹⁴ is a compound to be linked to another same or different compound as defined in Formula (II);

Ring A, R², R³, L³, q and r are as defined above for Formula (I) and (II); and

Z^{f} is C(O)NH or O;

or a pharmaceutically acceptable salt and/or solvate thereof.

In some embodiments, the compound of Formula (III) is selected from:

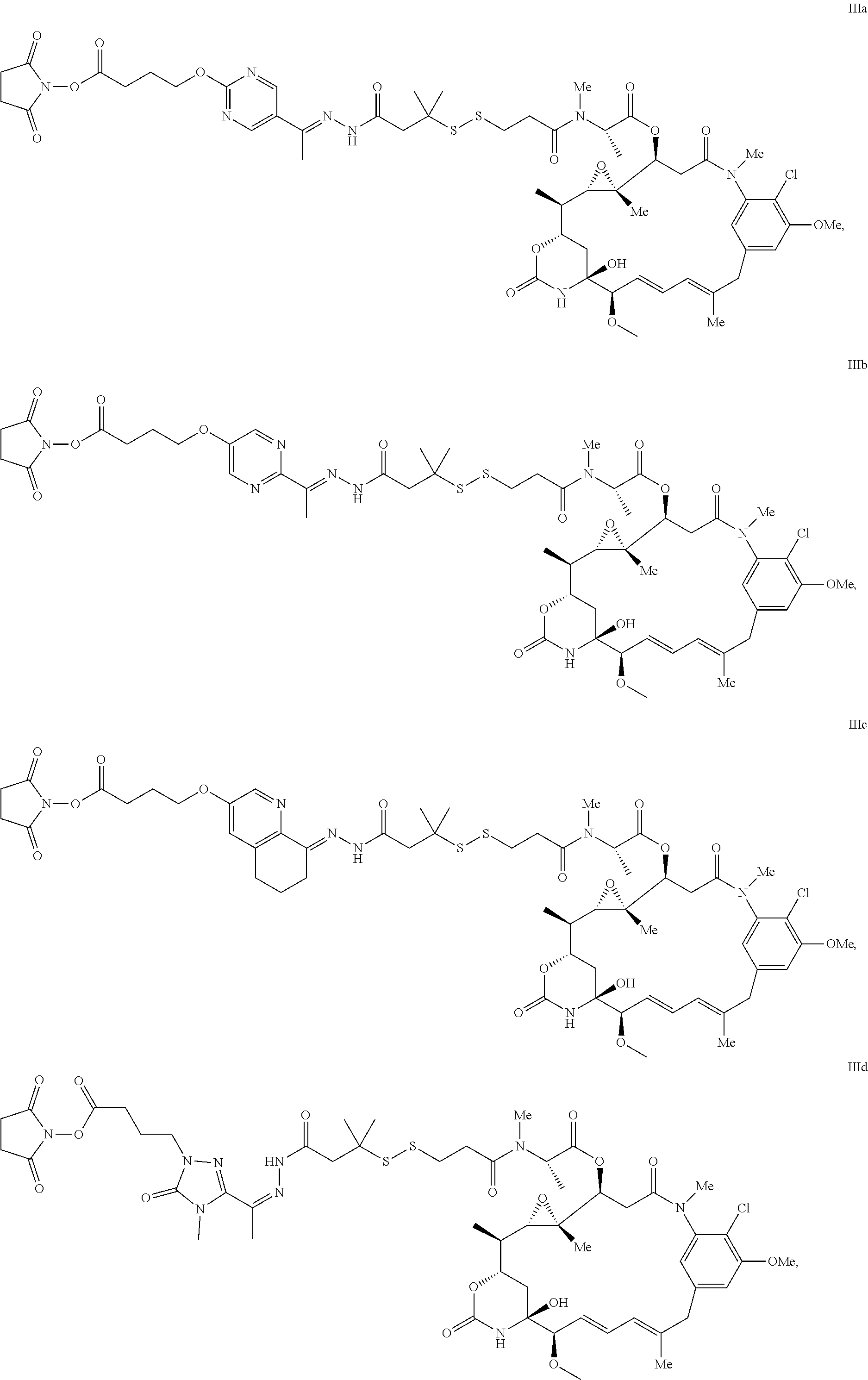

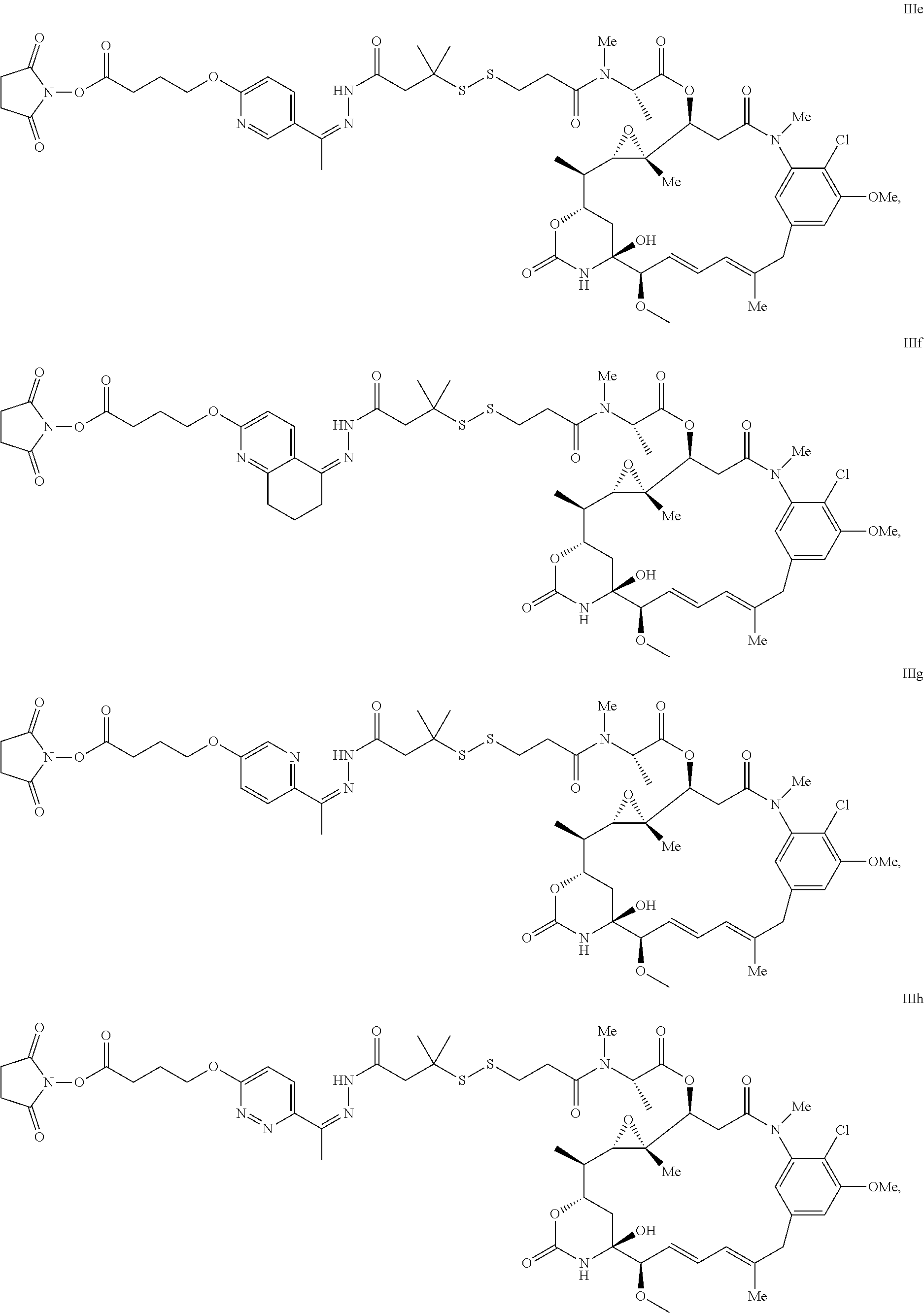

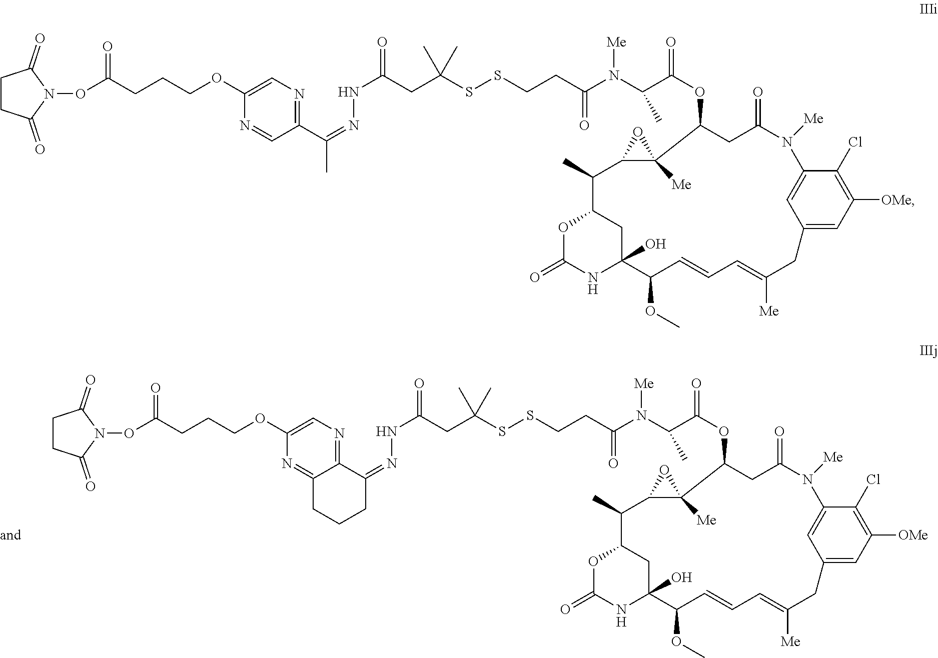 [SMILES: COc1cc2cc(c1Cl)N(C)C(=O)C[C@H](OC(=O)[C@H](C)N(C)C(=O)CCSSC(C)(C)CC(=O)N/N=C(/C)c1cnc(OCCCC(=O)ON3C(=O)CCC3=O)cn1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)CCSSC(C)(C)CC(=O)N/N=C1/CCCc3nc(OCCCC(=O)ON4C(=O)CCC4=O)cnc31)[C@]1(C)O[C@H]1[C@H](C)[C@@H]1C[C@@](O)(NC(=O)O1)[C@H](OC)/C=C/C=C(\C)C2]

or a pharmaceutically acceptable salt and/or solvate thereof.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may exist as mixtures of E and Z isomers or cis and trans isomers and it is intended that any above mentioned isomer, as well as mixtures thereof, are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of the application include, for example, those made with solvents that are pharmaceutically acceptable. Examples of such solvents include water (resulting solvate is called a hydrate) and ethanol and the like.

### III. Antibody-Drug Conjugates of the Application

The present application includes an antibody-drug conjugate (ADC) comprising an antibody covalently attached by a linker to one or more drugs, the conjugate having a Formula (IV):

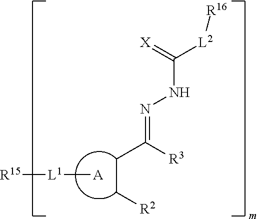

or a pharmaceutically acceptable salt and/or solvate thereof,

wherein

R¹⁵ is an antibody;

R¹⁶ is a drug;

Ring A, L¹, L², X, R² and R³ are as defined as above for Formula (I); and

m is an integer from 1 to 20.

In some embodiments, the compound of Formula (IV) has the following structure:

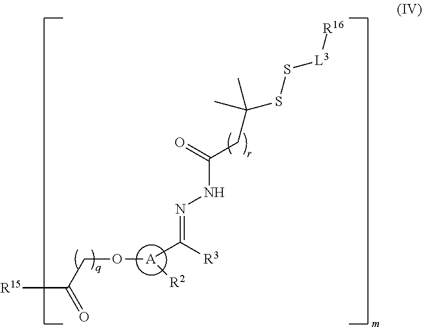

wherein

R¹⁵ is an antibody;

R¹⁶ is a drug;

Ring A, L³, R² and R³ are as defined as above for Formula (I);

L³ is a linker moiety;

q is 1, 2, 3, 4, 5, 6, 7 or8;

r is 1, 2, 3, 4, 5, 6, 7 or 8; and

m is an integer from 1 to 20,

or a pharmaceutically acceptable salt and/or solvate thereof.

In some embodiments, q in the compounds of Formula (IV) is 2, 3 or 4. In some embodiments, q in the compounds of Formula (IV) is 3. In some embodiments, r in the compounds of Formula (IV) is 1 or 2. In some embodiments, r in the compounds of Formula (IV) is 1. In some embodiments, R³ is CH₃.

In some embodiments in the compounds of Formula (IV) L³ is selected from a direct bond, Z^{b}R^{c}, Z^{b}—R^{c}, R^{c}—Z^{b}, R^{c}—Z^{b}—R^{d} and Z^{b}—R^{c}—Z°, wherein Z^{b} and Z° are independently selected from O, S, S(O), SO₂, NH, N(C₁₋₆alkyl), C(Q^{a}), C(Q^{a})Y^{b}, Y^{b}C(Q^{a}) Y^{b}C(Q^{a})Y^{c}, (C₁₋₆alkyleneY^{b})ₚ and Y^{b}—(C₁₋₆alkyleneY^{b})ₚ, wherein R^{c} and R^{d} are independently selected from C₁₋₁₀alkylene, C₂₋₁₀alkenylene and C₂₋₁₀alkynylene; Q^{a}, Y^{b} and Y^{c} are independently selected from O, S, NH and N(C₁₋₆alkyl); and p is selected from 1, 2, 3, 4, 5 and 6.

In some embodiments in the compounds of Formula (IV), R^{c} and R^{d} are independently selected from C₁₋₆alkylene, C₂₋₆alkenylene and C₂₋₆alkynylene. In some embodiments, R^{c} and R^{d} are independently selected from C₁₋₆alkylene.

In some embodiment in the compounds of Formula (IV), Q^{a}, Y^{b} and Y° are independently selected from O, S, NH and N(CH₃).

In some embodiments in the compounds of Formula (IV), Z^{b} and Z^{c} are independently selected from O, S, S(O), SO₂, NH, N(CH₃), C(O), C(O)NH, NHC(O), NHC(O)O, OC(O)O, NHC(O)NH, OC(O)NH, NHC(NH)NH, (C₁₋₆alkyleneO)ₚ and O—(C₁₋₆alkyleneO)ₚ,

In some embodiments, the antibody binds to one or more tumor-associated antigens. In some embodiments, the antibody binds to one or more tumor-associated cell-surface receptors. In some embodiments, the antibody specifically binds to a receptor encoded by an ErbB gene. In some embodiments, the tumor-associated cell-surface receptor is an ErbB receptor.

In some embodiments, the antibody specifically binds to an ErbB receptor selected from EGFR, HER2, HER3 and HER4. In some embodiments, the antibody specifically binds to the EGFR receptor. In some embodiments, the antibody is a monoclonal antibody. In some embodiments, the antibody is a chimeric antibody. In some embodiments, the antibody is selected from zalutumumab, nimotuzumab, matuzumab and cetuximab. In some embodiments, the antibody is cetuximab. In some embodiments, the antibody is trastuzumab.

In some embodiments, the drug is a drug for targeting cancer. In some embodiments, the drug is selected from a protein kinase inhibitor, proteasome inhibitor, topoisomerase inhibitor, aromatase inhibitor, anthracycline, tubulin inhibitor, a nicotinamide phosphoribosyltransferase (NAMPT) inhibitor, DNA binding molecule and an alkylating agent. In some embodiments, the drug is a tubulin inhibitor. In some embodiments, the drug is a macrolide. In some embodiments, the drug is a maytansinoid. In some embodiments, the one or more drug moieties is DM1. In some embodiments, the drug is a DNA binding agent from the pyrrolobenzodiazepine family.

In some embodiments, the drug is an anticancer drug. In some embodiments, the anticancer drug is a thiol-containing anticancer drug or a calicheamicin derivative. In some embodiments, the thiol containing anticancer drug is a maytansinoid, such as DM1. In some embodiments, the drug is a DNA binding agent from the pyrrolobenzodiazepine family. In some embodiments, the anticancer drug is a tubulin polymerization inhibitor. In some embodiments, the drug is MMAE.

The drug loading of ADCs is represented by the integer m, which indicates the average number of drugs conjugated per antibody in the conjugate of Formula (III). The drug to antibody (DAR) ratio is relevant for the preparation of ADC's, as higher drug loading, e.g. m>5, may cause aggregation, insolubility, toxicity or loss of cellular permeability. Further, the DAR ratio is dependent upon the number of reactive sites present on the antibody. For example, where the attachment point is a cysteine thiol or lysine amine, as in the exemplary embodiments of the present application, an antibody may have only one or few number of these reactive groups through which a linker maybe attached. Additionally, the antibody may be subjected to denaturing conditions to reveal reactive nucleophilic groups such as lysine and cysteine. In some embodiments, the DAR ratio of the compounds of Formula (IIb) ranges from 1 to 20 drugs per antibody.

In some embodiments, m is an integer from 1 to 10. In some embodiments, m is an integer from 1 to 5.

Known antibodies for the treatment and prevention of cancer can be conjugated as ADCs. Antibodies immunospecific for a cancer cell antigen are obtained commercially or produced by any method known to a person skilled in the art, including, e.g., chemical syntheses or by recombinant expression techniques. In some embodiments, the nucleotide sequence encoding antibodies immunospecific for a cancer cell antigens is obtained, for example, from the GenBank database or a similar nucleotide sequence database, literature publications, or through routine cloning and sequencing.

In some embodiments, the ADCs of the present application selectively deliver an effective dose of a cytotoxic agent, such as a drug, to tumor tissue with greater selectivity, i.e., a lower effective dose is achieved, than upon delivery of the same dose of drug not conjugated to an antibody.

In some embodiments, the drug of the compound of Formula (IV) is not cleaved from the antibody until the compound enters a cell with a cell-surface receptor specific for the antibody of the compound, at which time the drug is cleaved from the antibody. In some embodiments, the drug is intracellularly cleaved from the antibody of the compound of Formula (IV) through enzymatic action, hydrolysis, oxidation or pH conditions. In some embodiments, the acidic half-life in lysosomal environments of the compounds of Formula (IV) is controlled by the substituents on Ring A.

In some embodiments, the compound of Formula (IV) is selected from:

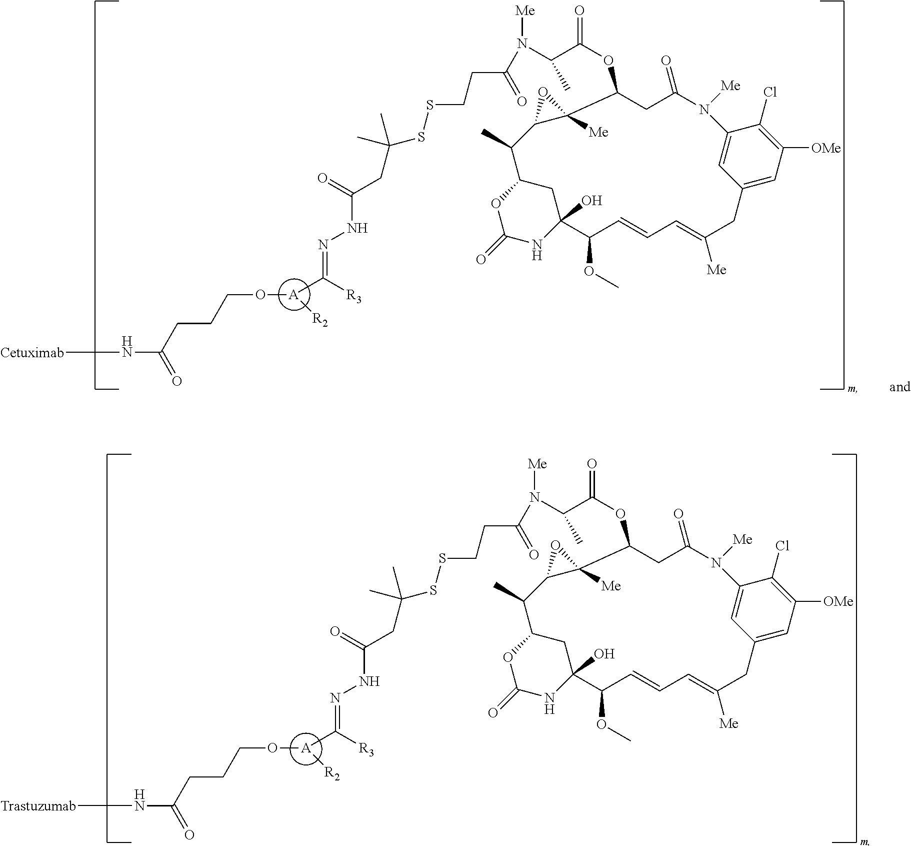

wherein R² and R³ are as defined above, and m=1 to 20, or a pharmaceutically acceptable salt and/or solvate thereof.

In some embodiments, the compound of Formula (IV) is selected from:

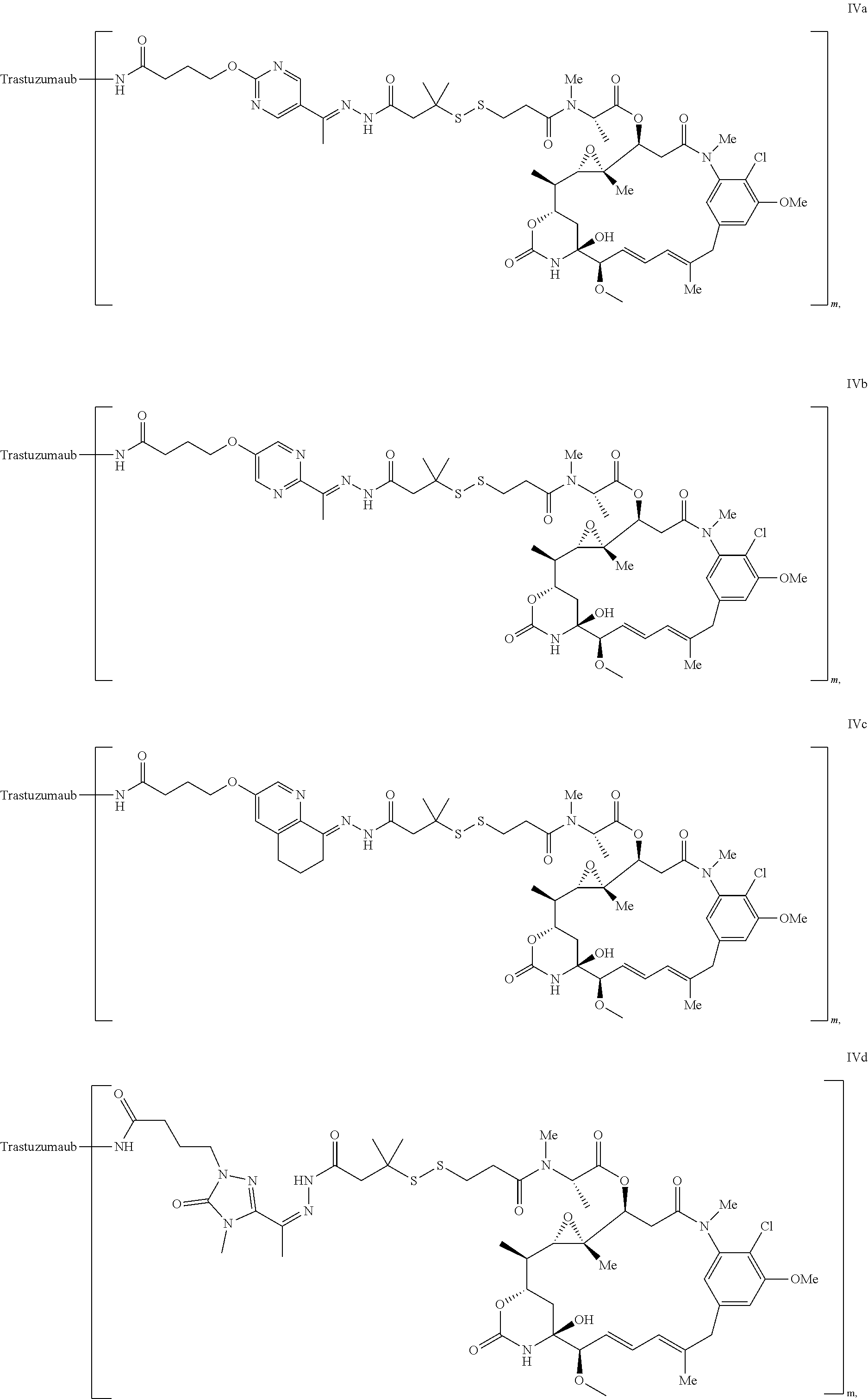

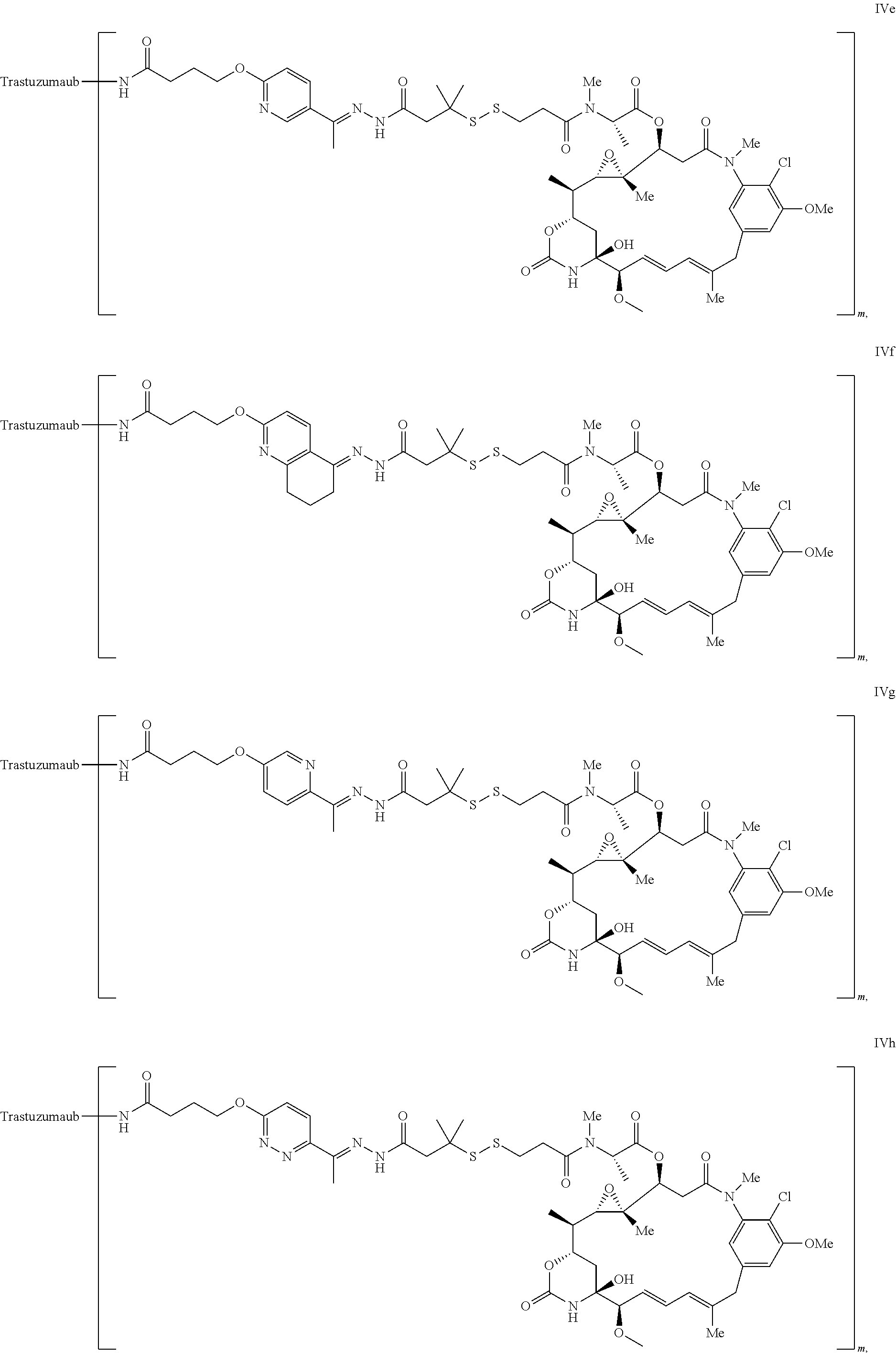

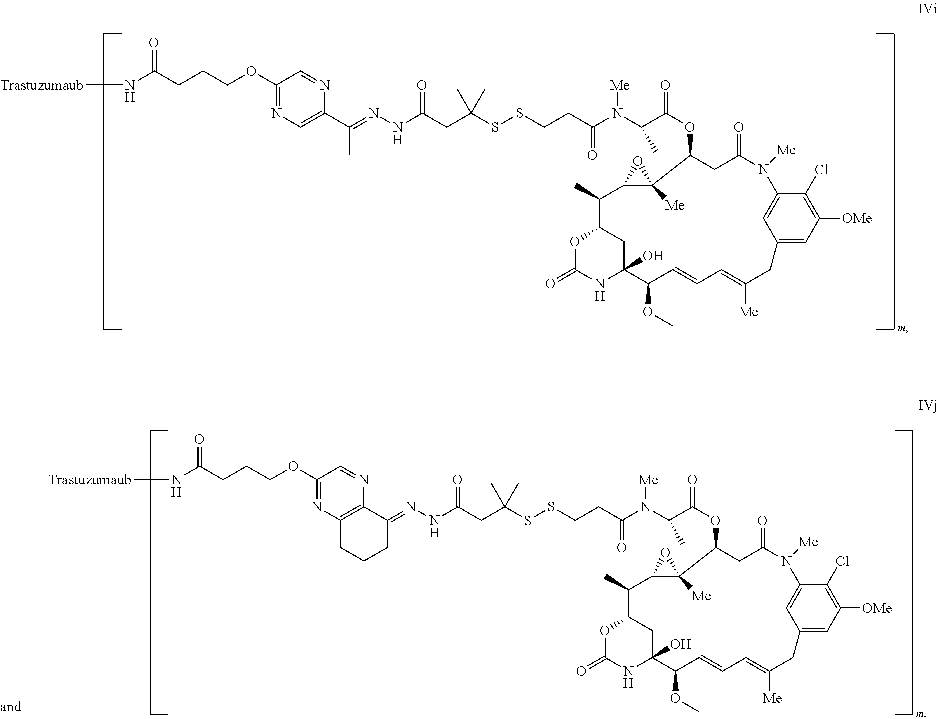

wherein m is from 1 to 10, or from 2 to 5,

or a pharmaceutically acceptable salt and/or solvate thereof.

In some embodiments, the compound of Formula (IV) is selected from:

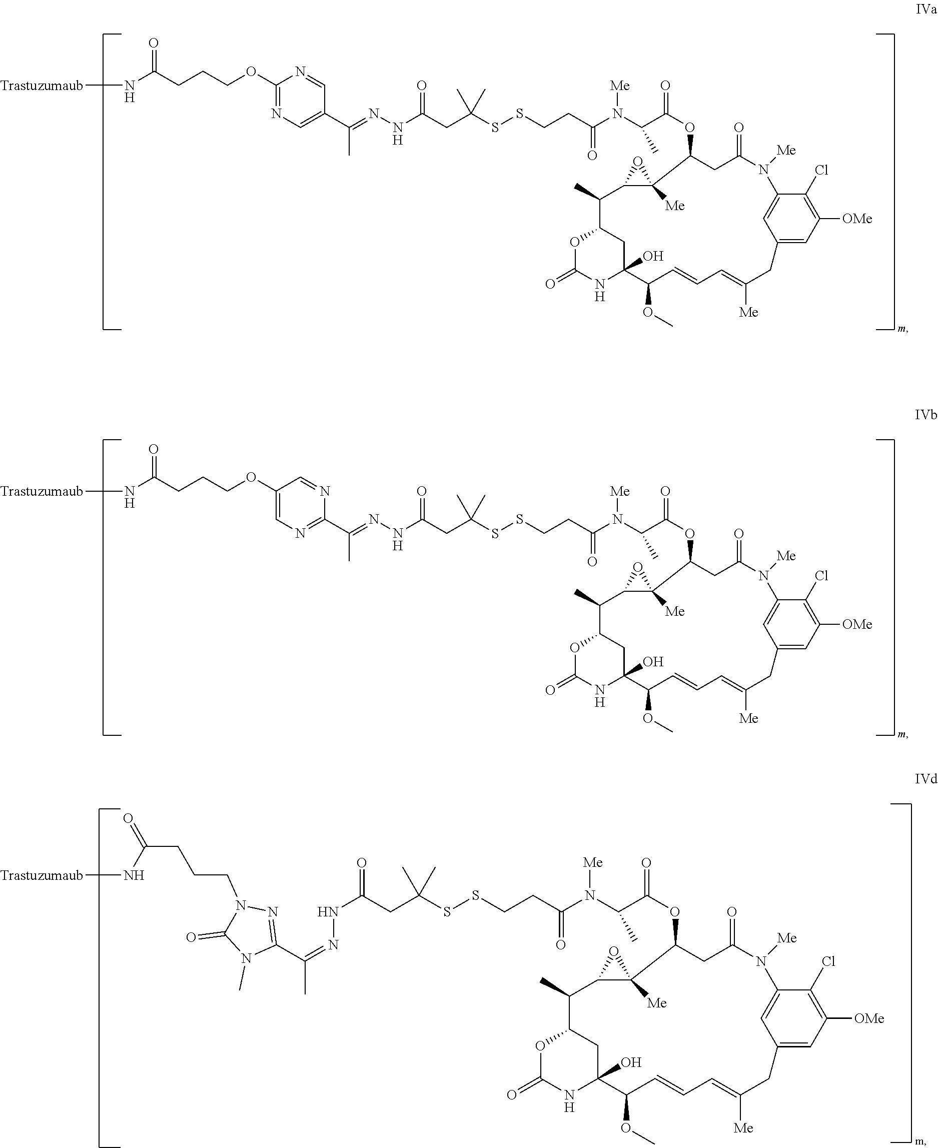

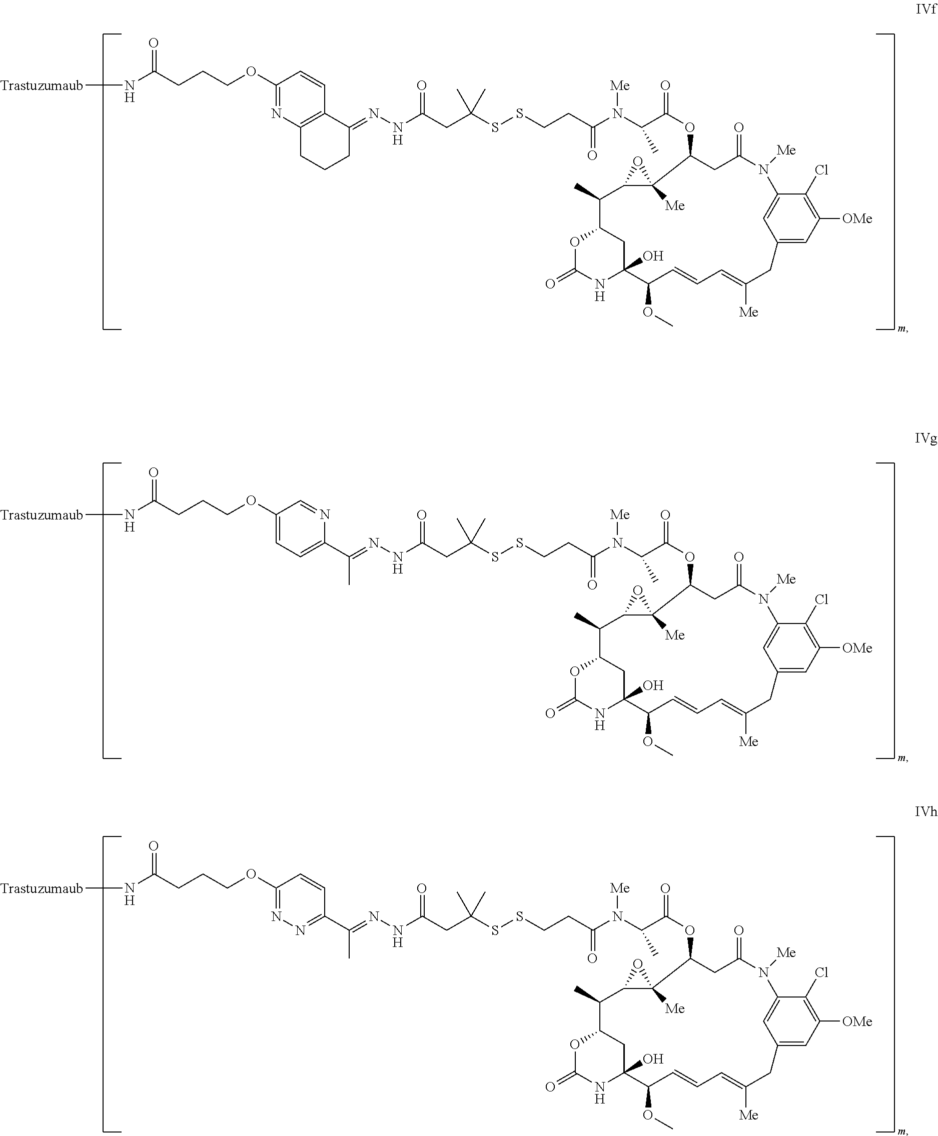

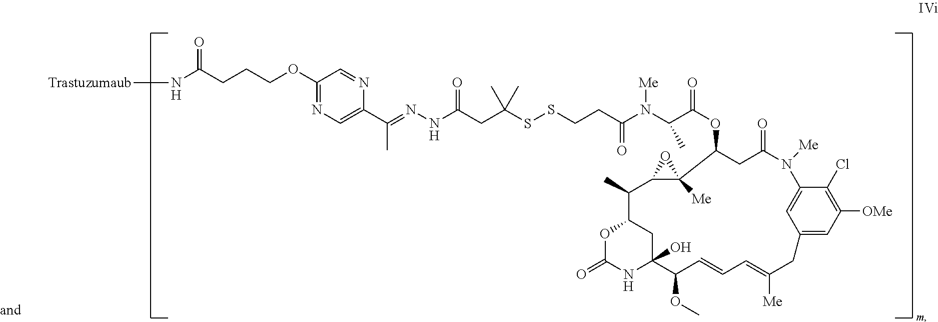

wherein m is from 1 to 10, or from 2 to 5,

or a pharmaceutically acceptable salt and/or solvate thereof.

### IV. Methods of Preparing Compounds of the Application

Scheme (A) illustrates one embodiment of a route to compounds of the application in which a functionalized hydrazide is formed from commercially available compounds A, wherein R⁴ is a reactive functional group or a protected form thereof and X and L² are as defined in Formula (I) to afford intermediates B. The subsequent coupling of B with aromatic compounds C, wherein Ring A, R¹, R², R³ and L¹ are as defined in Formula (I) and in which R¹ may be in protected form, provides compounds of the application.

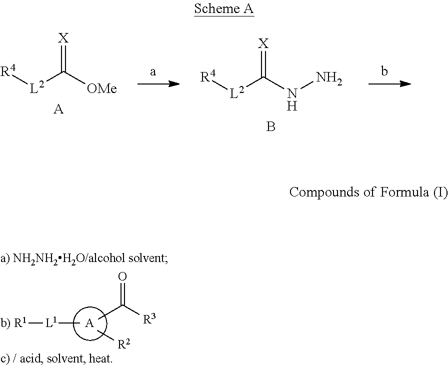

Compounds of Formula C are either commercially available or are synthesized from commercially available compounds using methods known in the art, for example starting from compounds of Formula D:

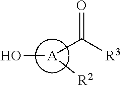

wherein Ring A, R² and R³ are as defined in Formula (I).

In some embodiments, the reactive functional groups R¹ and R⁴ of the compounds of Formula (I) are subsequently conjugated to a complementary reactive functional group of compounds to be linked, for example, a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex or solid support, to produce the compounds of Formula (II), (III) or (IV) of the present application.

Accordingly, in another aspect, the present application includes a method of synthesizing one or more compounds of Formula (II), (III) or (IV) as defined above, or a pharmaceutically acceptable salt and/or solvate thereof, wherein the method comprises reacting one or more compounds of Formula (I) as defined above with a first compound to be linked, for example, selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex or solid support, and then a second, different compound to be linked, for example, selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex and solid support.

To attach different entities on each side of the hydrazine linkers of the application it is desirable that each of the reactive functional groups in R¹ and R⁴ have different reactivities so that one of R¹ and R⁴ can be functionalized by reaction with a complementary functional group in the presence of the other of R¹ and R⁴, and without the other of R¹ and R⁴ participating in the reaction. In some embodiments, one of R¹ and R⁴ is masked or in protected form (i.e. comprises a protecting group) to prevent it from reacting while the other of R¹ and R⁴ is being functionalized and the masking or protecting group is removed after the first reaction and functionalization is complete.

For preparing ADC compounds of Formula (IV) of the application, in some embodiments, a compound of Formula (I)-drug conjugate is first prepared. Methods for conjugating a Formula (I)-drug conjugate to an antibody and purifying the ADCs are known to those skilled in the art.

Accordingly, in another aspect the present application includes a method of preparing an ADC of Formula (IV) comprising:

(a) reacting a compound of Formula (I) with a drug to provide a Formula (I)-drug conjugate;

(b) reacting the Formula (I)-drug conjugate with an antibody to provide the ADC of Formula (IV); and optionally

(c) purifying the ADC of Formula (IV).

In another aspect, the present application includes a method of preparing an ADC of Formula (IV) comprising:

(a) reacting a compound of Formula (III) as defined above with an antibody to provide the ADC of Formula (IV) and optionally

(b) purifying the ADC of Formula (IV).

The present application also includes a use of a compound of Formula (I) or (III) to prepare an ADC.

In some embodiments, the resulting ADC products are isolated or purified using known methods, such as for example, lyophilization, chromatography, precipitation, filtration, microfluidic and/or liquid chromatography separation methods.

In some embodiments, the drug is an anticancer drug. In some embodiments, the anticancer drug is a thiol-containing anticancer drug or a calicheamicin derivative. In some embodiments, the thiol containing anticancer drug is a maytansinoid, such as DM1. In some embodiments, the drug is a DNA binding agent from the pyrrolobenzodiazepine family.

### V. Compositions of the Application

The compounds of the application are suitably formulated in a conventional manner into compositions using one or more carriers. Accordingly, the present application also includes a composition comprising one or more compounds of the application and a carrier. The compounds of Formula (II) and/or (IV), or pharmaceutically acceptable salts and/or solvates thereof, are suitably formulated into pharmaceutical compositions for administration to subjects in a biologically compatible form suitable for administration in vivo. Accordingly, the present application further includes a pharmaceutical composition comprising one or more compounds of Formula (II) and/or (IV), or pharmaceutically acceptable salts and/or solvates thereof, and a pharmaceutically acceptable carrier. In embodiments of the application the pharmaceutical compositions are used in the treatment and/or diagnosis of any of the diseases, disorders or conditions described herein.

The compounds of Formula (II) and/or (IV), or pharmaceutically acceptable salts and/or solvates thereof, are administered to a subject in a variety of forms depending on the selected route of administration, as will be understood by those skilled in the art. For example, compounds of Formula (II) and/or (IV), or pharmaceutically acceptable salts and/or solvates thereof, are administered by oral, inhalation, parenteral, buccal, sublingual, nasal, rectal, vaginal, patch, pump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, compounds of Formula (II) and/or (IV), or pharmaceutically acceptable salts and/or solvates thereof, are orally administered, for example, with an inert diluent or with an assimilable edible carrier, or are enclosed in hard or soft shell gelatin capsules, or are compressed into tablets, or are incorporated directly with the food of the diet. In some embodiments, the compounds are incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compounds are protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compounds of (II) and/or (IV), or pharmaceutically acceptable salts and/or solvates thereof, are suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of Formula (II) and/or (IV), or pharmaceutically acceptable salts and/or solvates thereof, and use the lyophilizates obtained, for example, for the preparation of products for injection.

In some embodiments, the compounds of Formula (II) and/or (IV), or pharmaceutically acceptable salts and/or solvates thereof, are administered parenterally. For example, solutions of compounds of Formula (II) and/or (IV), or pharmaceutically acceptable salts and/or solvates thereof, are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of Formula (II) and/or (IV), or pharmaceutically acceptable salts and/or solvates thereof, are usually prepared, and the pHs of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, compounds of (II) and/or (IV), or pharmaceutically acceptable salts and/or solvates thereof, are formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of Formula (II) and/or (IV), or pharmaceutically acceptable salts and/or solvates thereof, are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of compounds of Formula (II) and/or (IV), or pharmaceutically acceptable salts and/or solvates thereof, and a suitable powder base such as lactose or starch. The aerosol dosage forms can also take the form of a pump-atomizer.

Compositions suitable for buccal or sublingual administration include tablets, lozenges, and pastilles, wherein compounds of Formula (II) and/or (IV), or pharmaceutically acceptable salts and/or solvates thereof, are formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of Formula (II) and/or (IV), or pharmaceutically acceptable salts and/or solvates thereof, are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, Pa., 1980, pp. 1530-1533 for further discussion of suppository dosage forms.

In some embodiments compounds of Formula (II), or pharmaceutically acceptable salts and/or solvates thereof, are coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, compounds of Formula (II), or pharmaceutically acceptable salts and/or solvates thereof, are coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The compounds of Formula (II) and/or (IV), or pharmaceutically acceptable salts and/or solvates thereof, are suitably used on their own but will generally be administered in the form of a pharmaceutical composition in which the one or more compounds of Formula (II) and/or (IV), or pharmaceutically acceptable salts and/or solvates thereof, (the active ingredient) are in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

### VI. Methods and Uses of the Application

Compounds of Formula (II) and/or (IV), or pharmaceutically acceptable salts and/or solvates thereof, comprise a wide variety of active compounds which have possibilities of treating and/or diagnosing a variety of diseases, disorders or conditions.

Accordingly, the present application includes a method of treating and/or diagnosing one or more diseases, disorders or conditions by administering an effective amount of one or more compounds of Formula (II) and/or (IV), or pharmaceutically acceptable salts and/or solvates thereof, to a subject in need thereof. In some embodiments, the disease, disorder or condition depends on the identity of the compounds being conjugated as would be understood by a person skilled in the art.

In some embodiments, the disease, disorder or condition is a neoplastic disorder. Accordingly, the present application also includes a method of treating and/or diagnosing a neoplastic disorder comprising administering a therapeutically effective amount of one or more compounds of Formula (II) and/or (IV), or pharmaceutically acceptable salts and/or solvates thereof, to a subject in need thereof. The present application also includes a use of one or more compounds of Formula (II) and/or (IV), or pharmaceutically acceptable salts and/or solvates thereof, for treatment of and/or diagnosing a neoplastic disorder as well as a use of one or more compounds of Formula (II) and/or (IV), or pharmaceutically acceptable salts and/or solvates thereof, for the preparation of a medicament for treatment of and/or diagnosing a neoplastic disorder. The application further includes one or more compounds of Formula (II) and/or (IV), or pharmaceutically acceptable salts and/or solvates thereof, for use in treating and/or diagnosing a neoplastic disorder. In an embodiment, the treatment is in an amount effective to ameliorate at least one symptom of the neoplastic disorder, for example, reduced cell proliferation or reduced tumor mass, among others, in a subject in need of such treatment.

Neoplasms can be benign (such as uterine fibroids and melanocytic nevi), potentially malignant (such as carcinoma in situ) or malignant (i.e. cancer). Exemplary neoplastic disorders include the so-called solid tumours and liquid tumours, including but not limited to carcinoma, sarcoma, metastatic disorders (e.g., tumors arising from the prostate), hematopoietic neoplastic disorders, (e.g., leukemias, lymphomas, myeloma and other malignant plasma cell disorders), metastatic tumors and other cancers.

In some embodiments, the present application includes a method of treating and/or diagnosing one or more diseases, disorders or conditions mediated by ErbB comprising administering a therapeutically effective amount of one or more compounds of Formula (II) and/or (IV), or pharmaceutically acceptable salts and/or solvates thereof, to a subject in need thereof. The present application also includes a use of one or more compounds of Formula (II) and/or (IV), or pharmaceutically acceptable salts and/or solvates thereof, for treatment of and/or diagnosing one or more diseases, disorders or conditions mediated by ErbB as well as a use of one or more compounds of Formula (II) and/or (IV), or pharmaceutically acceptable salts and/or solvates thereof, for the preparation of a medicament for treatment of and/or diagnosing one or more diseases, disorders or conditions mediated by ErbB.

In some embodiments, the disease, disorder or condition is cancer. Accordingly, the present application also includes a method of treating and/or diagnosing cancer comprising administering a therapeutically effective amount of one or more compounds of Formula (II) and/or (IV), or pharmaceutically acceptable salts and/or solvates thereof, to a subject in need thereof. The present application also includes a use of one or more compounds of Formula (II) and/or (IV), or pharmaceutically acceptable salts and/or solvates thereof, for treatment of and/or diagnosing cancer as well as a use of one or more compounds of Formula (II) and/or (IV), or pharmaceutically acceptable salts and/or solvates thereof, for the preparation of a medicament for treatment of and/or diagnosing cancer. The application further includes one or more compounds of Formula (II) and/or (IV), or pharmaceutically acceptable salts and/or solvates thereof, for use in treating cancer. In an embodiment, the compound is administered for the prevention of cancer in a subject such as a mammal having a predisposition for cancer. In some embodiments, the cancer is an ErbB-expressing cancer. In some embodiments, the subject is human.

In some embodiments, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

In some embodiments, the cancer is selected from ErbB-expressing cancer. In some embodiments, the cancer is selected from breast cancer, skin cancer, prostate cancer, head and neck cancer, colorectal cancer, pancreatic cancer, kidney cancer, lung cancer and brain cancer. In some embodiments of the present application, the cancer is selected from breast cancer, prostate cancer, head and neck cancer, colorectal cancer, pancreatic cancer, kidney cancer, lung cancer and brain cancer.

In a further embodiment, the one or more compounds of the application are administered in combination with one or more additional cancer treatments. In another embodiment, the additional cancer treatment is selected from radiotherapy, chemotherapy, targeted therapies such as antibody therapies and small molecule therapies such as tyrosine-kinase inhibitors, immunotherapy, hormonal therapy and anti-angiogenic therapies.

In some embodiments, when the methods and uses are related to diagnostics, one compound to be linked comprises a binding moiety and the other compound to be linked comprises a labelling agent.

In an embodiment, effective amounts vary according to factors such as the disease state, age, sex and/or weight of the subject. In a further embodiment, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

In an embodiment, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

In an embodiment, the subject is a mammal. In another embodiment, the subject is human.

The compounds of Formula (II) and/or (IV), or pharmaceutically acceptable salts and/or solvates thereof, are either used alone or in combination with other known agents useful for treatment and/or imaging. When used in combination with other agents useful in treatment and/or imaging, it is an embodiment that compounds of Formula (II) and/or (IV), or pharmaceutically acceptable salts and/or solvates thereof, are administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In an embodiment, compounds of Formula (II) and/or (IV), or pharmaceutically acceptable salts and/or solvates thereof, are administered with another therapeutic agent simultaneously or sequentially in separate unit dosage forms or together in a single unit dosage form. Accordingly, the present application provides a single unit dosage form comprising one or more compounds of Formula (II) and/or (IV), or pharmaceutically acceptable salts and/or solvates thereof, an additional therapeutic agent, and a pharmaceutically acceptable carrier.

In some embodiments, the additional therapeutic agent is a chemotherapeutic agent. In some embodiments, the chemotherapeutic agent is selected from the classes of alkylating agents, anthracyclines, cytoskeletal disruptors, epothilones, histone deacetylase inhibitors, topoisomerase inhibitors, kinase inhibitors, nucleotide analogs, peptide antibiotics, platinum-based agents, retinoids, Vinca alkaloids, epigenetic modifiers and immuno-modulators.

The dosage of a compound of the application varies depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. In some embodiments, a compound of the application is administered initially in a suitable dosage that is adjusted as required, depending on the clinical response. Dosages will generally be selected to maintain a serum level of the compound of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg.

### Examples

The following non-limiting examples are illustrative of the present application:

### A. General Methods

Exemplary compounds of the application were synthesized using the methods described herein, or other methods, which are known in the art. Unless otherwise noted, reagents and solvents were obtained from commercial suppliers (e.g. Aldrich, Enamine, Combi-Blocks, Bepharm, J&W PharmLab).

The compounds and/or intermediates were characterized by high performance liquid chromatography (HPLC) using a Waters ACQUITY UPLC system with a SQ (single quadrupole) MS and a photodiode array (PDA) detector (Milford, Mass.). The analytical columns were reversed phase Acquity UPLC BEH C18 (2.1×50 mm, 1.7 μm). A gradient elution was used (flow 0.4 mL/min), typically starting with mobile phase 0.1% formic acid in water (solvent A) and 0.1% formic acid in acetonitrile (solvent B). A gradient starting at 95% solvent A going to 5% in 1.8 min., holding for 0.5 min., going back to 95% in 0.5 min. and equilibrating the column for 0.5 min. Compounds were detected by ultraviolet light (UV) absorption at either 220 or 254 nm. HPLC solvents were from Burdick and Jackson (Muskegan, Mich.), or Fisher Scientific (Pittsburgh, Pa.).

In some instances, purity was assessed by thin layer chromatography (TLC) using glass or plastic backed silica gel plates, such as, for example, Baker-Flex Silica Gel IB2-F flexible sheets. TLC results were readily detected visually under ultraviolet light, or by employing well-known iodine vapor and other various staining techniques

The compounds and/or intermediates were characterized by LCMS. General conditions are as follows. Low and High resolution Mass spectra were acquired on LC/MS systems using electrospray ionization methods from a range of instruments of the following configurations: Low resolution—Waters ACQUITY UPLC system with a SQ (single quadrupole) MS; Waters ACQUITY UPLC H-Class system with a 3100 (single quadrupole) MS. High resolution—Waters ACQUITY UPLC II system equipped with a Synapt Xevo QTof and Waters ACQUITY UPLC II system equipped with a Synapt G2S QTof mass spectrometer with an atmospheric pressure ionization source. [M+H] refers to the protonated molecular ion of the chemical species.

Nuclear magnetic resonance (NMR) analysis was performed on a Bruker 500 MHz NMR spectrometer using ICON-NMR, under TopSpin program control. Spectra were measured at 298K, unless indicated otherwise and were referenced relative to the solvent chemical shift.

### B. Synthesis of Compounds of the Application

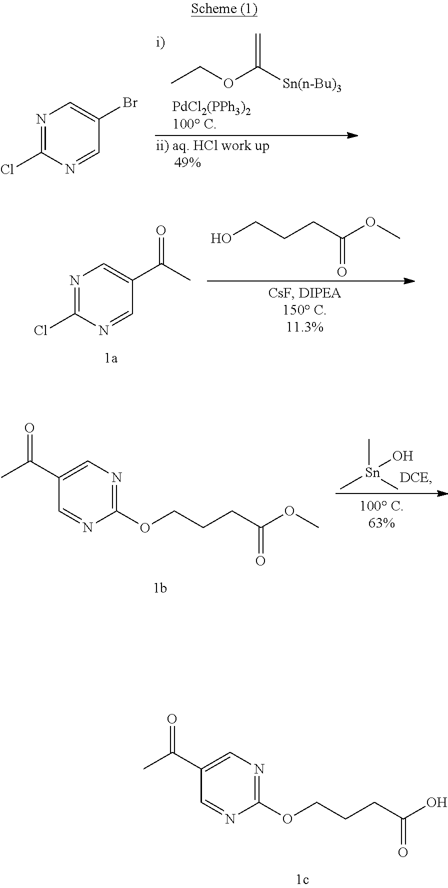

### 1-(2-Chloropyrimidin-5-yl) ethan-1-one (1a)

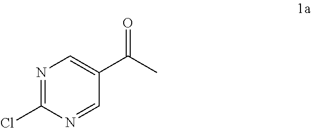

To a stirred solution of 5-bromo-2-chloropyrimidine (1 g, 5.2 mmol) in DMF was added tributyl(1-ethoxyvinyl)stannane (1.86 g, 5.2 mmol) at RT followed by addition of PdCl₂(PPh₃)₂ (0.18 g, 0.2 mmol) the mixture was heated at 100° C. for 3 h then stirred at RT for 16 h. The reaction mixture was quenched with a KF solution (50 mL) and extracted with EtOAc (2×50 mL). The combined organic layers were washed with water (50 mL), brine (50 mL), dried over Na₂SO₄ and concentrated down. The crude compound was acidified with 2N HCl (100 mL) stirred for 1 h at RT then extracted with EtOAc (2×50 mL). The combined organic layers were washed with water (50 mL), brine (50 mL) and dried over Na₂SO₄. It was concentrated under reduced pressure to give the crude compound. It was purified by column chromatography using silica gel (100-200 mesh) eluted with EtOAc in petroleum ether (0-30%) to afford compound 1a as a pale yellow semi-solid (400 mg, 49% yield). LCMS [M+H]⁺ 157.

### Methyl 4-((5-acetylpyrimidin-2-yl)oxy)butanoate (1b)

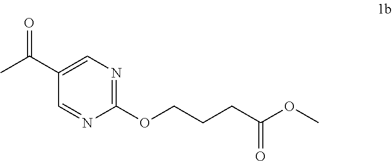 [SMILES: COC(=O)CCCOc1ncc(C(C)=O)cn1]

To a stirred solution of compound 1a (7 g, 44.8 mmol) in DMSO (150 mL), was added CsF (13.6 g, 89.7 mmol) and DIPEA (9.9 mL, 53.84 mmol) at RT followed by addition of methyl 4-hydroxybutanoate (10.58 g, 89.7 mmol). The reaction mixture was heated to 150° C. for 1 h before being cooled to RT. The reaction mixture was poured into ice-water (500 mL) and filtered through a celite bed which was washed with EtOAc. The filtrate was extracted with EtOAc (2×500 mL). The combined organic layers were washed with brine (200 mL) and dried over Na₂SO₄. It was concentrated and purified by column chromatography using silica gel (100-200 mesh) eluted with EtOAc in petroleum ether (0-30%) to afford compound 1b as a pale yellow semi-solid (1.2 g, 11.3% yield). LCMS [M+H]⁺ 239.

### 4-((5-Acetylpyrimidin-2-yl)oxy)butanoic acid (1c)

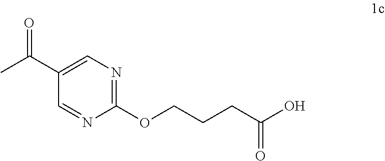 [SMILES: CC(=O)c1cnc(OCCCC(=O)O)nc1]

To a stirred solution of compound 1b (1 g, 4.2 mmol) in DCE (50 mL) was added trimethyl tin hydroxide (5.86 g, 21 mmol). The reaction mixture was heated to 100° C. for 48 h before being cooled to RT. It was concentrated under reduced pressure then was basified with aq NaHCO₃. It was washed with DCM (2×100 mL). The aqueous layer was acidified with 2N HCl (20 mL), extracted with DCM (2×100 mL). The combined organic layers were washed with water (50 mL), brine (50 mL) and dried over Na₂SO₄. It was concentrated to afford a solid which was washed with n-pentane (5 mL) to afford compound 1c as an off-white solid (0.6 g, 63% yield). ¹H NMR (400 MHz, DMSO-d₆): δ12.04 (bs, 1H), δ 9.07 (s, 2H), δ 4.43 (t, J=6.4 Hz, 1H), δ 2.57 (s, 3H), δ 2.40 (t, J=6.4 Hz, 2H), δ 2.03-1.94 (m, 2H); LCMS [M+H]⁺ 225.

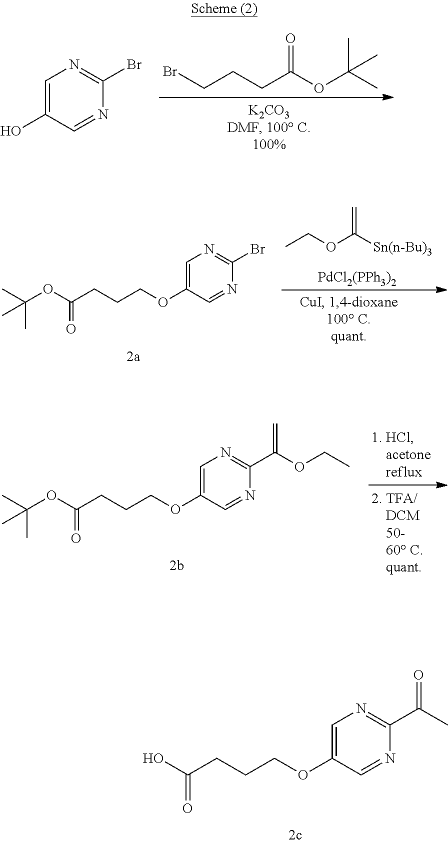

### tert-Butyl 4-((2-bromopyrimidin-5-yl)oxy)butanoate (2a)

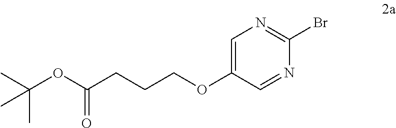 [SMILES: CC(C)(C)OC(=O)CCCOc1cnc(Br)nc1]

A 250 mL RB flask was charged with 2-bromo-5-hydroxypyrimidine (2 g, 11.43 mmol), t-butyl 4-bromobutanoate (3.06 g, 13.72 mmol) and potassium carbonate (1.896 g, 13.72 mmol) then N,N-dimethylformamide (15 mL) was added. The mixture was heated at 100° C. for 2 h upon which LCMS showed completion. The mixture was cooled down then a large volume of water was added followed by EtOAc. The organic layer was washed several times with water to remove the DMF then with brine. It was dried over Na₂SO₄ then concentrated down to afford compound 2a as an oil that solidified to a brown solid (3.62 g, 100% yield). ¹H NMR (CHLOROFORM-d, 500 MHz) δ 8.27 (s, 2H), 4.12 (t, 2H, J=6.2 Hz), 2.45 (t, 2H, J=7.2 Hz), 2.12 (quin, 2H, J=6.7 Hz), 1.47 (s, 9H); LCMS [M+H]⁺ 317.

### tert-Butyl 4-((2-(1-ethoxyvinyl)pyrimidin-5-yl)oxy)butanoate (2b)

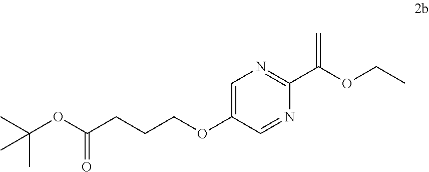 [SMILES: C=C(OCC)c1ncc(OCCCC(=O)OC(C)(C)C)cn1]

A small microwave vial was charged with compound 2a (316 mg, 0.996 mmol), 1-ethoxyvinyltri-n-butyltin (1079 mg, 2.99 mmol) and 1,4-dioxane (3 mL). It was degassed with a gentle stream of N₂ for few minutes upon which copper(I) iodide (28.5 mg, 0.149 mmol) and Pd(PPh₃)₂Cl₂ (35.0 mg, 0.050 mmol) were added. The vial was sealed and heated in the microwave at 100° C. under a high absorption mode. After 1 h, LCMS showed completion. The mixture was separated between water and EtOAc. An emulsion was formed. It was filtered through a short pad of Celite™ then washed with EtOAc. The organic layer was washed with brine then dried over MgSO₄. It was concentrated down onto celite then purified using CombiFlash RF (12 g silica column, eluent EtOAc/Hexanes 0%, 0-50% then 50%). This purification yielded the right product 2b in two fractions: a brown thick oil (209 mg, 68% yield) and a sticky brown gum which contained some tin by-products (105 mg, 34.2% yield). The overall yield is >100% because the second fraction contains some tin by-products. ¹H NMR (CHLOROFORM-d, 500 MHz) δ 8.34 (s, 2H), 5.45 (d, 1H, J=2.1 Hz), 4.46 (d, 1H, J=2.1 Hz), 4.04 (t, 2H, J=6.2 Hz), 3.96 (q, 2H, J=7.0 Hz), 2.37 (t, 2H, J=7.2 Hz), 2.04 (quin, 2H, J=6.7 Hz), 1.43 (t, 3H, J=7.0 Hz), 1.38 (s, 9H); LCMS [M+H]⁺ 309.

### 4-((2-Acetylpyrimidin-5-yl)oxy)butanoic acid (2c)

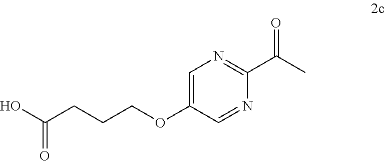 [SMILES: CC(=O)c1ncc(OCCCC(=O)O)cn1]

Compound 2b (206 mg, 0.668 mmol) was dissolved in acetone (10 mL) then HCl (1 M) (3 mL) was added. The mixture was heated to reflux for about 30 min upon which LCMS showed that only the enol ether had hydrolyzed. The solvents were evaporated down and the flask was dried with pressurized air. The residue was taken into DCM/TFA 3 mL/3 mL and heated between 50-60° C. After 1 h, LCMS showed completion of the reaction. The mixture was concentrated then dried under high vacuum to afford crude compound 2c as a yellow to orange solid (154 mg, quant.). The yield was >100% because there was some residual TFA in the product. ¹H NMR (DMSO-d6, 500 MHz) δ 11.8-12.4 (m, 1H), 8.70 (s, 2H), 4.28 (t, 1H, J=6.5 Hz), 2.64 (s, 3H), 2.42 (t, 2H, J=7.3 Hz), 2.00 (quin, 2H, J=6.8 Hz); LCMS [M+H]⁺ 225.

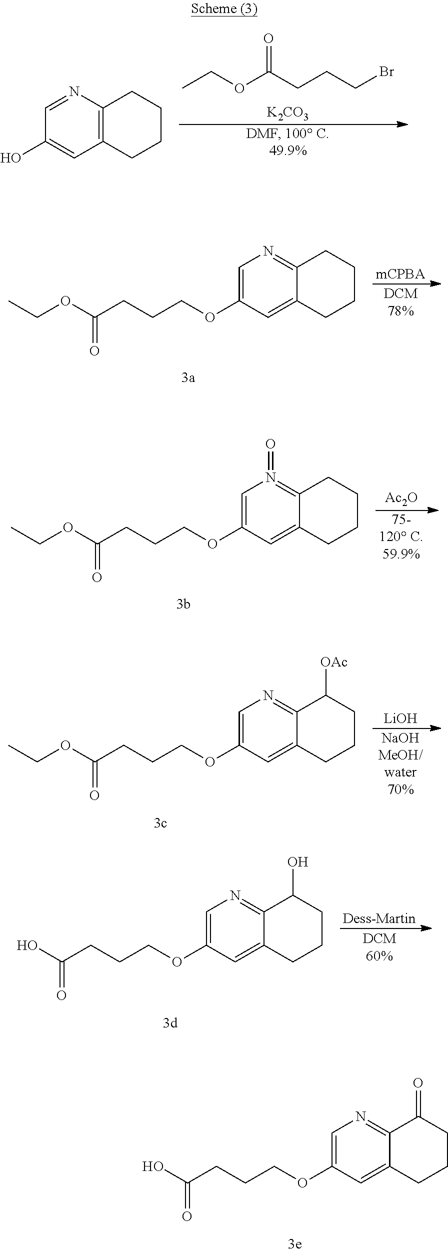

### Ethyl 4-((5,6,7,8-tetrahydroquinolin-3-yl)oxy)butanoate (3a)

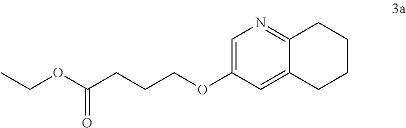 [SMILES: CCOC(=O)CCCOc1cnc2c(c1)CCCC2]

A 250 mL RB flask was charged with 5,6,7,8-tetrahydroquinolin-3-ol (3.25 g, 21.78 mmol) and N,N-dimethylformamide (15 mL). Potassium carbonate (3.61 g, 26.1 mmol) was added followed by ethyl 4-bromobutyrate (3.12 mL, 21.78 mmol). The mixture was heated at 100° C. After about 3 h, K₂CO₃ (3.8 g) and ethyl 4-bromobutyrate (3.3 mL). The mixture was heated at 100° C. for 1 h then was stirred at RT overnight. A large volume of water was added followed by EtOAc. The organic layer was washed several times with water to remove the DMF then brine. It was dried over Na₂SO₄ then concentrated down. It was purified using CombiFlash RF (24 g silica column, eluent EtOAc/hexanes 0-100% then 100%) to afford compound 3a as a dark orange liquid (2.86 g, 49.9% yield). LCMS [M+H]⁺ 264.

### 3-(4-Ethoxy-4-oxobutoxy)-5,6,7,8-tetrahydroquinoline 1-oxide (3b)

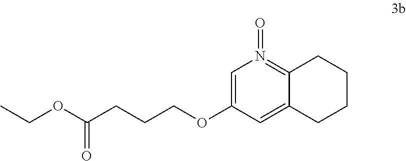 [SMILES: CCOC(=O)CCCOc1cc2c([n+]([O-])c1)CCCC2]

Compound 3a (2.85 g, 10.82 mmol) was dissolved in dry DCM (25 mL) then mCPBA (3.74 g, 21.65 mmol) was added. The mixture was stirred at RT for 1 h upon which LCMS showed completion. The reaction was still stirred overnight at RT. It was washed 4 times with saturated solution of sodium bicarbonate. In between washes, it was diluted with some DCM. The organic layer was dried over Na₂SO₄, concentrated down and dried under high vacuum to afford the crude compound 3b as a light brown solid (2.36 g, 78% yield). LCMS [M+H]⁺ 280.

### Ethyl 4-((8-acetoxy-5,6,7,8-tetrahydroquinolin-3-yl)oxy)butanoate (3c)

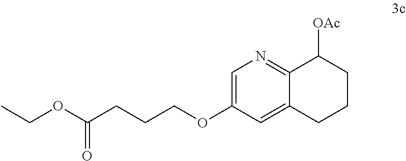 [SMILES: CCOC(=O)CCCOc1cnc2c(c1)CCCC2OC(C)=O]

Compound 3b (2.355 g, 8.43 mmol) was suspended in acetic anhydride (11.95 ml, 126 mmol) then the mixture was stirred in a preheated oil bath at 75° C. at which point it became a clear solution. After about 1 h, it was heated to 120° C. for another hour. The solvent was removed under high vacuum. The residue was taken in DCM, washed with water then with a saturated solution of sodium bicarbonate. It was dried over Na₂SO₄ and concentrated down. The crude was purified using CombiFlash RF (24 g silica column, eluent EtOAc/hexanes 0-40% then 40%). The right product 3c was collected as a light brown oil (1.624 g, 59.9% yield). LCMS [M+H]⁺ 322.

### 4-((8-Hydroxy-5,6,7,8-tetrahydroquinolin-3-yl)oxy)butanoic acid (3d)

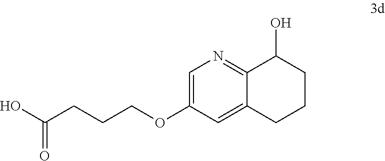 [SMILES: O=C(O)CCCOc1cnc2c(c1)CCCC2O]

Compound 3c (1.602 g, 4.98 mmol) was dissolved in MeOH (10 mL) then it was treated with a solution of lithium hydroxide monohydrate (0.209 g, 4.98 mmol) in water (3.33 mL) and stirred at RT. After about 2 h, only a very slow conversion was observed. NaOH (9 mL, 1 N) was added in 3 portions over 40 min. It was stirred for an additional 30 min upon which LCMS showed completion. The volatiles were removed under vacuum. The residual solution was cooled to 0° C. then treated with HCl (1 N) to pH (5-6). It was extracted once with DCM but most of the product remained in the aqueous layer. Both layers were loaded onto celite and dried. It was purified using CombiFlash RF (13 g C18 column: eluent AcCN/water 10%, 10-40% then 40%). The right compound 3d was collected as a light brown to a beige solid (877 mg, 70% yield). ¹H NMR (DMSO-d6, 500 MHz) δ 8.08 (d, 1H, J=2.7 Hz), 7.09 (d, 1H, J=2.7 Hz), 4.51 (t, 1H, J=4.5 Hz), 4.01 (t, 2H, J=6.7 Hz), 2.7-2.8 (m, 1H), 2.6-2.7 (m, 1H), 2.20 (t, 2H, J=7.2 Hz), 1.8-1.9 (m, 5H), 1.6-1.7 (m, 1H); LCMS [M+H]⁺ 252.

### 4-((8-Oxo-5,6,7,8-tetrahydroquinolin-3-yl)oxy)butanoic acid (3e)

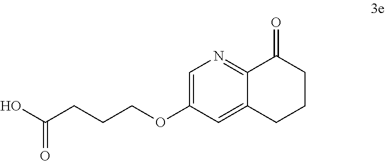 [SMILES: O=C(O)CCCOc1cnc2c(c1)CCCC2=O]

Compound 3d (780 mg, 3.10 mmol) was suspended in DCM (15 mL) then it was cooled to 0° C. Dess-Martin periodinane (1514 mg, 3.57 mmol) was added as a solid followed by a mixture of DCM (20 mL) and water (0.064 mL, 3.57 mmol). It was stirred at RT overnight. LCMS showed almost completion. It was cooled to 0° C. then quenched with MeOH. The mixture was loaded on celite and dried. It was purified using CombiFlash RF (24 g silica column eluent MeOH/DCM 0-5% then 5%). The right product 3e was collected as a light yellow solid (464 mg, 60% yield). ¹H NMR (DMSO-d6, 500 MHz) δ 12.0-12.4 (m, 1H), 8.28 (d, 1H, J=2.8 Hz), 7.35 (d, 1H, J=2.7 Hz), 4.15 (t, 2H, J=6.4 Hz), 2.98 (t, 2H, J=6.0 Hz), 2.6-2.7 (m, 2H), 2.40 (t, 2H, J=7.3 Hz), 2.04 (td, 2H, J=6.4, 12.5 Hz), 1.98 (quin, 2H, J=6.9 Hz); LCMS [M+H]⁺ 250.

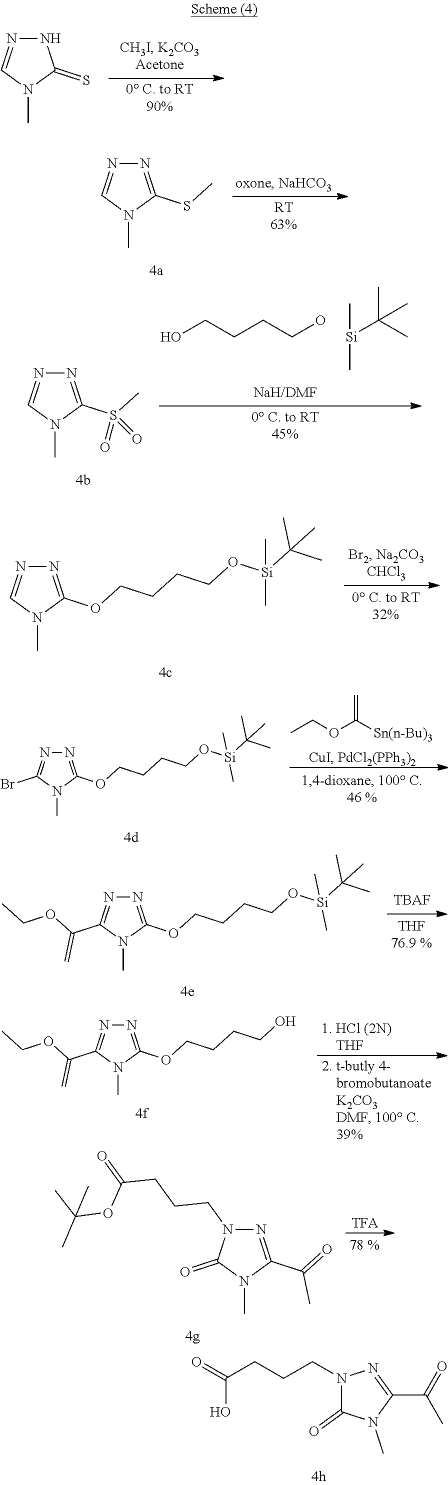

### 4-Methyl-3-(methylthio)-4H-1,2,4-triazole (4a)

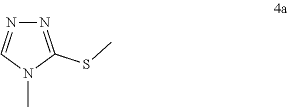

To a stirred solution of 4-methyl-2,4-dihydro-3H-1,2,4-triazole-3-thione (10 g, 86.95 mmol) in acetone (200 mL), methyl iodide (5.4 mL, 86.9 mmol) and K₂CO₃ (13.1 g, 95.6 mmol) were added at 0° C. then the reaction mixture was stirred at RT for 6 h. The solvent was evaporated then the residue was dissolved in DCM (500 mL). The solids were filtered through a pad of celite. The filtrate was concentrated under reduced pressure to afford compound 4a as a colorless liquid (10 g, 90% yield). ¹H NMR (CDCl3, 400 MHz) δ 8.13 (s, 1H), 3.58 (s, 3H), 2.75 (s, 3H).

### 4-Methyl-3-(methylsulfonyl)-4H-1,2,4-triazole (4b)

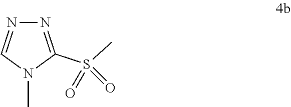

To a stirred solution of compound 4a (19 g, 147.2 mmol), in acetone: water (304 mL: 76 mL), oxone (135 g, 441.8 mmol) and NaHCO₃ (74 g, 883.6 mmol) were added then the reaction mixture was stirred at RT for 6 h. It was filtered through a celite bed and washed with acetone. The filtrate was concentrated then co-distilled with toluene to give the crude residue. It was dissolved in DCM (500 mL) and filtered to remove any solid. The filtrate was concentrated to afford compound 4b as a white solid (15 g, 63% yield). LCMS [M+H]⁺ 162.

### 3-(4-((tert-Butyldimethylsilyl)oxy)butoxy)-4-methyl-4H-1,2,4-triazole (4c)

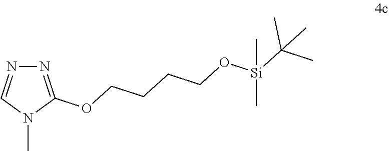 [SMILES: Cn1cnnc1OCCCCO[Si](C)(C)C(C)(C)C]

To a 0° C. cooled solution of 4-((tert-butyldimethylsilyl)oxy)butan-1-ol (4.7 g, 23.2 mmol) in DMF (25 mL) was added NaH (60%) (1.2 g, 31.0 mmol) at 0° C., then it was stirred for 30 min. Compound 4b (2.5 g, 15.5 mmol) was added. The reaction mixture was stirred at RT for 16 h then poured into ice water (500 mL) and extracted with EtOAc (2×300 mL). The combined organic layer was dried over Na₂SO₄ and concentrated under reduced pressure to afford crude compound 4c as a colorless liquid (2 g, 45% yield). TLC system: 20% EtOAc in petroleum ether; R_{f}: 0.5.

### 3-Bromo-5-(4-((tert-butyldimethylsilyl)oxy)butoxy)-4-methyl-4H-1,2,4-triazole (4d)

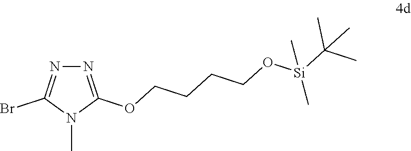 [SMILES: Cn1c(Br)nnc1OCCCCO[Si](C)(C)C(C)(C)C]

To a solution of compound 4c (3.4 g, 11.92 mmol) in DCM (60 mL), sodium carbonate (2.5 g, 23.84 mmol) and bromine (0.95 mL, 17.89 mmol) were added at 0° C. The mixture was stirred at RT for 5 h. It was, then, washed with a hypo solution, extracted with DCM (2×100 mL) and dried over sodium sulfate. After evaporating the solvent, the residue was purified by column chromatography (silica gel, 100-200 mesh, eluent EtOAc/Petroleum ether 40-50%) to afford compound 4d as a brown semi-solid (1.4 g, 32% yield). ¹H NMR (400 MHz, CDCl3) δ 4.47 (t, J=6.6 Hz, 2H), δ 3.66 (t, J=6.2 Hz 2H), δ 3.37 (s, 3H), δ 1.90-1.86 (m, 2H), δ 1.66-1.58 (m, 2H), δ 0.89 (s, 9H), δ 0.05 (s, 6H); LCMS [M+H]⁺ 364.

### 3-(4-((tert-Butyldimethylsilyl)oxy)butoxy)-5-(1-ethoxyvinyl)-4-methyl-4H-1,2,4-triazole (4e)

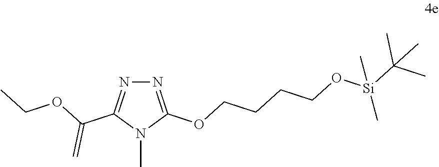 [SMILES: C=C(OCC)c1nnc(OCCCCO[Si](C)(C)C(C)(C)C)n1C]

A 20 mL microwave vial was charged with 3-bromo-5-(4-((tert-butyldimethylsilyl)oxy)butoxy)-4-methyl-4H-1,2,4-triazole 4d (1.2 g, 3.29 mmol), 1-ethoxyvinyltri-n-butyltin (3.57 g, 9.88 mmol) and 1,4-dioxane (12 mL). It was degassed with a gentle stream of N₂ for few minutes upon which copper (I) iodide (0.094 g, 0.494 mmol) and bis(triphenylphosphine)palladium(II) dichloride (0.116 g, 0.165 mmol) were added. The vial was sealed and heated in the microwave at 100° C. under a high absorption mode. After 1 h 15 min, LCMS showed still some SM left. 1-Ethoxyvinyltri-n-butyltin (1.1 g) was added then the vial was heated for 20 min at 100° C. The solvent was evaporated down. The mixture was dissolved in some DCM, loaded onto celite and dried. It was purified using CombiFlash RF (24 g silica column, eluent EtOAc/hexanes 0%, 0-50% then 50%). The desired product 4e was collected as a light yellow semi-solid (539 mg, 46% yield). ¹H NMR (CHLOROFORM-d, 500 MHz) δ 4.95 (d, 1H, J=2.7 Hz), 4.43 (t, 2H, J=6.5 Hz), 4.37 (d, 1H, J=2.7 Hz), 3.86 (q, 2H, J=7.0 Hz), 3.61 (t, 2H, J=6.3 Hz), 3.40 (s, 3H), 1.8-1.9 (m, 2H), 1.6-1.6 (m, 2H), 1.33 (t, 3H, J=7.0 Hz), 0.84 (s, 9H), 0.00 (s, 6H); LCMS [M+H]⁺ 356.

### 4-((5-(1-Ethoxyvinyl)-4-methyl-4H-1,2,4-triazol-3-yl)oxy)butan-1-ol (4f)

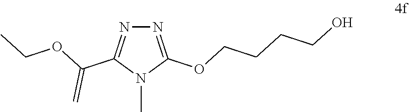 [SMILES: C=C(OCC)c1nnc(OCCCCO)n1C]

Compound 4e (556 mg, 1.564 mmol) was dissolved in THF (15 mL) then tetrabutylammonium fluoride 1 M in THF (4.69 mL, 4.69 mmol) was added. The mixture was stirred at RT. After 45 min, LCMS showed completion. The solvent was evaporated down. The residue was taken in EtOAc and washed with water (×7). However, all the product went into the aqueous layer. The water was evaporated and the crude was loaded onto celite and dried. It was purified using CombiFlash RF (13 g C18 column, eluent CH₃CN/H₂O: 0%, 0-50% then 50%). Most of the product eluted with the solvent front along with the TBAF. A later fraction eluted with minimal TBAF. It was collected as a light brown thick oil (141 mg). The front fraction was repurified using CombiFlash RF (24 g silica column, EtOAc/hexanes 0%, 0-100%, then 0-10% then acetone/EtOAc 10%) to afford the second fraction as a light yellow thick oil (149 mg). The overall yield of compound 4f was 76.9%. LCMS [M+H]⁺ 242.

### tert-Butyl 4-(3-acetyl-4-methyl-5-oxo-4,5-dihydro-1H-1,2,4-triazol-1-yl)butanoate (4g)

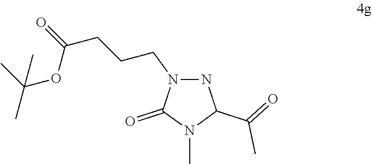 [SMILES: CC(=O)C1NN(CCCC(=O)OC(C)(C)C)C(=O)N1C]

4-((5-(1-ethoxyvinyl)-4-methyl-4H-1,2,4-triazol-3-yl)oxy)butan-1-ol 4f (140 mg, 0.580 mmol) was dissolved in THF (5 mL) then HCl (2 N, 5 mL) was added. The mixture was stirred at RT for 3 h upon which LCMS showed completion. The solvents were removed under vacuum and the residue was dried under high vacuum to afford the intermediate product as an orange solid (118 mg, the side chain having butanol was lost during this step). This intermediate was charged in 100 mL RB flask then t-butyl 4-bromobutanoate (111 mg, 0.496 mmol), potassium carbonate (82 mg, 0.595 mmol) and N,N-dimethylformamide (3 mL) were added. The mixture was heated at 100° C. for 1 h upon which LCMS showed completion. The mixture was cooled down and was diluted with EtOAc then water. The organic layer was washed twice with water to remove the DMF then brine. It was dried over MgSO₄. It was concentrated down and purified using CombiFlash RF (12 g silica column, eluent EtOAc/hexanes 0%, 0-50% then 50%) to afford the right product 4g as a very light yellow thick oil (65 mg, 39% yield). ¹H NMR (CHLOROFORM-d, 500 MHz) δ 3.95 (t, 2H, J=7.0 Hz), 3.57 (s, 3H), 2.55 (s, 3H), 2.3-2.4 (m, 2H), 2.09 (quin, 2H, J=7.2 Hz), 1.47 (s, 9H); LCMS [M+H-tBu]⁺ 228.

### 4-(3-Acetyl-4-methyl-5-oxo-4,5-dihydro-1H-1,2,4-triazol-1-yl)butanoic acid (4h)

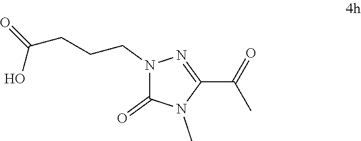 [SMILES: CC(=O)c1nn(CCCC(=O)O)c(=O)n1C]

tert-Butyl 4-(3-acetyl-4-methyl-5-oxo-4,5-dihydro-1H-1,2,4-triazol-1-yl)butanoate 4g (63 mg, 0.222 mmol) was dissolved in DCM (1 mL) then trifluoroacetic acid (1 mL) was added. The mixture was stirred at RT for 30 min. The volatiles were removed. The residue was dried under high vacuum to afford compound 4h as an off-white solid (59 mg, 78% yield). ¹H NMR (DMSO-d6, 500 MHz) δ 12.14 (br s, 1H), 3.84 (t, 2H, J=6.9 Hz), 3.38 (s, 3H), 2.45 (s, 3H), 2.29 (t, 2H, J=7.3 Hz), 1.90 (quin, 2H, J=7.1 Hz); LCMS [M+H]⁺ 228.

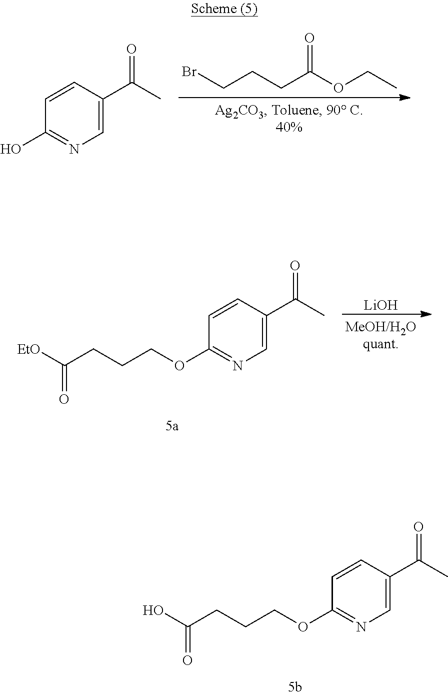

### Ethyl 4-((5-acetylpyridin-2-yl)oxy)butanoate (5a)

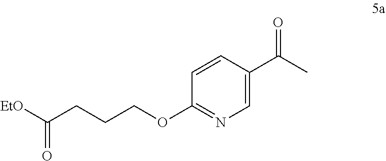 [SMILES: CCOC(=O)CCCOc1ccc(C(C)=O)cn1]

To a reaction vial was charged with 2-hydroxy-5-acetylpyridine (440 mg, 3.21 mmol) in anhydrous toluene (10 mL) was added silver carbonate (973 mg, 3.53 mmol) followed by ethyl 4-bromobutyrate (0.689 ml, 4.81 mmol). The mixture was heated at 90° C. for 3 days. Additional silver carbonate (531 mg, 1.925 mmol) was added followed by ethyl 4-bromobutyrate (0.344 ml, 2.406 mmol). The reaction was stirred at 90° C. over the weekend. The reaction was poured into a mixture of water and DCM and the organic layer separated, dried over MgSO₄ and concentrated onto celite. The material was purified on the Biotage (silica gel) eluting with 0-50% EtOAc/Hexanes. The desired fractions were collected, concentrated and dried under high vacuum to afford 5a (320 mg, 39.7% yield) as a colorless oil. ¹H NMR (500 MHz, CHLOROFORM-d) δ=8.76 (s, 1H), 8.15 (br d, J=8.7 Hz, 1H), 6.77 (d, J=8.7 Hz, 1H), 4.43 (t, J=6.2 Hz, 2H), 4.15 (br d, J=7.1 Hz, 2H), 2.57 (s, 3H), 2.50 (t, J=7.3 Hz, 2H), 2.14 (quin, J=6.8 Hz, 2H), 1.28-1.26 (m, 3H); LCMS [M+H]⁺ 252.

### 4-((5-Acetylpyridin-2-yl)oxy)butanoic acid (5b)

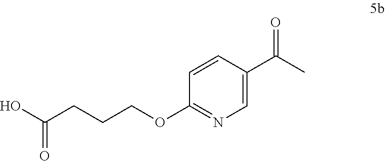 [SMILES: CC(=O)c1ccc(OCCCC(=O)O)nc1]

To a solution of 5a (320 mg, 1.273 mmol) in methanol (5.0 ml) was added a solution of lithium hydroxide monohydrate (107 mg, 2.55 mmol) in water (2.5 mL) dropwise. The reaction was stirred at RT overnight. The volatiles were removed under vacuum then the aqueous layer was acidified with 1N HCl. The reaction was concentrated under vacuum, followed by lyophilization to afford 5b (353 mg, >100% yield because it contains LiCl salt) as a pale yellow solid (¹H NMR (500 MHz, DMSO-d6) δ=8.80 (s, 1H), 8.17 (br d, J=8.7 Hz, 1H), 6.90 (d, J=8.7 Hz, 1H), 4.35 (br t, J=6.5 Hz, 2H), 2.55 (s, 3H), 2.37 (brt, J=7.3 Hz, 2H), 1.96 (quin, J=6.8 Hz, 2H); LCMS [M+H]⁺ 224.

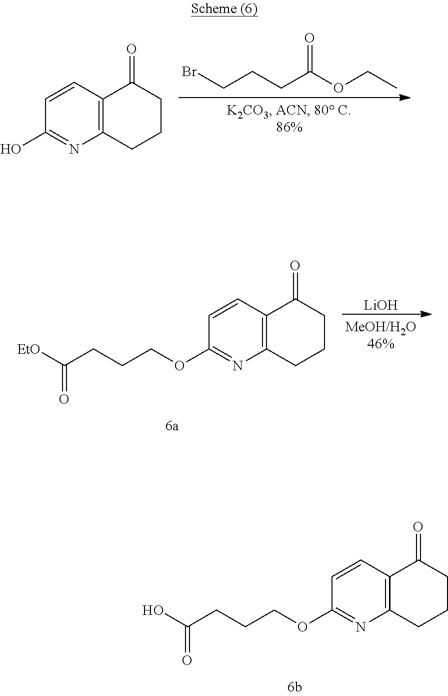

### Ethyl 4-((5-oxo-5,6,7,8-tetrahydroquinolin-2-yl)oxy)butanoate (6a)

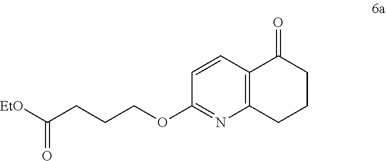 [SMILES: CCOC(=O)CCCOc1ccc2c(n1)CCCC2=O]

To a round bottom flask charged with 2-hydroxy-7,8-dihydroquinolin-5(6H)-one (1.0 g, 6.13 mmol) in acetonitrile (25 mL) was added potassium carbonate (0.932 g, 6.74 mmol) followed by ethyl 4-bromobutyrate (0.965 ml, 6.74 mmol). The mixture was heated at 80° C. overnight. LCMS showed a mixture of N-alkylated (16%) and O-alkylated (84%) products. The reaction was concentrated under vacuum onto celite and purified on the Biotage (silica gel) eluting with 0-40% EtOAc/Hexanes. The desired fractions were collected, concentrated and dried under high vacuum at RT to afford 6a as a colorless oil (1.47 g, 86% yield). LCMS [M+H]⁺ 278.

### 4-((5-Oxo-5,6,7,8-tetrahydroquinolin-2-yl)oxy)butanoic acid (6b)

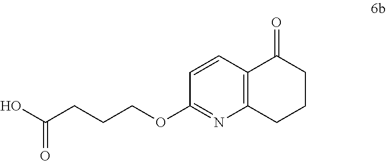 [SMILES: O=C(O)CCCOc1ccc2c(n1)CCCC2=O]

To a solution of 6a (1.47 g, 5.30 mmol) in methanol (24 mL) was added a solution of lithium hydroxide monohydrate (0.445 g, 10.60 mmol) in water (12 mL) dropwise. The reaction was stirred at RT overnight. The volatiles were removed under vacuum then the aqueous layer was acidified with 1N HCl. The acidic aqueous layer was washed with DCM (2×), dried over MgSO₄, concentrated and dried on the high vacuum overnight to 6b as a white solid (612 mg, 46.2% yield). ¹H NMR (500 MHz, CHLOROFORM-d) δ ppm 8.17 (br d, J=8.56 Hz, 1H), 6.64 (br d, J=8.56 Hz, 1H), 4.45 (br t, J=5.99 Hz, 2H), 3.00 (br t, J=5.99 Hz, 2H), 2.63 (br t, J=6.36 Hz, 2H), 2.57 (br t, J=7.15 Hz, 2H), 2.11-2.19 (m, 4H), LCMS [M+H]⁺ 250.38

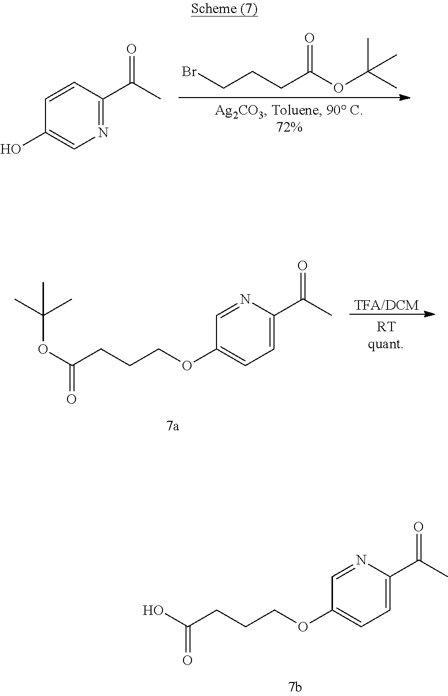

### tert-Butyl 4-((6-acetylpyridin-3-yl)oxy)butanoate (7a)

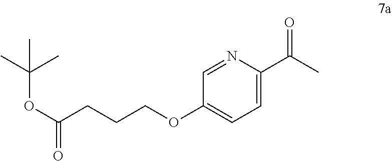 [SMILES: CC(=O)c1ccc(OCCCC(=O)OC(C)(C)C)cn1]

To a reaction vial charged with 1-(5-hydroxypyridin-2-yl)ethanone (500 mg, 3.65 mmol) in anhydrous toluene (10 mL) was added silver carbonate (1508 mg, 5.47 mmol) followed by t-butyl 4-bromobutanoate (0.970 mL, 5.47 mmol). The reaction was heated at 90° C. overnight. Additional silver carbonate (1508 mg, 5.47 mmol) was added followed by t-butyl 4-bromobutanoate (0.970 mL, 5.47 mmol). The reaction was heated at 90° C. overnight. It was then poured into a mixture of water and DCM and the organic layer separated, dried over MgSO₄ and concentrated onto celite. The material was purified on the Biotage (silica gel) eluting with 0-60% EtOAc/Hexanes. The desired fractions were collected, concentrated and dried under high vacuum at RT to afford 7a as a pale yellow oil (729 mg, 72% yield). ¹H NMR (500 MHz, CHLOROFORM-d) δ ppm 8.33 (d, J=2.69 Hz, 1H), 8.06 (d, J=8.68 Hz, 1H), 7.27 (dd, J=8.80, 2.93 Hz, 1 H), 4.14 (t, J=6.17 Hz, 2H), 2.70 (s, 3H), 2.47 (t, J=7.15 Hz, 2H), 2.14 (t, J=6.54 Hz, 2H), 1.48 (s, 9H); LCMS [M+H]⁺ 280.

### 4-((6-Acetylpyridin-3-yl)oxy)butanoic acid (7b)

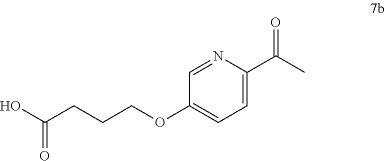 [SMILES: CC(=O)c1ccc(OCCCC(=O)O)cn1]

To a solution of 7a (729 mg, 2.61 mmol) in dichloromethane (2 mL) was added trifluoroacetic acid (1.998 mL, 26.1 mmol). The reaction was heated at 40° C. over the weekend. It was then concentrated under vacuum, taken up in water and lyophilized to afford 7b as a white solid (669 mg, 76% yield, TFA salt). ¹H NMR (500 MHz, DMSO-d6) δ ppm 12.04-12.40 (m, 1H), 8.39 (d, J=2.57 Hz, 1H), 7.96 (d, J=8.80 Hz, 1H), 7.54 (dd, J=8.74, 2.87 Hz, 1H), 4.17 (t, J=6.48 Hz, 2H), 2.59 (s, 3H), 2.41 (t, J=7.27 Hz, 2H), 1.99 (br t, J=6.85 Hz, 2H); LCMS [M+H]⁺ 224.

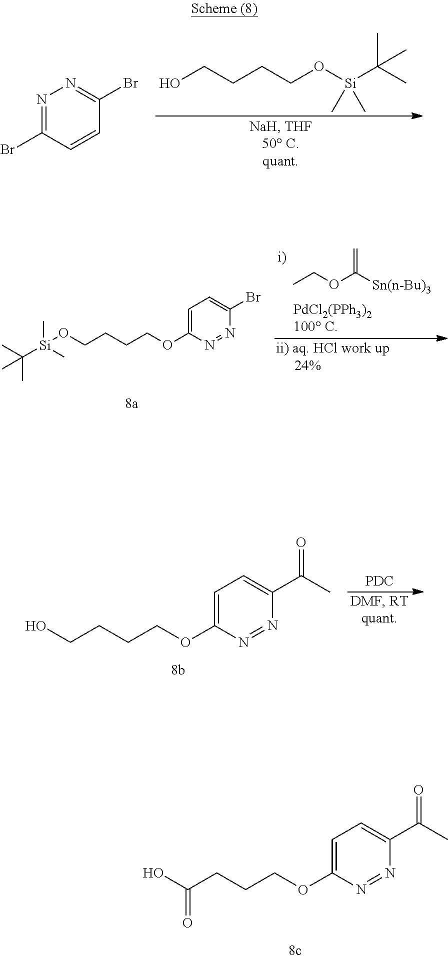

### 3-Bromo-6-(4-((tert-butyldimethylsilyl)oxy)butoxy)pyridazine (8a)

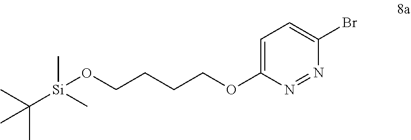 [SMILES: CC(C)(C)[Si](C)(C)OCCCCOc1ccc(Br)nn1]

To a 0° C. solution of 4-[(tert-butyldimethylsilyl)oxy]butan-1-ol (1.89 g, 9.25 mmol) in anhydrous tetrahydrofuran (20 mL) was added sodium hydride, 60% in mineral oil (1.68 g, 42 mmol) and then the mixture was stirred for 1 hour. 3,6-Dibromopyridazine (2 g, 8.41 mmol) was added portionwise at 0° C. then warmed to RT for 10 min. The mixture was stirred at 50° C. for 3 hours. The reaction was slowly quenched with water then concentrated under vacuum. It was then partitioned between water and ethyl acetate. The water layer was separated then the organic layer was washed with water followed by brine. The organic extract was dried over MgSO₄, concentrated under vacuum to obtain 8a as a brown semi-solid (3.1 g, quant.). ¹H NMR (500 MHz, DMSO-d6) δ ppm 7.83 (d, J=9.17 Hz, 1H), 7.19 (d, J=9.29 Hz, 1H), 4.38 (t, J=6.60 Hz, 2H), 3.61 (t, J=6.24 Hz, 2H), 1.73-1.82 (m, 2H), 1.54-1.60 (m, 2H), 0.83 (s, 9H), 0.00 (s, 6H); LCMS [M+H]⁺ 361.

### 1-(6-(4-Hydroxybutoxy)pyridazin-3-yl)ethan-1-one (8b)

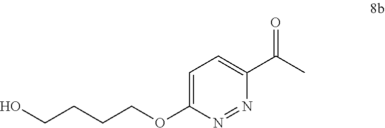

Bis(triphenylphosphine)palladium(II) dichloride (557 mg, 0.794 mmol) was added to a solution of 8a (2870 mg, 7.94 mmol) and 1-ethoxyvinyltri-n-butyltin (3729 mg, 10.33 mmol) in anhydrous tetrahydrofuran (50 mL). The flask was evacuated and backfilled with nitrogen twice, then heated at 70° C. overnight. The reaction mixture was quenched with 2M KF and extracted with EtOAc (×3). The combined organic layers were washed with 2M KF, brine, dried over MgSO₄, and concentrated onto celite. The crude material was purified on the Biotage eluting with 0-40% EtOAc/Hexanes. The desired fractions were collected, concentrated and dried under high vacuum at RT to afford 3-(4-((tert-butyldimethylsilyl)oxy)butoxy)-6-(1-ethoxyvinyl)pyridazine (1.52 g) as a yellow oil. To this intermediate (1.3 g, 3.69 mmol) in tetrahydrofuran (50 mL) was added 1N hydrochloric acid (55.3 mL, 55.3 mmol). The mixture was stirred at RT for 1 hour. The THF was removed under vacuum. The mixture was neutralized with sat NaHCO₃ (aq), extracted with DCM (2×), dried over MgSO₄ and concentrated under vacuum. The crude material was purified on the Biotage (reverse phase silica gel) eluting 0-50% ACN/H₂O. The desired fractions were collected, concentrated and dried under high vacuum at RT to afford 8b (356 mg, 29% overall yield) as a yellow residue. LCMS [M+H]⁺ 211.

### 4-((6-Acetylpyridazin-3-yl)oxy)butanoic acid (8c)

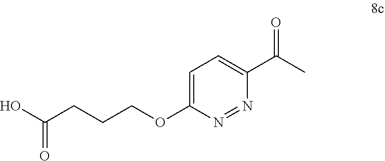 [SMILES: CC(=O)c1ccc(OCCCC(=O)O)nn1]

To a solution of 8b (356 mg, 1.693 mmol) in anhydrous N,N-dimethylformamide (10 mL) was added pyridinium dichromate (6371 mg, 16.93 mmol) and stirred at RT for 4 hours. The reaction mixture was poured into an aqueous saturated ammonium chloride solution, and extracted with DCM. The organic layer was further washed with water and brine, dried over MgSO₄ and concentrated to obtain 8c as a brown residue (380 mg, 100% yield). The crude material was carried onto the next step without further purification, quantitative yield assumed. LCMS [M+H]⁺ 225.

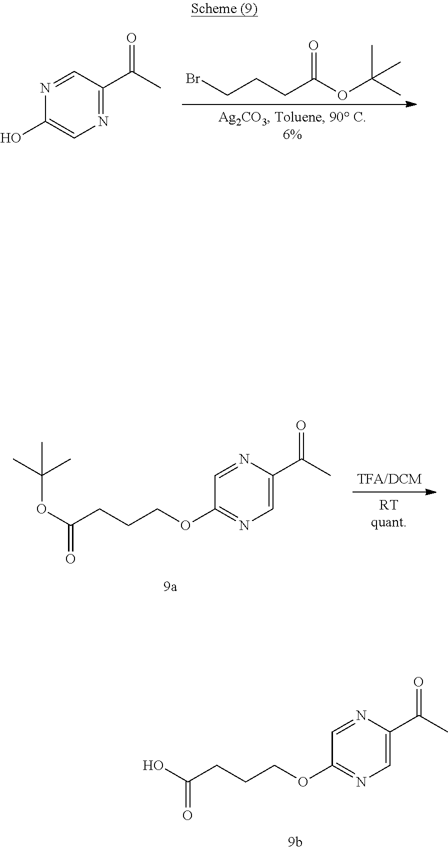

### tert-Butyl 4-((5-acetylpyrazin-2-yl)oxy)butanoate (9a)

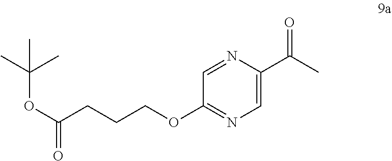 [SMILES: CC(=O)c1cnc(OCCCC(=O)OC(C)(C)C)cn1]

A vial was charged with 1-(5-hydroxypyrazin-2-yl)ethanone (994 mg, 7.20 mmol), anhydrous toluene (30 mL) and silver carbonate (3969 mg, 14.39 mmol). The mixture was stirred for 3 hours at RT followed by addition of t-butyl 4-bromobutanoate (1927 mg, 8.64 mmol). The mixture was heated at 90° C. overnight. Additional silver carbonate (3969 mg, 14.39 mmol) and t-butyl 4-bromobutanoate (1927 mg, 8.64 mmol) were added and further stirred at 90° C. overnight. LCMS showed ˜60% conversion with a mixture if C-alkylated and O-alkylated products. The reaction was poured into a mixture of water and DCM and the organic layer separated, dried over MgSO₄ and concentrated onto celite. The material was purified on the Biotage (silica gel) eluting with 0-50% EtOAc/Hexanes. The desired fractions were collected, concentrated and dried under high vacuum at RT to afford 9a as a yellow oil (121 mg, 6% yield). ¹H NMR (500 MHz, CHLOROFORM-d) δ ppm 8.72 (d, J=1.35 Hz, 1H), 8.11 (d, J=1.35 Hz, 1H), 4.38 (t, J=6.42 Hz, 2H), 2.58 (s, 3H), 2.34 (t, J=7.34 Hz, 2H), 2.00-2.06 (m, 2H), 1.38 (s, 9H); LCMS [M+H]⁺ 281.

### 4-((5-Acetylpyrazin-2-yl)oxy)butanoic acid (9b)

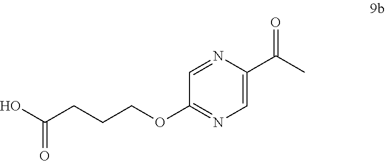 [SMILES: CC(=O)c1cnc(OCCCC(=O)O)cn1]

To a solution of 9a (121 mg, 0.432 mmol) in dichloromethane (2 mL) was added trifluoroacetic acid (0.331 mL, 4.32 mmol). The reaction was stirred at RT for 3 hours. It was concentrated under vacuum to obtain 9b as a yellow residue (97 mg, quant.) and carried onto next step without further purification. LCMS [M+H]⁺ 225.

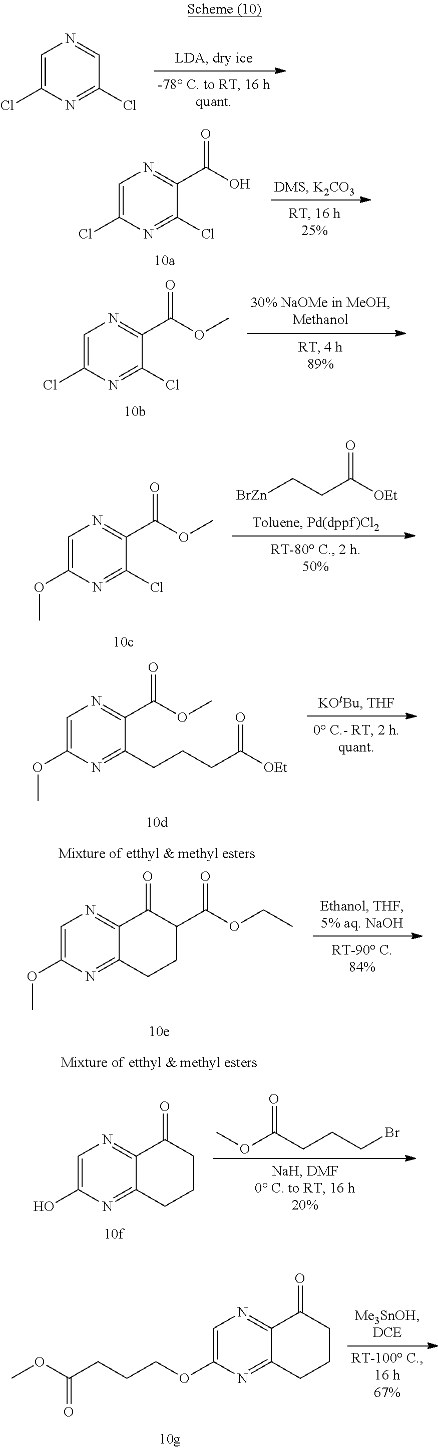

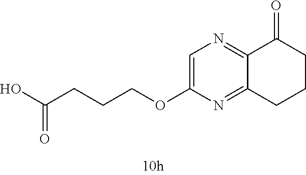 [SMILES: O=C(O)CCCOc1cnc2c(n1)CCCC2=O]

### 3,5-Dichloropyrazine-2-carboxylic acid (10a)

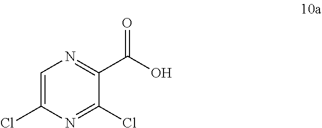 [SMILES: O=C(O)c1ncc(Cl)nc1Cl]

To a stirred solution of DIPA (59 mL, 419.4 mmol) in dry THF (1000 mL) was added n-BuLi (262 mL, 419.4 mmol, 1.6 M in hexanes) at −78° C. and allowed to warm to 0,−20° C. over 1 h. To this freshly prepared LDA was added a solution of 2,6-dichloropyrazine (25 g, 167.8 mmol) in dry THF (1000 mL) at −78° C. under argon atmosphere. The resulting mixture was stirred for 3 h at the same temperature. Dry ice (500 g) was added portionwise at −78° C. then it was allowed warm to RT over 16 h. The reaction mixture was quenched with water (500 mL) then acidified to pH˜ 2 with a saturated solution of NaHSO₄. It was extracted with EtOAc (3×500 mL). The combined organic layer was dried over Na₂SO₄ and concentrated under reduced pressure to afford the crude compound 10a (30 g, quant.) as a brown sticky solid. LCMS [M+H]⁺ 191.1 (about 52% pure)

### Methyl 3,5-dichloropyrazine-2-carboxylate (10b)

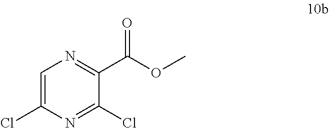

To a stirred solution of compound 10a (30 g (crude), 156.3 mmol) in acetone (300 mL) were added K₂CO₃ (32.34 g, 234.4 mmol) as a powder and dimethyl sulfate (23.6 g, 187.5 mmol) dropwise over a period of 30 min. The reaction mass was stirred at RT for 16 h. It was quenched with water (300 mL) and extracted with ethyl acetate (2×300 mL). The combined organic layers were washed with water (200 mL) and brine (200 mL). It was dried over Na₂SO₄, filtered and concentrated under vacuum. The crude product was purified by column chromatography using silica gel (100-200 mesh) eluted with 5% EtOAc in petroleum ether to afford compound 10b as an off-white solid (8 g, 2 step overall yield 25%). LCMS [M+H]⁺ 207.0.

### Methyl 3-chloro-5-methoxypyrazine-2-carboxylate (10c)

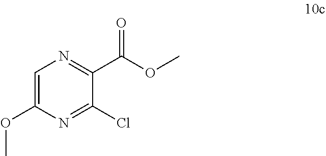

To a suspension of compound 10b (8 g, 38.3 mmol) in methanol (240 mL) was added 30% NaOMe (11.65 mL, 38.8 mmol) at RT. The reaction mixture was stirred at the same temperature for 4 h, quenched with ice-cold water (400 mL) then extracted with EtOAc (2×300 mL). The combined organic layers were washed with brine (300 mL), dried over Na₂SO₄ and concentrated under reduced pressure. The crude product was purified by column chromatography using silica gel (100-200 mesh) eluted with 10-12% EtOAc in petroleum ether to afford compound 10c as an off-white solid. (7 g, 89% yield). LCMS [M+H]⁺ 203.1.

### Methyl 3-(4-ethoxy-4-oxobutyl)-5-methoxypyrazine-2-carboxylate (10d)

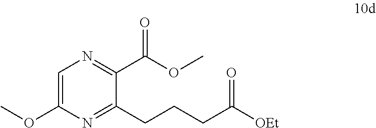 [SMILES: CCOC(=O)CCCc1nc(OC)cnc1C(=O)OC]

To a stirred solution of 10c (4 g, 19.8 mmol) in toluene (80 mL) was added (3-ethoxy-3-oxopropyl)zinc(II) bromide (59 mL, 29.7 mmol; 0.5M in toluene) dropwise over a period of 15 min at 15 to 25° C. followed by Pd(dppf)Cl₂.DCM (809 mg, 1 mmol). The reaction mixture was stirred under argon at 80° C. for 2 h then cooled to RT. It was quenched with ice cold water (200 mL), basified with a saturated NaHCO₃ solution (100 mL) and extracted with ethyl acetate (2×200 mL). The combined organic layers were concentrated under reduced pressure. The crude product was purified by column chromatography using silica gel (100-200 mesh) eluted with 20% EtOAc in petroleum ether to afford compound 10d as a pale yellow liquid (3 g, 50% yield). LCMS [M+H]⁺ 283.2, 297.2, 269.1 (might be a mixture of methyl and ethyl esters).

### Ethyl 2-methoxy-5-oxo-5,6,7,8-tetrahydroquinoxaline-6-carboxylate (10e)

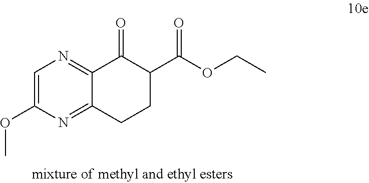 [SMILES: CCOC(=O)C1CCc2nc(OC)cnc2C1=O]

To a stirred solution of compound 10d (3 g, 10.6 mmol) in THF (60 mL) was added t-BuOK (16 mL, 16.0 mmol; 1.0M in THF) at 0° C. dropwise over a period of 15 min. The reaction mixture was stirred at RT for 2 h. It was quenched with ice cold water (50 mL) and extracted with ethyl acetate (3×50 mL). The combined organic layer was dried over Na₂SO₄ and concentrated under vacuum to afford Compound 10e as a yellow solid (1.8 g, quant). LCMS [M+H]⁺ 237.1, 251.1 (might be a mixture of methyl and ethyl esters).

### 2-Hydroxy-7,8-dihydroquinoxalin-5(6H)-one (10f)

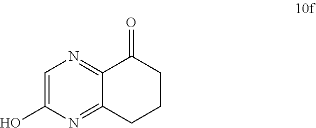 [SMILES: O=C1CCCc2nc(O)cnc21]

To a stirred solution of compound 10e (1.8 g, 7.2 mmol) in a solution of ethanol:THF:5% aq. NaOH (40 mL; 2:1:1) was added t-BuOK (16 mL, 16.0 mmol; 1.0 M in THF) at RT. The reaction mixture was stirred at 80° C. for 48 h, cooled to RT then concentrated under reduced pressure. The crude residue was quenched with ice cold water (50 mL) and extracted with 10% MeOH in DCM (2×100 mL). The combined organic layer was dried over Na₂SO₄ and concentrated under vacuum to afford compound 10f as a pale brown solid (1 g, 84% yield). LCMS [M+H]⁺ 165.1

### Methyl 4-((5-oxo-5,6,7,8-tetrahydroquinoxalin-2-yl)oxy)butanoate (10g)

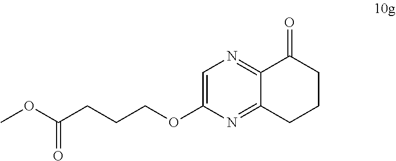 [SMILES: COC(=O)CCCOc1cnc2c(n1)CCCC2=O]

To a stirred solution of compound 10f (500 mg, 3.0 mmol) in DMF (20 mL) was added methyl 4-bromobutanoate (828 mg, 4.6 mmol) followed by NaH (134 mg, 3.3 mmol; 60% mineral oil) at 0° C. The reaction mixture was stirred at RT for 16 h. It was quenched with ice cold water (50 mL) and extracted with ethyl acetate (2×50 mL). The combined organic layer was washed with water (50 mL) and brine (50 mL). It was dried over Na₂SO₄ and concentrated under vacuum. The crude product was purified by column chromatography using silica gel (100-200 mesh) eluted with 40-50% EtOAc in petroleum ether to afford compound 10g as a pale brown liquid (200 mg, 20% yield). LCMS [M+H]⁺ 265.3.

### 4-((5-Oxo-5,6,7,8-tetrahydroquinoxalin-2-yl)oxy)butanoic acid (10h)

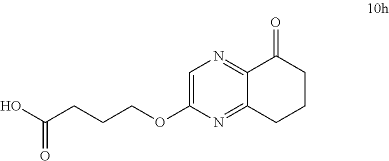 [SMILES: O=C(O)CCCOc1cnc2c(n1)CCCC2=O]

To a stirred solution of compound 10g (200 mg, 0.8 mmol) in DCE (10 mL) was added Me₃SnOH (685 mg, 3.8 mmol) at RT. The reaction mixture was stirred at 100° C. for 16 h, cooled to RT then concentrated under reduced pressure. The crude compound was stirred in a saturated NaHCO₃ solution (25 mL) for 30 min at RT and then washed with DCM (30 mL). The aqueous layer was acidified with a saturated NaHSO₄ solution to pH˜ 2 then extracted with ethyl acetate (2×30 mL). The combined organic layer was dried with Na₂SO₄ and concentrated under reduced pressure. The crude compound was purified by column chromatography using silica gel (100-200 mesh) eluting with 2-3% methanol in DCM to afford compound 10h as a pale brown solid (120 mg, 67% yield). ¹H NMR (400 MHz, DMSO-d₆): δ 12.19 (bs, 1H), 8.27 (s, 1H), 4.39 (t, J=6.4 Hz, 2H), 3.02 (t, J=6.0 Hz, 2H), 2.65 (t, J=6.6 Hz, 2H), 2.37 (t, J=7.2 Hz, 2H), 2.04-2.13 (m, 2H), 1.95-2.03 (m, 2H); LCMS [M+H]⁺ 251.1.

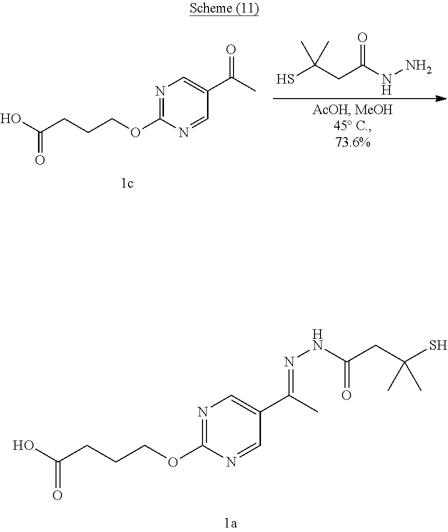

### 4-((5-(1-(2-(3-Mercapto-3-methylbutanoyl)hydrazineylidene)ethyl)pyrimidin-2-yl)oxy)butanoic acid (Ia)

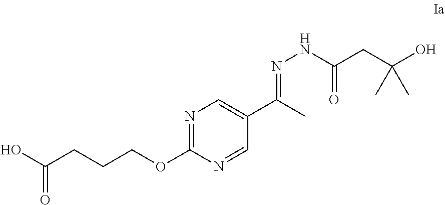 [SMILES: C/C(=N\NC(=O)CC(C)(C)O)c1cnc(OCCCC(=O)O)nc1]

A vial was charged with 4-((5-acetylpyrimidin-2-yl)oxy)butanoic acid 1c (250 mg, 1.115 mmol) and 3-mercapto-3-methylbutanehydrazide (198 mg, 1.338 mmol, some excess of the hydrazide was added). Methanol (1 mL) was added followed by acetic acid (447 μL, 7.80 mmol). The mixture was stirred at 50° C. for 5 h. It was loaded onto celite and dried. It was purified using CombiFlash RF (12 g silica column, eluent EtOAc/hexanes 0-100% then 100%). The right product Ia was collected as a beige solid (290.8 mg, 73.6% yield, 2 isomers). ¹H NMR (DMSO-d6, 500 MHz) δ 10.59 (s, 1H), 10.41 (s, 1H), 8.94 (s, 1H), 8.91 (s, 1H), 4.3-4.4 (m, 2H), 3.0-3.1 (m, 2H), 2.68 (s, 1H), 2.38 (br t, 2H, J=7.1 Hz), 2.34 (s, 1H), 2.27 (s, 1H), 2.24 (s, 1H), 1.97 (quin, 2H, J=6.9 Hz), 1.48 (br s, 3H), 1.47 (br s, 3H), 1.40 (s, 2H); LCMS [M+H]⁺ 355.

### 4-((2-(1-(2-(3-Mercapto-3-methylbutanoyl)hydrazineylidene)ethyl)pyrimidin-5-yl)oxy)butanoic acid (Ib)

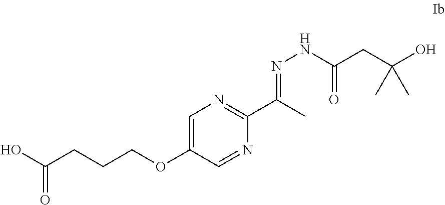 [SMILES: C/C(=N\NC(=O)CC(C)(C)O)c1ncc(OCCCC(=O)O)cn1]

Compound Ib was prepared using a similar procedure to compound Ia. It was collected as two fractions of several isomers. The first crop was collected as a beige solid (58 mg, 24% yield). ¹H NMR (DMSO-d6, 500 MHz) δ 13.76 (s, 1H), 13.35 (s, 1H), 11.9-12.4 (m, 1H), 10.56 (s, 1H), 10.34 (s, 1H), 8.81 (s, 1H), 8.79 (s, 1H), 8.61 (s, 1H), 8.59 (s, 1H), 4.2-4.3 (m, 1H), 4.2-4.2 (m, 1H), 3.0-3.1 (m, 2H), 3.02 (s, 1H), 2.72 (s, 1H), 2.62 (s, 1H), 2.4-2.4 (m, 2H), 2.38 (s, 1H), 2.34 (br d, 2H, J=10.5 Hz), 1.5-1.5 (m, 6H), 1.24 (s, 1H); LCMS [M+H]⁺ 355. A second fraction was collected as a mixture of 2 isomers (58 mg, 24% yield). ¹H NMR (DMSO-d6, 500 MHz) δ 11.7-12.6 (m, 1H), 10.56 (s, 1H), 10.34 (s, 1H), 8.61 (s, 1H), 8.59 (s, 1H), 4.2-4.2 (m, 2H), 3.0-3.1 (m, 2H), 2.72 (s, 1H), 2.41 (dt, 2H, J=3.2, 7.1 Hz), 2.35 (d, 1H, J=3.3 Hz), 2.33 (s, 1H), 1.99 (quin, 2H, J=6.5 Hz), 1.49 (br s, 3H), 1.48 (br s, 3H); LCMS [M+H]⁺ 355.

### 4-((8-(2-(3-Mercapto-3-methylbutanoyl)hydrazineylidene)-5,6,7,8-tetrahydroquinolin-3-yl)oxy)butanoic acid (Ic)

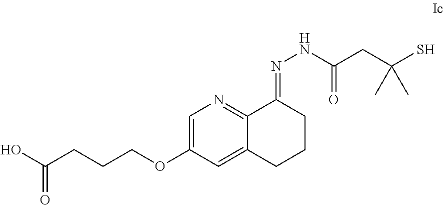 [SMILES: CC(C)(S)CC(=O)N/N=C1\CCCc2cc(OCCCC(=O)O)cnc21]

Compound Ic was prepared using a similar procedure to compound Ia. It was collected as a light brown gum (44.9 mg, 29.5% yield, mixture of isomers). 1H NMR (DMSO-d6, 500 MHz) δ 14.08 (s, 1H), 12.18 (br d, 1H, J=0.9 Hz), 9.99 (s, 1H), 9.91 (s, 1H), 8.35 (dd, 1H, J=2.8, 6.6 Hz), 7.4-7.5 (m, 1H), 4.16 (dt, 2H, J=2.8, 6.4 Hz), 4.04 (q, 1H, J=7.1 Hz), 3.0-3.1 (m, 4H), 2.9-2.9 (m, 3H), 2.6-2.7 (m, 2H), 2.40 (t, 2H, J=7.3 Hz), 2.00 (s, 2H), 1.97 (d, 1H, J=6.8 Hz), 1.92 (s, 3H), 1.91 (s, 2H), 1.85 (s, 3H), 1.84 (br s, 1H), 1.48 (s, 3H), 1.47 (s, 3H), 1.45 (s, 3H), 1.43 (s, 5H); LCMS [M+H]⁺ 380.

### 4-(3-(1-(2-(3-Mercapto-3-methylbutanoyl)hydrazineylidene)ethyl)-4-methyl-5-oxo-4,5-dihydro-1H-1,2,4-triazol-1-yl)butanoicacid (Id)

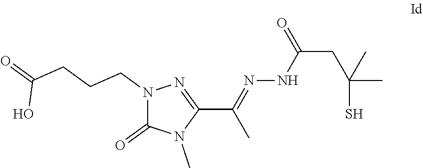 [SMILES: C/C(=N\NC(=O)CC(C)(C)S)c1nn(CCCC(=O)O)c(=O)n1C]

Compound Id was prepared using a similar procedure to compound Ia. It was collected as a white partially solidified gum (61.9 mg, 72.7% yield, mixture of isomers). 1H NMR (DMSO-d6, 500 MHz) δ 10.77 (s, 1H), 10.61 (s, 1H), 8.9-9.1 (m, 1H), 4.04 (q, 1H, J=7.1 Hz), 3.78 (t, 2H, J=6.8 Hz), 3.50 (s, 1H), 3.46 (s, 1H), 3.04 (s, 2H), 2.99 (s, 1H), 2.72 (s, 1H), 2.34 (s, 3H), 2.27 (t, 2H, J=7.2 Hz), 2.19 (d, 3H, J=6.8 Hz), 2.00 (s, 1H), 1.48 (br s, 3H), 1.48 (br s, 3H), 1.40 (s, 7H), 1.4-1.4 (m, 1H), 1.18 (t, 2H, J=7.2 Hz), LCMS [M+H]⁺ 358.

### 4-((5-(1-(2-(3-Mercapto-3-methylbutanoyl)hydrazineylidene)ethyl)pyridin-2-yl)oxy)butanoic acid (Ie)

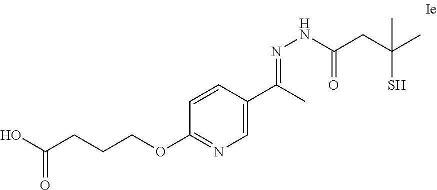 [SMILES: C/C(=N\NC(=O)CC(C)(C)S)c1ccc(OCCCC(=O)O)nc1]

The propyl ester of Ie was obtained using a similar procedure to compound Ia with propanol used as a solvent. However, during this reaction the acid was converted to the propyl ester. It was hydrolyzed back to the acid according to the following procedure: to a solution of the formed ester (58 mg, 0.147 mmol) in methanol (5.0 ml) was added a solution of lithium hydroxide monohydrate (12.31 mg, 0.293 mmol) in water (2.5 ml) dropwise. The reaction was stirred at RT overnight. The mixture was diluted with water and carefully acidified with 1N HCl (aq) using a pH meter until pH 6.5. The mixture was poured into a separatory funnel and washed with DCM (3×). The organic layers were combined, dried over MgSO₄, concentrated and dried under high vacuum to afford 10e as a colorless residue (25 mg, 5% yield). The crude material was used as-is in the next step. LCMS [M+H]⁺ 354.

### 4-((5-(2-(3-Mercapto-3-methylbutanoyl)hydrazineylidene)-5,6,7,8-tetrahydroquinolin-2-yl)oxy)butanoic acid (I)

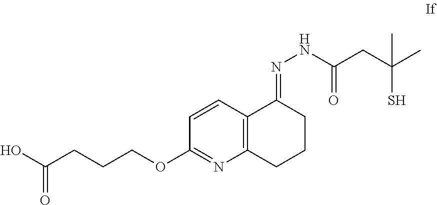 [SMILES: CC(C)(S)CC(=O)N/N=C1\CCCc2nc(OCCCC(=O)O)ccc21]

Compound If was prepared using a similar procedure to compound Ia. It was collected as an off-white solid (118 mg, 37% yield, mixture of isomers). ¹H NMR (500 MHz, DMSO-d6) δ=10.45-10.24 (m, 1H), 8.21 (br d, J=8.4 Hz, 1H), 6.71 (br t, J=10.1 Hz, 1H), 4.27 (br t, J=5.9 Hz, 2H), 3.04 (s, 2H), 2.81-2.73 (m, 2H), 2.63-2.57 (m, 2H), 2.38-2.34 (m, 2H), 1.93 (br t, J=6.8 Hz, 2H), 1.47 (br d, J=10.5 Hz, 6H), 1.39 (s, 3H); LCMS [M+H]⁺ 381.

### 4-((6-(1-(2-(3-Mercapto-3-methylbutanoyl)hydrazineylidene)ethyl)pyridin-3-yl)oxy)butanoic acid (Ig)

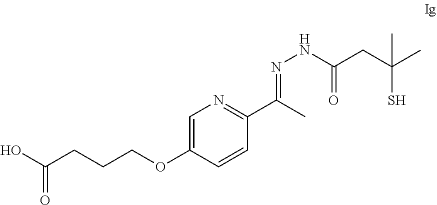 [SMILES: C/C(=N\NC(=O)CC(C)(C)S)c1ccc(OCCCC(=O)O)cn1]

Compound Ig was prepared using a similar procedure to compound Ia. It was collected as a white solid (191 mg, 91% yield, mixture of isomers). ¹H NMR (500 MHz, DMSO-d6) δ ppm 11.74-12.32 (m, 1H), 10.30-10.56 (m, 1H), 8.28 (br s, 1H), 8.00 (d, J=8.80 Hz, 1H), 7.46 (dt, J=8.50, 3.52 Hz, 1H), 4.11 (t, J=6.36 Hz, 2H), 3.02-3.11 (m, 2H), 2.70 (s, 1H), 2.40 (br t, J=7.15 Hz, 2H), 2.31 (d, J=13.94 Hz, 3H), 1.97 (quin, J=6.66 Hz, 2H), 1.49 (br d, J=8.44 Hz, 6H); LCMS [M+H]⁺ 354.

### 4-((6-(1-(2-(3-Mercapto-3-methylbutanoyl)hydrazineylidene)ethyl)pyridazin-3-yl)oxy)butanoic acid (Ih)

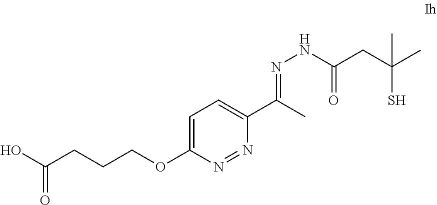 [SMILES: C/C(=N\NC(=O)CC(C)(C)S)c1ccc(OCCCC(=O)O)nn1]

Compound Ih was prepared using a similar procedure to compound Ia. It was collected as a yellow residue (139 mg, 24% yield, mixture of isomers). ¹H NMR (500 MHz, DMSO-d6) δ ppm 11.92-12.41 (m, 2H), 10.54-10.77 (m, 1H), 8.15 (dd, J=15.59, 9.35 Hz, 1H), 7.69 (s, 2H), 7.21-7.28 (m, 1H), 7.04 (s, 3H), 5.63-5.74 (m, 1H), 4.49 (t, J=6.48 Hz, 2H), 2.39-2.44 (m, 6H), 2.00-2.05 (m, 2H), 1.49 (d, J=5.01 Hz, 6H); LCMS [M+H]⁺ 355.

### 4-((5-(1-(2-(3-Mercapto-3-methylbutanoyl)hydrazineylidene)ethyl)pyrazin-2-yl)oxy)butanoic acid (Ii)

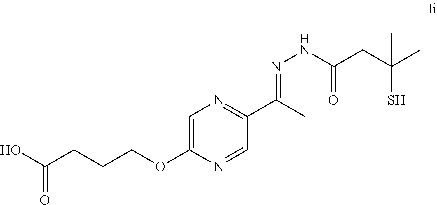 [SMILES: C/C(=N\NC(=O)CC(C)(C)S)c1cnc(OCCCC(=O)O)cn1]

Compound Ii was prepared using a similar procedure to compound Ia. It was collected as a yellow residue (141 mg, 92% yield, mixture of isomers). ¹H NMR (500 MHz, CHLOROFORM-d) δ ppm 9.70 (s, 1H), 8.74 (d, J=1.35 Hz, 1H), 8.03 (d, J=1.34 Hz, 1H), 4.41-4.44 (m, 2H), 3.11 (s, 2H), 2.48 (br d, J=6.36 Hz, 3H), 2.12 (br d, J=6.72 Hz, 2H), 1.94 (s, 3H), 1.43 (s, 6H); LCMS [M+H]⁺ 355.

### 4-((5-(2-(3-Mercapto-3-methylbutanoyl)hydrazineylidene)-5,6,7,8-tetrahydroquinoxalin-2-yl)oxy)butanoic acid (Ij)

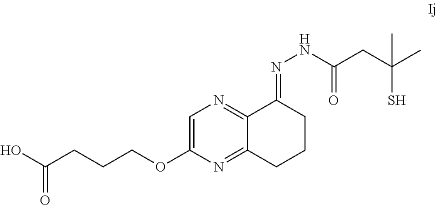 [SMILES: CC(C)(S)CC(=O)N/N=C1\CCCc2nc(OCCCC(=O)O)cnc21]

Compound Ij was prepared using a similar procedure to compound Ia. This reactions gave two fractions of componf Ij as mixtures of isomers. First fraction, light purple powder (96.7 mg, 31.8% yield). ¹H NMR (DMSO-d6, 500 MHz) δ 11.9-12.4 (m, 1H), 10.46 (s, 1H), 10.30 (s, 1H), 8.25 (s, 1H), 8.23 (s, 1H), 8.19 (s, 1H), 4.3-4.4 (m, 4H), 3.0-3.1 (m, 3H), 2.8-2.9 (m, 3H), 2.6-2.7 (m, 5H), 2.4-2.4 (m, 4H), 2.0-2.0 (m, 4H), 1.9-1.9 (m, 3H), 1.48 (s, 3H), 1.47 (s, 3H); LCMS [M+H]⁺ 381.2. Second fraction, dark purple foam (129.2 mg, 42.5% yield). LCMS [M+H]⁺ 381.4

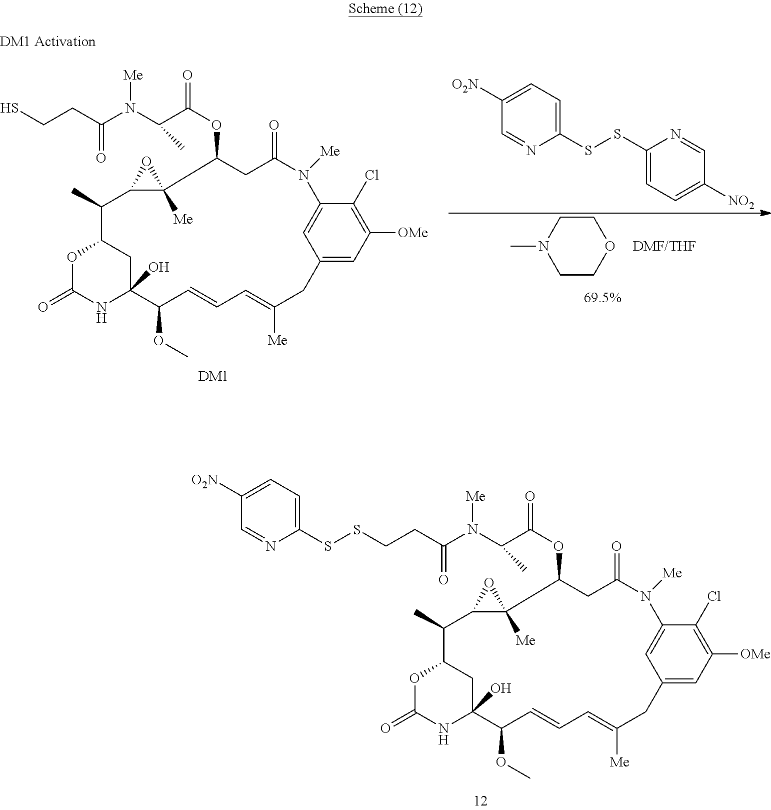

### DM1-thio(5-nitropyridine) (12)

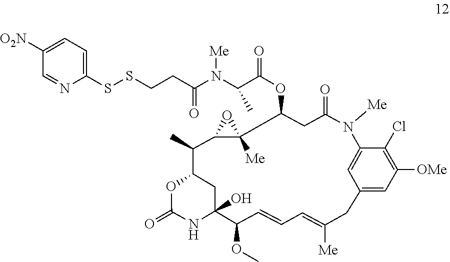

To a solution of 1,2-bis(5-nitropyridin-2-yl)disulfane (147 mg, 0.474 mmol) in THE (15 ml) was added 4-methylmorpholine (0.033 ml, 0.296 mmol). The mixture was stirred at room temperature upon which it was added to a solution of DM1 (175 mg, 0.237 mmol) in DMF (7.50 ml). The reaction mixture was stirred at room temperature for 90 min. LCMS showed that the reaction went to almost completion. Most of the THF was evaporated under reduced pressure. The resulting crude concentrate was diluted with EtOAc. The organic layer was washed with water (×3) then with brine. It was dried over Na₂SO₄ then concentrated. The crude product was purified using CombiFlash RF (12 g silica column: eluent 0—100% then 100% EtOAc/Hexanes) to afford the title compound 12 as a light yellow powder (147 mg, 69.5% yield). ¹H NMR (500 MHz, DMSO-d6) δ=9.11-9.09 (m, 1H), 8.45 (dd, J=2.4, 8.9 Hz, 1H), 7.88 (d, J=8.9 Hz, 1H), 7.00 (s, 1H), 6.88 (s, 1H), 6.58-6.49 (m, 2H), 6.37-6.35 (m, 1H), 5.93 (s, 1H), 5.57 (br dd, J=9.2, 13.7 Hz, 1H), 5.30 (q, J=6.7 Hz, 1H), 4.52 (br dd, J=2.0, 12.0 Hz, 1H), 4.09-4.00 (m, 2H), 3.87 (s, 3H), 3.48 (br d, J=8.9 Hz, 1H), 3.25 (s, 3H), 3.19-3.13 (m, 1H), 3.08 (s, 3H), 3.08-3.03 (m, 1H), 3.02-2.89 (m, 2H), 2.78 (br d, J=9.5 Hz, 1H), 2.70 (s, 3H), 2.46-2.39 (m, 1H), 2.03-1.97 (m, 2H), 1.54 (s, 3H), 1.49-1.41 (m, 2H), 1.24 (br d, J=13.0 Hz, 1H), 1.17 (br d, J=6.8 Hz, 3H), 1.12 (br d, J=6.2 Hz, 3H), 0.75 (s, 3H); LCMS [M+H]⁺ 893.

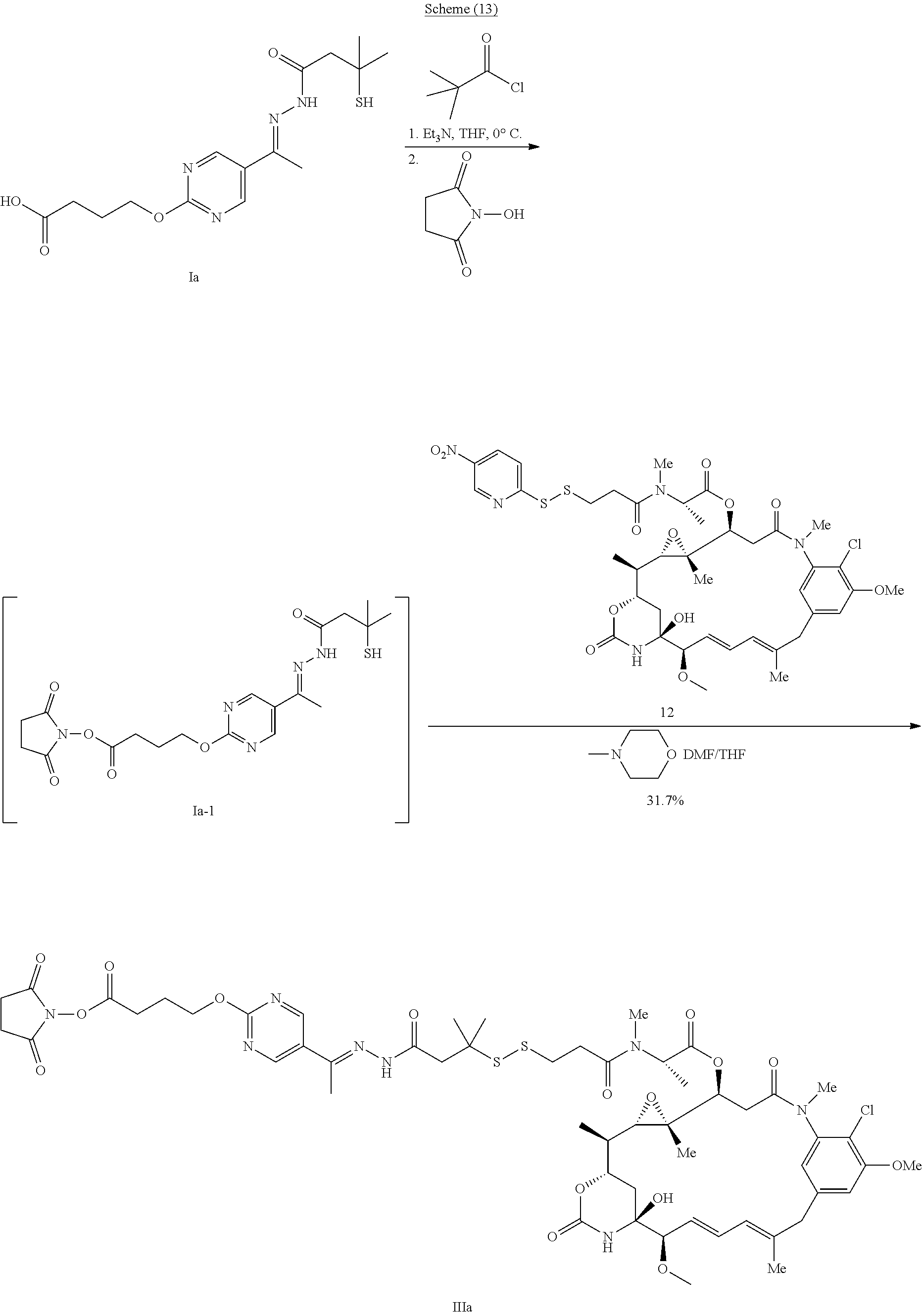

### Heterocyclic Linker-DM1 Construct (IIIa)

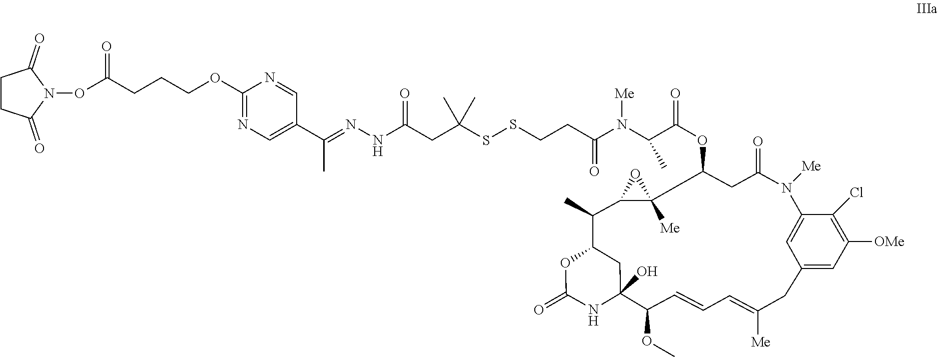

A 30 ml vial was charged with 4-((5-(1-(2-(3-mercapto-3-methylbutanoyl)hydrazono)ethyl)pyrimidin-2-yl)oxy)butanoic acid Ia (30 mg, 0.085 mmol) then THF (2 mL) was added. The solution was stirred at 0° C. upon which triethylamine (0.035 mL, 0.254 mmol) followed by trimethylacetyl chloride (0.011 mL, 0.093 mmol) were added. After stirring for 10 min, N-hydroxysuccinimide (10.72 mg, 0.093 mmol) was added. The reaction mixture was stirred for an additional 10 min then stopped. The Et₃N.HCl that has formed was filtered off and washed with THF to afford a solution of intermediate Ia-1 (2.8 mL). DM1-thio(5-nitropyridine) 12 (19.4 mg, 0.022 mol) was dissolved in DMF (1 mL) then Ia-1 in the filtrate (2.8 mL) was added. 4-Methylmorpholine (0.065 mL, 0.033 mmol) as a 0.5 M solution in DMF was added. The mixture was stirred at room temperature for 10 min upon which LCMS showed completion. The crude mixture was separated between water and EtOAc and shaken. The organic layer was washed with water (×3) then with brine. It was dried over Na₂SO₄ and concentrated down. The crude product was purified using CombiFlash RF (4 g Gold silica column eluent: EtOAc/Hexanes; 0-100% then 100% EtOAc). The product was taken into acetonitrile frozen then lyophilized. The title compound IIIa was collected as a white fluffy powder (11.7 mg, 31.7% yield, 2 isomers). ¹H NMR (DMSO-d6, 500 MHz) δ 10.55 (s, 1H), 10.36 (s, 1H), 8.88 (s, 1H), 8.85 (s, 1H), 7.09 (s, 1H), 7.04 (s, 1H), 6.80 (br d, 1H, J=5.6 Hz), 6.4-6.6 (m, 3H), 5.85 (br d, 1H, J=4.3 Hz), 5.45 (dt, 1H, J=9.6, 13.6 Hz), 5.2-5.3 (m, 1H), 4.4-4.5 (m, 1H), 4.34 (br t, 2H, J=5.7 Hz), 4.00 (br t, 1H, J=10.6 Hz), 3.84 (br d, 3H, J=4.8 Hz), 3.4-3.5 (m, 2H), 3.18 (br d, 3H, J=2.7 Hz), 3.09 (s, 1H), 3.05 (s, 2H), 2.8-2.9 (m, 1H), 2.8-2.8 (m, 3H), 2.75 (br s, 4H), 2.72 (br s, 1H), 2.65 (s, 1H), 2.62 (s, 2H), 2.57 (br s, 2H), 2.29 (br s, 2H), 2.05 (quin, 2H, J=6.7 Hz), 1.9-2.0 (m, 1H), 1.52 (s, 1H), 1.49 (s, 2H), 1.3-1.4 (m, 3H), 1.17 (br s, 3H), 1.15 (br s, 3H), 1.09 (br d, 3H, J=7.0 Hz), 1.05 (br d, 3H, J=4.5 Hz), 0.71 (br d, 3H, J=6.7 Hz); LCMS [M+H]⁺ 1187.

### Heterocyclic Linker-DM1 Construct (IIIb)

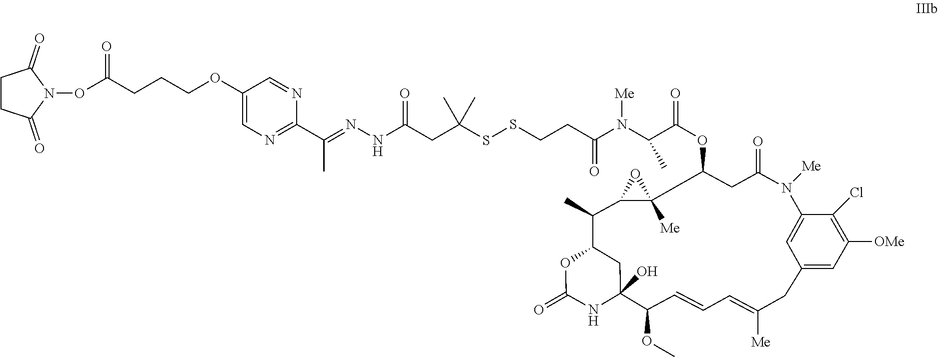

Heterocyclic linker-DM1 construct IIIb was prepared according to a similar procedure to heterocyclic linker-DM1 IIIa. It was collected as a white fluffy powder (17.2 mg, 73.3% yield, 2 isomers). ¹H NMR (DMSO-d6, 500 MHz) δ 10.63 (s, 1H), 10.42 (s, 1H), 8.67 (s, 1H), 8.66 (br s, 1H), 7.22 (s, 1H), 7.16 (s, 1H), 6.94 (br d, 1H, J=3.7 Hz), 6.6-6.7 (m, 3H), 5.98 (br d, 1H, J=7.8 Hz), 5.5-5.6 (m, 1H), 5.3-5.4 (m, 1H), 4.5-4.6 (m, 1H), 4.33 (q, 2H, J=6.3 Hz), 4.13 (br t, 1H, J=11.3 Hz), 3.98 (s, 1H), 3.96 (s, 1H), 3.54 (br dd, 2H, J=6.7, 8.7 Hz), 3.31 (s, 3H), 3.22 (s, 1H), 3.20 (s, 1H), 2.9-3.0 (m, 2H), 2.88 (br s, 4H), 2.78 (s, 1H), 2.74 (s, 1H), 2.65 (s, 1H), 2.39 (br d, 3H, J=8.6 Hz), 2.2-2.2 (m, 2H), 1.64 (s, 1H), 1.61 (s, 2H), 1.5-1.6 (m, 2H), 1.36 (s, 1H), 1.35 (s, 1H), 1.30 (br s, 6H), 1.22 (br t, 3H, J=7.3 Hz), 1.18 (br d, 3H, J=5.6 Hz), 0.84 (br d, 3H, J=6.5 Hz), LCMS [M+H]⁺ 1187.

### Heterocyclic Linker-DM1 Construct (IIIc)

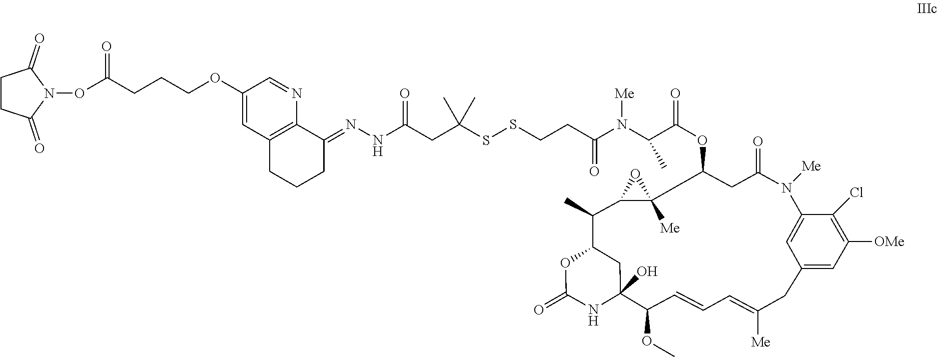

Heterocyclic linker-DM1 construct IIIc was prepared according to a similar procedure to heterocyclic linker-DM1 IIIa. It was collected as an off-white fluffy powder (15.6 mg, 52.8% yield, mixture of isomers). ¹H NMR (DMSO-d6, 500 MHz) δ 8.42 (d, 1H, J=2.8 Hz), 8.39 (d, 1H, J=2.8 Hz), 7.5-7.6 (m, 1H), 7.19 (br s, 1H), 6.94 (s, 1H), 6.7-6.7 (m, 1H), 6.6-6.6 (m, 2H), 6.0-6.0 (m, 1H), 5.5-5.6 (m, 1H), 5.37 (q, 1H, J=6.8 Hz), 4.57 (br dd, 1H, J=2.6, 12.0 Hz), 4.28 (br t, 2H, J=6.1 Hz), 4.2-4.2 (m, 1H), 4.13 (br t, 1H, J=11.2 Hz), 3.98 (br s, 1H), 3.95 (s, 1H), 3.94 (s, 1H), 3.5-3.6 (m, 2H), 3.31 (s, 2H), 3.30 (br s, 1H), 3.21 (s, 2H), 3.20 (s, 1H), 2.9-3.0 (m, 6H), 2.88 (br s, 4H), 2.7-2.8 (m, 4H), 2.7-2.7 (m, 3H), 2.4-2.5 (m, 2H), 2.2-2.2 (m, 2H), 2.1-2.1 (m, 1H), 1.95 (br d, 2H, J=4.8 Hz), 1.65 (br s, 1H), 1.63 (s, 2H), 1.61 (br d, 1H, J=4.0 Hz), 1.5-1.6 (m, 3H), 1.35 (br d, 1H, J=6.2 Hz), 1.32 (br s, 1H), 1.29 (br s, 3H), 1.29 (br s, 3H), 1.28 (br s, 1H), 1.26 (br s, 2H), 1.23 (br s, 3H), 1.22 (br d, 3H, J=2.9 Hz), 1.2-1.2 (m, 5H), 0.84 (s, 3H); LCMS [M+H]⁺ 1212.

### Heterocyclic Linker-DM1 Construct (IIId)

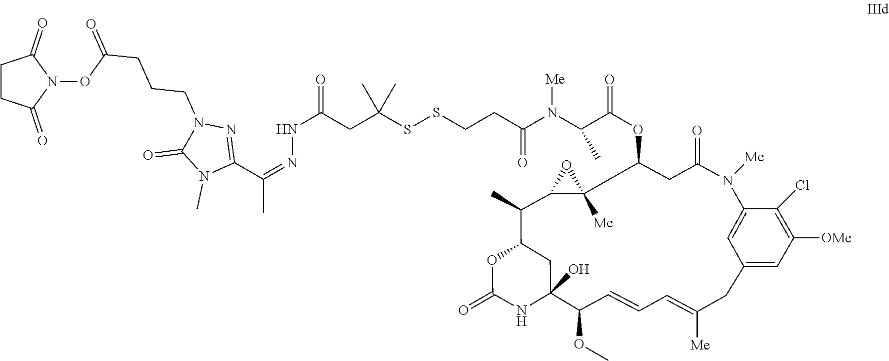

Heterocyclic linker-DM1 construct IIId was prepared according to a similar procedure to heterocyclic linker-DM1 IIIa. It was collected as a white fluffy powder (15.4 mg, 35.8% yield, 2 isomers). ¹H NMR (DMSO-d6, 500 MHz) δ 10.67 (s, 1H), 10.55 (s, 1H), 7.08 (br d, 1H, J=7.6 Hz), 6.81 (br d, 1H, J=4.4 Hz), 6.46 (br d, 3H, J=7.2 Hz), 5.85 (s, 1H), 5.46 (br dd, 1H, J=9.0, 14.9 Hz), 5.2-5.3 (m, 1H), 4.44 (br d, 1H, J=12.0 Hz), 4.00 (br t, 1H, J=11.4 Hz), 3.83 (s, 3H), 3.78 (t, 2H, J=6.9 Hz), 3.4-3.4 (m, 3H), 3.35 (s, 2H), 3.18 (s, 3H), 3.09 (s, 2H), 3.07 (5, 2H), 2.76 (br s, 2H), 2.74 (s, 4H), 2.71 (br d, 3H, J=7.2 Hz), 2.64 (d, 3H, J=6.5 Hz), 2.12 (s, 3H), 1.9-2.0 (m, 3H), 1.51 (br d, 3H, J=5.0 Hz), 1.3-1.4 (m, 3H), 1.22 (d, 1H, J=5.6 Hz), 1.18 (br s, 3H), 1.17 (br s, 3H), 1.10 (br dd, 3H, J=2.6, 6.7 Hz), 1.1-1.1 (m, 1H), 1.05 (br d, 3H, J=6.4 Hz), 0.71 (br d, 3H, J=3.3 Hz); LCMS [M+H]⁺ 1190.

### Heterocyclic Linker-DM1 Construct (IIIe)

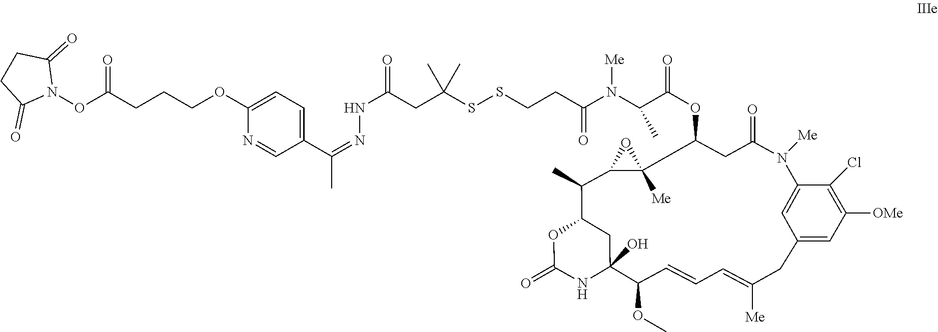

Heterocyclic linker-DM1 construct IIIe was prepared according to a similar procedure to heterocyclic linker-DM1 IIIa. It was collected as a white fluffy powder (3.2 mg, 16% yield, 2 isomers). ¹H NMR H NMR (500 MHz, DMSO-d6) δ ppm 10.25-10.46 (m, 1H), 8.43 (br d, J=9.17 Hz, 1H), 8.00-8.15 (m, 1H), 7.01-7.12 (m, 1H), 6.73-6.88 (m, 2H), 6.40-6.64 (m, 3H), 5.85 (br d, J=5.99 Hz, 1H), 5.46 (dt, J=14.52, 9.80 Hz, 1H), 5.24 (br t, J=6.79 Hz, 1H), 4.44 (br d, J=11.49 Hz, 1H), 4.29 (br s, 2H), 4.00 (br t, J=11.00 Hz, 1H), 3.83 (br d, J=5.01 Hz, 3H), 3.34-3.45 (m, 3H), 2.98-3.14 (m, 4H), 2.83-2.95 (m, 2H), 2.70-2.82 (m, 8H), 2.63 (br d, J=14.67 Hz, 3H), 2.17 (br d, J=6.11 Hz, 3H), 1.94-2.08 (m, 3H), 1.50 (br d, J=14.43 Hz, 3H), 1.32-1.44 (m, 3H), 1.16 (br d, J=8.68 Hz, 9H), 1.03-1.12 (m, 7H), 0.71 (br d, J=5.14 Hz, 3H); LCMS [M+H]⁺ 1186.

### Heterocyclic Linker-DM1 Construct (IIIf)

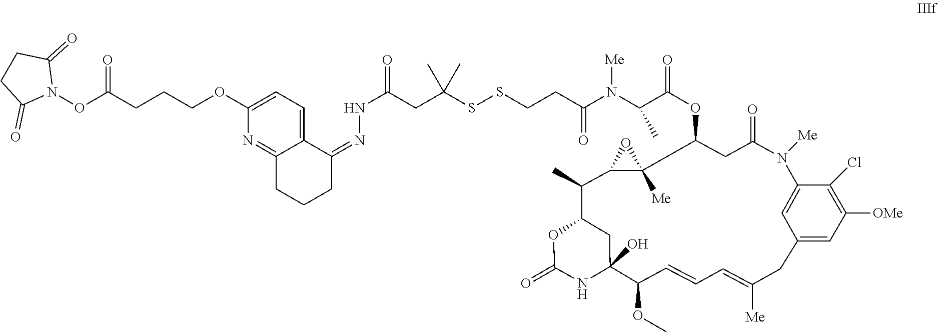

Heterocyclic linker-DM1 construct IIIf was prepared according to a similar procedure to heterocyclic linker-DM1 IIIa. It was collected as a white fluffy powder (3.1 mg, 11% yield, 2 isomers). ¹H NMR (500 MHz, DMSO-d6) δ ppm 10.19-10.43 (m, 1H), 8.11-8.23 (m, 1H), 7.01-7.11 (m, 1H), 6.82 (br d, J=6.24 Hz, 1H), 6.68 (br dd, J=15.10, 8.62 Hz, 1H), 6.43-6.60 (m, 3H), 5.86 (br d, J=6.48 Hz, 1H), 5.46 (dt, J=14.79, 9.35 Hz, 1H), 5.24 (br t, J=6.17 Hz, 1 H), 4.44 (br d, J=11.37 Hz, 1H), 4.26 (br d, J=4.03 Hz, 2H), 4.00 (br t, J=11.07 Hz, 1H), 3.84 (br d, J=6.24 Hz, 3H), 3.33-3.46 (m, 2H), 3.18 (br d, J=7.21 Hz, 3H), 3.07 (br d, J=15.04 Hz, 3H), 2.97-3.05 (m, 1H), 2.87-2.93 (m, 1H), 2.68-2.82 (m, 11H), 2.64 (br d, J=11.62 Hz, 3H), 2.53 (br s, 3H), 2.28-2.38 (m, 1 H), 1.98-2.04 (m, 2H), 1.80 (br d, J=5.26 Hz, 2H), 1.50 (br d, J=13.94 Hz, 3 H), 1.37 (br d, J=8.44 Hz, 3H), 1.16 (br s, 6H), 1.14 (br s, 1H), 1.10 (br d, J=5.62 Hz, 3H), 1.05 (br d, J=5.50 Hz, 3H), 0.71 (br d, J=4.40 Hz, 3H); LCMS [M+H]⁺ 1212.

### Heterocyclic linker-DM1 construct (IIIg)

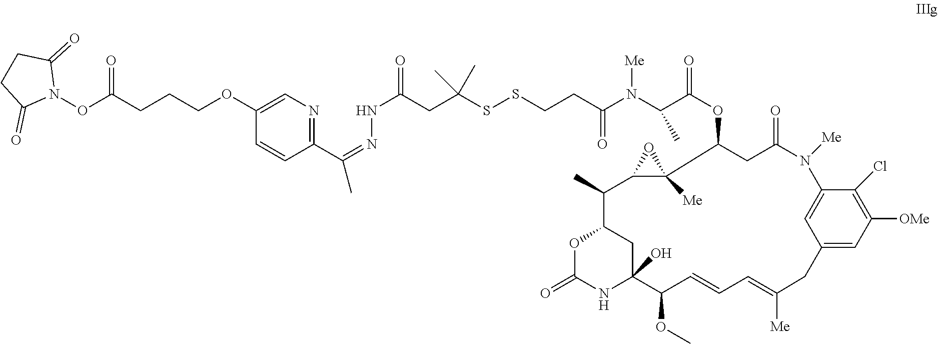 [SMILES: COc1cc2cc(c1Cl)N(C)C(=O)C[C@H](OC(=O)[C@H](C)N(C)C(=O)CCSSC(C)(C)CC(=O)N/N=C(/C)c1ccc(OCCCC(=O)ON3C(=O)CCC3=O)cn1)[C@]1(C)O[C@H]1[C@H](C)[C@@H]1C[C@@](O)(NC(=O)O1)[C@H](OC)/C=C/C=C(\C)C2]

Heterocyclic linker-DM1 construct IIIg was prepared according to a similar procedure to heterocyclic linker-DM1 IIIa. It was collected as a white fluffy powder (6.4 mg, 32% yield, 2 isomers). ¹H NMR (500 MHz, DMSO-d6) δ ppm 10.23-10.52 (m, 1H), 8.19-8.27 (m, 1H), 7.88-8.02 (m, 1H), 7.42 (ddd, J=18.83, 8.93, 2.69 Hz, 1H), 6.99-7.11 (m, 1H), 6.81 (br d, J=4.40 Hz, 1H), 6.41-6.60 (m, 3H), 5.85 (br d, J=8.31 Hz, 1H), 5.45 (td, J=14.70, 9.11 Hz, 1 H), 5.24 (quin, J=6.72 Hz, 1H), 4.40-4.50 (m, 1H), 4.07-4.14 (m, 1H), 4.00 (br t, J=11.25 Hz, 1H), 3.83 (br d, J=5.75 Hz, 3H), 3.32-3.45 (m, 2H), 3.17 (br d, J=10.64 Hz, 3H), 3.07 (br d, J=15.65 Hz, 3H), 3.00 (br d, J=12.10 Hz, 1H), 2.87-2.96 (m, 2H), 2.73-2.85 (m, 9H), 2.61-2.66 (m, 3H), 2.47-2.55 (m, 2 H), 2.23 (br d, J=7.70 Hz, 3H), 2.04 (quin, J=6.63 Hz, 2H), 1.97 (br d, J=14.67 Hz, 1H), 1.47-1.54 (m, 3H), 1.33-1.43 (m, 3H), 1.13-1.19 (m, 7H), 1.08-1.13 (m, 3H), 1.05 (br d, J=5.26 Hz, 3H), 0.71 (br d, J=5.50 Hz, 3H); LCMS [M+H]⁺ 1187.

### Heterocyclic Linker-DM1 Construct (IIIh)

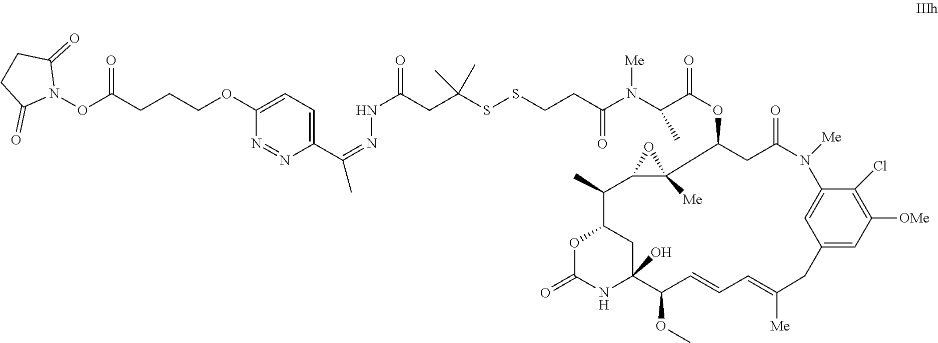 [SMILES: COc1cc2cc(c1Cl)N(C)C(=O)C[C@H](OC(=O)[C@H](C)N(C)C(=O)CCSSC(C)(C)CC(=O)N/N=C(/C)c1ccc(OCCCC(=O)ON3C(=O)CCC3=O)nn1)[C@]1(C)O[C@H]1[C@H](C)[C@@H]1C[C@@](O)(NC(=O)O1)[C@H](OC)/C=C/C=C(\C)C2]

Heterocyclic linker-DM1 construct IIIh was prepared according to a similar procedure to heterocyclic linker-DM1 IIIa. It was collected as a white fluffy powder (17 mg, 81% yield, 2 isomers). ¹H NMR (500 MHz, DMSO-d6) δ ppm 10.54-10.80 (m, 1H), 8.10-8.19 (m, 1H), 7.24-7.32 (m, 1H), 7.07-7.16 (m, 1H), 6.88 (br d, J=5.50 Hz, 1H), 6.49-6.65 (m, 3H), 5.92 (d, J=10.15 Hz, 1H), 5.44-5.60 (m, 1H), 5.25-5.36 (m, 1H), 4.49-4.57 (m, 3H), 4.07 (br t, J=11.13 Hz, 1H), 3.90 (d, J=4.52 Hz, 3H), 3.46-3.50 (m, 1H), 3.24 (d, J=8.68 Hz, 3H), 3.14 (d, J=15.16 Hz, 3H), 2.86-2.91 (m, 3H), 2.78-2.86 (m, 8H), 2.71 (br d, J=11.86 Hz, 3H), 2.40 (d, J=6.85 Hz, 3H), 2.13-2.19 (m, 2H), 2.01-2.07 (m, 1H), 1.56 (br d, J=17.36 Hz, 3H), 1.41-1.49 (m, 3H), 1.24 (br d, J=4.77 Hz, 6 H), 1.21 (s, 3H), 1.17 (br t, J=6.30 Hz, 4H), 1.10-1.14 (m, 4H), 0.78 (br d, J=6.97 Hz, 3H); LCMS [M+H]⁺ 1188.

### Heterocyclic Linker-DM1 Construct (IIIi)

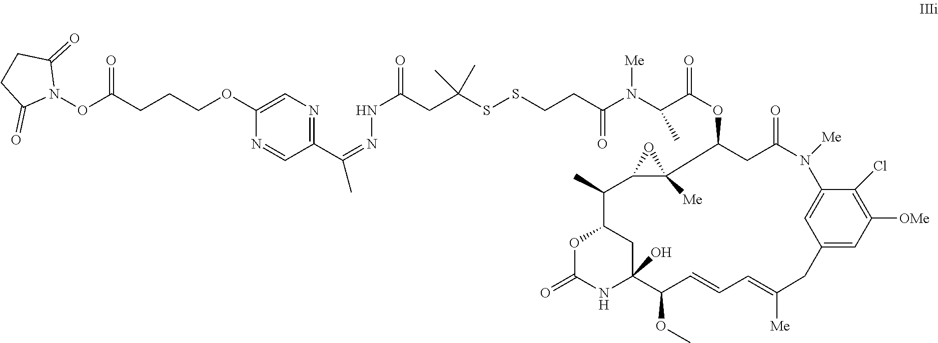 [SMILES: COc1cc2cc(c1Cl)N(C)C(=O)C[C@H](OC(=O)[C@H](C)N(C)C(=O)CCSSC(C)(C)CC(=O)N/N=C(/C)c1cnc(OCCCC(=O)ON3C(=O)CCC3=O)cn1)[C@]1(C)O[C@H]1[C@H](C)[C@@H]1C[C@@](O)(NC(=O)O1)[C@H](OC)/C=C/C=C(\C)C2]

Heterocyclic linker-DM1 construct IIIi was prepared according to a similar procedure to heterocyclic linker-DM1 IIIa. It was collected as a white fluffy powder (4 mg, 21% yield, 2 isomers). ¹H NMR (500 MHz, DMSO-d6) δ ppm 10.44-10.69 (m, 1H), 8.69-8.84 (m, 1H), 8.31 (br d, J=8.93 Hz, 1H), 7.10-7.21 (m, 1H), 6.89 (br s, 1H), 6.50-6.68 (m, 3H), 5.93 (br d, J=5.26 Hz, 1H), 5.53 (td, J=14.52, 9.11 Hz, 1H), 5.25-5.36 (m, 1H), 4.47-4.57 (m, 1H), 4.42 (br s, 2H), 4.07 (br t, J=10.39 Hz, 1H), 3.91 (br d, J=7.34 Hz, 3H), 3.40-3.54 (m, 2H), 3.25 (br s, 3H), 3.15 (br d, J=18.22 Hz, 3H), 2.95 (br d, J=14.06 Hz, 1 H), 2.80-2.91 (m, 10H), 2.71 (br d, J=13.33 Hz, 3H), 2.59 (br d, J=11.86 Hz, 1 H), 2.27-2.33 (m, 3H), 2.10-2.18 (m, 2H), 2.01-2.09 (m, 1H), 1.58 (br d, J=11.37 Hz, 3H), 1.45 (br s, 3H), 1.24 (br d, J=7.21 Hz, 8H), 1.11-1.20 (m, 6 H), 0.78 (br d, J=6.60 Hz, 3H); LCMS [M+H]⁺ 1187.

### Heterocyclic Linker-DM1 Construct (IIIj)

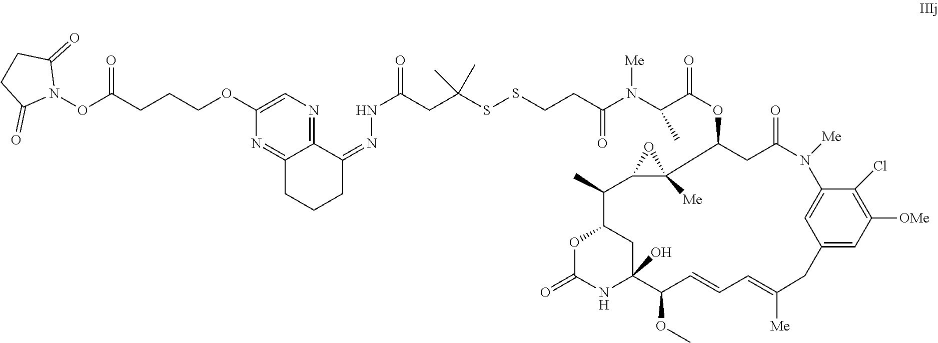 [SMILES: COc1cc2cc(c1Cl)N(C)C(=O)C[C@H](OC(=O)[C@H](C)N(C)C(=O)CCSSC(C)(C)CC(=O)N/N=C1/CCCc3nc(OCCCC(=O)ON4C(=O)CCC4=O)cnc31)[C@]1(C)O[C@H]1[C@H](C)[C@@H]1C[C@@](O)(NC(=O)O1)[C@H](OC)/C=C/C=C(\C)C2]

Heterocyclic linker-DM1 construct IIIj was prepared according to a similar procedure to heterocyclic linker-DM1 IIIa. It was collected as a light orange fluffy powder (7.86 mg, 28.3% yield). 1H NMR (DMSO-d6, 500 MHz) δ 13.71 (s, 1H), 13.28 (s, 1H), 8.3-8.4 (m, 1H), 8.3-8.3 (m, 1H), 7.1-7.2 (m, 1H), 6.88 (s, 1H), 6.6-6.7 (m, 1H), 6.5-6.6 (m, 2H), 5.9-5.9 (m, 1H), 5.53 (ddd, 1H, J=5.3, 9.0, 14.6 Hz), 5.32 (q, 1H, J=6.8 Hz), 4.51 (br d, 1H, J=12.0 Hz), 4.45 (br t, 2H, J=6.2 Hz), 4.4-4.4 (m, 1H), 4.08 (br t, 1H, J=11.5 Hz), 3.89 (s, 3H), 3.5-3.5 (m, 1H), 3.2-3.3 (m, 3H), 3.1-3.2 (m, 3H), 2.96 (br t, 3H, J=6.1 Hz), 2.9-2.9 (m, 4H), 2.83 (br s, 5H), 2.72 (s, 2H), 2.66 (br d, 2H, J=6.5 Hz), 2.55 (s, 6H), 2.4-2.4 (m, 2H), 2.1-2.2 (m, 2H), 2.06 (br s, 1H), 1.9-2.0 (m, 3H), 1.57 (s, 2H), 1.4-1.5 (m, 4H), 1.24 (br s, 3H), 1.2-1.2 (m, 3H), 1.2-1.2 (m, 5H), 1.14 (s, 2H), 1.12 (br s, 1H), 1.02 (s, 1H), 0.78 (s, 3H); LCMS [M+H]⁺ 1213.8.

Conjugation of DM1-linker construct of Formula (III) to Antibodies

In some embodiments, the linker-drug conjugate of Formula (III) is chemically conjugated to accessible lysine residues on antibodies. For example, as shown in Schemes 14 and 15, exemplary drug, cytotoxin microtubule inhibitor DM1, is chemically linked to surface accessible lysine residues on human IgG1 antibodies such as Cetuximab or Trastuzumab by reaction of linker-DM1 conjugates of Formula (III) with the respective antibody to provide the ADCs of Formula (IV).

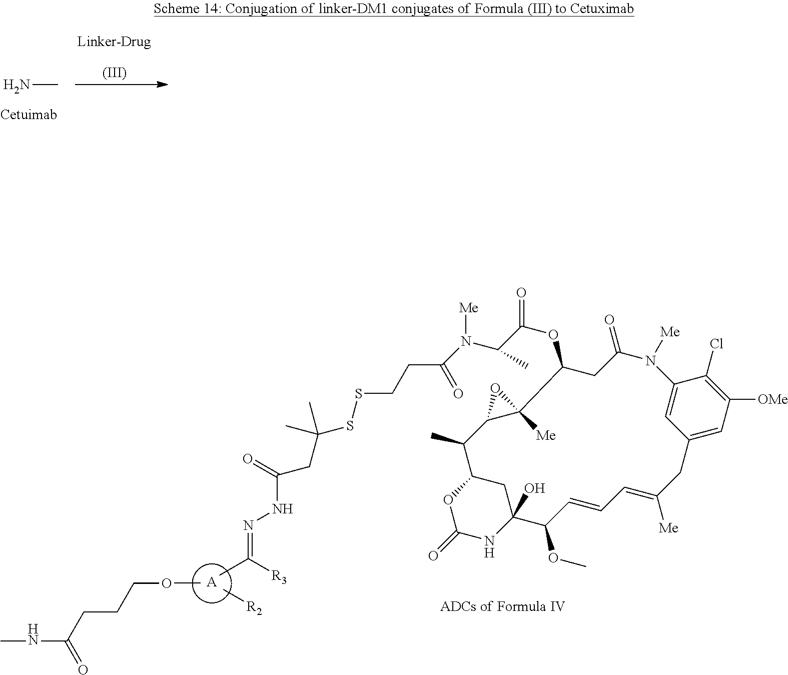

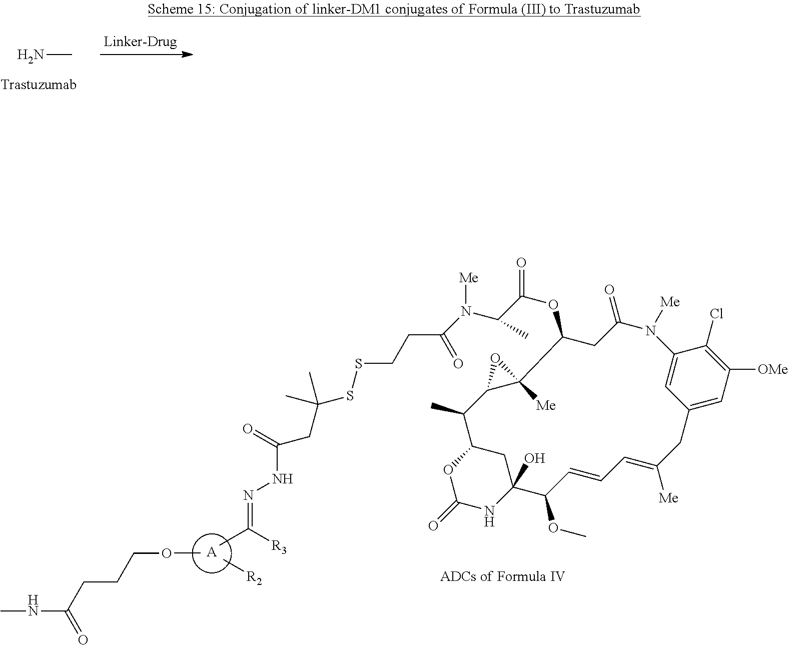

In an exemplary embodiment, cytotoxin microtubule inhibitor DM1 was chemically linked to surface accessible lysine residues on the human IgG1 antibody Trastuzumab by reaction of DM1-linker constructs (III) with the antibody.

Concentrated (10 mM) stock solutions of the linker with the attached DM1 payload of formula III were prepared in dimethylacetamide (DMA) and stored at −20° C. just prior to use. Prior to conjugation the concentrated stock was brought up to the temperature of 25° C. and then used to prepare a working stock in DMA equivalent to 5 times the desired concentration to be used in the reaction. The reaction mixture consisted of 13.3 μM of Trastuzumab, 66.5 μM Linker-DM1, 100 mM sodium phosphate, 20 mM NaCl, and pH 7.4. Once mixed, the reaction was incubated at 32° C. for 2.5 hours.

The reaction was stopped by buffer exchanging the sample into 20 mM sodium phosphate, 0.02% w/v Polysorbate 20 pH 7.4. Trehalose is then added to 6% w/v prior to storage at −80° C. Buffer exchange can be accomplished via gravity/spin desalting columns or tangential flow filtration methods.

Analysis of Bioconjugates

The absorbance of formulated bioconjugates was measured at 280 nm and one additional wavelength specific for the particular linker used. The extinction coefficient of this second wavelength was determined empirically for each combination of linker and payload used. The corresponding absorbance of the parental antibody was also measured at these two same wavelengths. The drug/antibody ratio was determined using the following equation. The second wavelength shown here is 252 nm, but this will depend on the particular linker-drug combination used,

$$DAR = \frac{( \frac{{A}_{252}}{{A}_{280} * {ε}_{Ab}^{280}} ) - {ε}_{A b}^{252}}{{ε}_{A D C}^{280} - ( \frac{{A}_{252}}{{A}_{280} * {ε}_{ADC}^{280}} )}$$

ADC—refers to the free linker-drug prior to conjugation

Ab—refers to the antibody prior to conjugation.

For conjugation with Trastuzumab a ratio of 10/1 (linker-drug/antibody) was used. Results are shown in Table 1 below. A positive control ADC (Trastuzumab-SMCC-DM1) and a negative control ADC (Synagis-SMCC-DM1) were also prepared.

TABLE 1
Conjugation with Trastuzumab

| ADC | Linker-Drug | DAR |
|---|---|---|
| Synagis-SMCC-DM1 | | 3.39 |
| IVa | IIIa | 3.28 |
| IVb | IIIb | 3.31 |
| IVd | IIId | 3.22 |
| IVf | IIIf | 4.16 |
| IVg | IIIg | 3.43 |
| IVh | IIIh | 3.31 |
| IVi | IIIi | 3.66 |
| Trastuzumab-SMCC- | | 3.56 |
| DM1 | | |

The conjugation reaction worked consistently with several drug linkers giving ADC s with yields>65% and a DAR ratio between 3.2 amd 4.2.

Biological Testing of Antibody-Drug Conjugates

The cytotoxic activity of Trastuzumab ADCs of Formula (IV) was tested against SKOV3 ovarian cell lines

SKOV3 ovarian cells were incubated with the effectors for a period corresponding to 2 to 3 times their estimated doubling time and the amount of viable cells was determine by measuring ATP content in the wells. ATP has been widely accepted as a valid marker of viable cells. When cells lose membrane integrity, they lose the ability to synthesize ATP and endogenous ATPases rapidly deplete any remaining ATP from the cytoplasm. All ADCs were diluted in DPBS to 6× the highest concentration tested, followed by 10 3-fold serial dilutions in DPBS for a total of 11 concentration points. Each point was added to triplicate wells. DPBS was added in wells to measure the maximum growth. Cells were diluted at their appropriate seeding density (ranging from 150 to 1000 cells per well) in complete media supplemented with glutamine 2 mM, serum and antibiotic cocktail. They were distributed in white, opaque bottom, tissue-culture treated 384 well plates and incubated for 24 hrs at 37° C.+5% CO₂. After addition of ADCs, cells were incubated at 37° C.+5% CO₂ for the appropriate amount of time (3 to 5 days) prior to cell viability count. Total ATP was measured using CellTiter-Glo™ reagent from Promega as recommended by the supplier. The cells and the reagent were equilibrated at RT for 30 min before mixing. Cell lysates were then incubated for 30 min to 1 hr at RT protected from light. Signal output was measured on a luminescence plate reader (Envision™, Perkin Elmer) set at an integration time of 0.1 sec. Integration time was adjusted to minimise signal saturation at high ATP concentration.

Data Analysis

Each concentration point (S) was normalized to the negative control wells (NC) and expressed as % survival (NC-S/NC×100). Potency (IC₅₀) and efficacy were calculated from a non-linear curve fit of the points versus log of the concentrations without constrain on the slope. Refined data were analysed using Prism™ software.

In this study the positive control ADC (Trastuzumab-SMCC-DM1) and negative control ADC (Synagis-SMCC-DM1) were used. The cytotoxicity data against SKOV3 ovarian cancer lines is shown in Table 2 below.

TABLE 2
Cytotoxicity of ADCs against SKOV3 cell lines

| ADC | IC₅₀ nM |
|---|---|
| Synagis-SMCC-DM1 | 4.01 |
| IVa | 0.992 |
| IVb | 0.893 |
| IVd | 1.14 |
| IVf | 0.487 |
| IVg | 1.07 |
| IVh | 1.08 |
| IVi | 1.43 |
| Trastuzumab-SMCC- | 0.587 |
| DM1 | |

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE APPLICATION

(1) a. In American Cancer Society. “Chemotherapy side effects” 2017. Available at https://www.cancer.org/treatment/treatments-and-side-effects/treatment-types/chemotherapy/chemotherapy-side-effects.html (Accessed Sep. 14, 2017);
   b. Beck, A.; Goetsch L.; Dumontet, C. and Corvaïa, N. Strategies and challenges of next generation antibody-drug conjugates, Nat. Rev. Drug. Discov. 2017, 16, 315-337.
(2) Chabner, B. A.; Roberts, T. G., Jr. Timeline: Chemotherapy and the war on cancer, Nat. Rev. Cancer 2005, 5, 65-72.
(3) Allen, T. M. Ligand-targeted therapeutics in anticancer therapy, Nat. Rev. Cancer 2002, 2, 750-763.
(4) Helleday, T.; Petermann, E.; Lundin, C.; Hodgson, B.; Sharma, R. A. DNA repair pathways as targets for cancer therapy, Nat. Rev. Cancer 2008, 8, 193-204.
(5) Zhang, J.; Yang, P. L.; Gray, N. S. Targeting cancer with small molecule kinase inhibitors, Nat. Rev. Cancer 2009, 9, 28-39.
(6) Aggarwal, S. Targeted cancer therapies, Nat. Rev. Drug. Discov. 2010, 9, 427-428.
(7) Tennant, D. A.; Duran, R. V.; Gottlieb, E. Targeting metabolic transformation for cancer therapy, Nat. Rev. Cancer 2010, 10, 267-277.
(8) Imai, K.; Takaoka, A. Comparing antibody and small-molecule therapies for cancer, Nat. Rev. Cancer 2006, 6, 714-727.
(9) Weiner, L. M.; Surana, R.; Wang, S. Monoclonal antibodies: versatile platforms for cancer immunotherapy, Nat. Rev. Immunol. 2010, 10, 317-327.
(10) Reichert, J. M. Antibody-based therapeutics to watch in 2011, MAbs 2011, 3, 76-99.
(11) Murphy, K. P.; Travers, P.; Walport, M.; Janeway, C. Janeway's Immunobiology; Garland Science: New York, 2008.
(12) Trail, P. A.; Willner, D.; Lasch, S. J.; Henderson, A. J.; Hofstead, S.; Casazza, A. M.; Firestone, R. A.; Hellstrom, I.; Hellstrom, K. E. Cure of xenografted human carcinomas by BR96-doxorubicin immunoconjugates, Science 1993, 261, 212-215.
(13) Alley, S. C.; Okeley, N. M.; Senter, P. D. Antibody-drug conjugates: targeted drug delivery for cancer, Curr. Opin. Chem. Biol. 2010, 14, 529-537.
(14) Ducry, L.; Stump, B. Antibody-drug conjugates: linking cytotoxic payloads to monoclonal antibodies, Bioconjug. Chem. 2010, 21, 5-13.
(15) Casi, G.; Neri, D. Antibody-drug conjugates: basic concepts, examples and future perspectives, J. Control. Release 2012, 161, 422-428.
(16) Adair, J. R.; Howard, P. W.; Hartley, J. A.; Williams, D. G.; Chester, K. A. Antibody-drug conjugates—a perfect synergy, Expert Opin. Biol. Ther. 2012, 12, 1191-1206.
(17) Carter, P. J. Potent antibody therapeutics by design, Nat. Rev. Immunol. 2006, 6, 343-357.
(18) Teicher, B. A. Antibody-drug conjugate targets, Curr. Cancer Drug Targets 2009, 9, 982-1004.
(19) Doronina, S. O.; Toki, B. E.; Torgov, M. Y.; Mendelsohn, B. A.; Cerveny, C. G.; Chace, D. F.; DeBlanc, R. L.; Gearing, R. P.; Bovee, T. D.; Siegall, C. B.; Francisco, J. A.; Wahl, A. F.; Meyer, D. L.; Senter, P. D. Development of potent monoclonal antibody auristatin conjugates for cancer therapy, Nat. Biotechnol. 2003, 21, 778-784.
(20) Widdison, W. C.; Wilhelm, S. D.; Cavanagh, E. E.; Whiteman, K. R.; Leece, B. A.; Kovtun, Y.; Goldmacher, V. S.; Xie, H.; Steeves, R. M.; Lutz, R. J.; Zhao, R.; Wang, L.; Blattler, W. A.; Chari, R. V. Semisynthetic maytansine analogues for the targeted treatment of cancer, J. Med. Chem. 2006, 49, 4392-4408.
(21) Hartley, J. A. The development of pyrrolobenzodiazepines as antitumour agents, Expert Opin. Investig. Drugs 2011, 20, 733-744.
(22) Doronina, S. O.; Mendelsohn, B. A.; Bovee, T. D.; Cerveny, C. G.; Alley, S. C.; Meyer, D. L.; Oflazoglu, E.; Toki, B. E.; Sanderson, R. J.; Zabinski, R. F.; Wahl, A. F.; Senter, P. D. Enhanced activity of monomethylauristatin F through monoclonal antibody delivery: effects of linker technology on efficacy and toxicity, Bioconjug. Chem. 2006, 17, 114-124.
(23) Dosio, F.; Brusa, P.; Cattel, L. Immunotoxins and Anticancer Drug Conjugate Assemblies: The Role of the Linkage between Components, Toxins (Basel) 2011, 3, 848-883.
(24) Wu, A. M.; Senter, P. D. Arming antibodies: prospects and challenges for immunoconjugates, Nat. Biotechnol. 2005, 23, 1137-1146.
(25) Ansell, S. M. Brentuximab vedotin: delivering an antimitotic drug to activated lymphoma cells, Expert Opin. Investig. Drugs 2011, 20, 99-105.
(26) Katz, J.; Janik, J. E.; Younes, A. Brentuximab Vedotin (SGN-35), Clin. Cancer Res. 2011, 17, 6428-6436.
(27) Hamann, P. R.; Hinman, L. M.; Hollander, I.; Beyer, C. F.; Lindh, D.; Holcomb, R.; Hallett, W.; Tsou, H. R.; Upeslacis, J.; Shochat, D.; Mountain, A.; Flowers, D. A.; Bernstein, I. Gemtuzumab ozogamicin, a potent and selective anti-CD33 antibody-calicheamicin conjugate for treatment of acute myeloid leukemia, Bioconjug. Chem. 2002, 13, 47-58.
(28) Hutter, M. L.; Schlenk, R. F. Gemtuzumab ozogamicin in non-acute promyelocytic acute myeloid leukemia, Expert Opin. Biol. Ther. 2011, 11, 1369-1380.
(29) Remillard, S.; Rebhun, L. I.; Howie, G. A.; Kupchan, S. M. Antimitotic activity of the potent tumor inhibitor maytansine, Science 1975, 189, 1002-1005.
(30) Goldberg, R. M. Cetuximab, Nat. Rev. Drug Discov. 2005, Suppl, S10-11.
(31) Kabolizadeh, P.; Kubicek, G. J.; Heron, D. E.; Ferris, R. L.; Gibson, M. K. The role of cetuximab in the management of head and neck cancers, Expert Opin. Biol. Ther. 2012, 12, 517-528.
(32) Broadbridge, V. T.; Karapetis, C. S.; Price, T. J. Cetuximab in metastatic colorectal cancer, Expert Rev. Anticancer Ther. 2012, 12, 555-565.
(33) Arteaga, C. L. Overview of epidermal growth factor receptor biology and its role as a therapeutic target in human neoplasia, Semin. Oncol. 2002, 29, 3-9.
(34) Mendelsohn, J.; Baselga, J. Status of epidermal growth factor receptor antagonists in the biology and treatment of cancer, J. Clin. Oncol. 2003, 21, 2787-2799.
(35) Krause, D. S.; Van Etten, R. A. Tyrosine kinases as targets for cancer therapy, N. Engl. J. Med. 2005, 353, 172-187.
(36) Goldstein, N. I.; Prewett, M.; Zuklys, K.; Rockwell, P.; Mendelsohn, J. Biological efficacy of a chimeric antibody to the epidermal growth factor receptor in a human tumor xenograft model, Clin. Cancer Res. 1995, 1, 1311-1318.
(37) Hamann, P. R.; Hinman, L. M.; Beyer, C. F.; Lindh, D.; Upeslacis, J.; Flowers, D. A.; Bernstein, I. An anti-CD33 antibody-calicheamicin conjugate for treatment of acute myeloid leukemia. Choice of linker, Bioconjug. Chem. 2002, 13, 40-46.
(38) van Der Velden, V. H.; te Marvelde, J. G.; Hoogeveen, P. G.; Bernstein, I. D.; Houtsmuller, A. B.; Berger, M. S.; van Dongen, J. J. Targeting of the CD33-calicheamicin immunoconjugate Mylotarg (CMA-676) in acute myeloid leukemia: in vivo and in vitro saturation and internalization by leukemic and normal myeloid cells, Blood 2001, 97, 3197-3204.
(39) Ojima, I.; Geng, X.; Wu, X.; Qu, C.; Borella, C. P.; Xie, H.; Wilhelm, S. D.; Leece, B. A.; Bartle, L. M.; Goldmacher, V. S.; Chari, R. V. Tumor-specific novel taxoid-monoclonal antibody conjugates, J. Med. Chem. 2002, 45, 5620-5623.
(40) Erickson, H. K.; Park, P. U.; Widdison, W. C.; Kovtun, Y. V.; Garrett, L. M.; Hoffman, K.; Lutz, R. J.; Goldmacher, V. S.; Blattler, W. A. Antibody-maytansinoid conjugates are activated in targeted cancer cells by lysosomal degradation and linker-dependent intracellular processing, Cancer Res. 2006, 66, 4426-4433.
(41) Lewis Phillips, G. D.; Li, G.; Dugger, D. L.; Crocker, L. M.; Parsons, K. L.; Mai, E.; Blattler, W. A.; Lambert, J. M.; Chari, R. V.; Lutz, R. J.; Wong, W. L.; Jacobson, F. S.; Koeppen, H.; Schwall, R. H.; Kenkare-Mitra, S. R.; Spencer, S. D.; Sliwkowski, M. X. Targeting HER2-positive breast cancer with trastuzumab-DM1, an antibody-cytotoxic drug conjugate, Cancer Res. 2008, 68, 9280-9290.
(42) Kellogg, B. A.; Garrett, L.; Kovtun, Y.; Lai, K. C.; Leece, B.; Miller, M.; Payne, G.; Steeves, R.; Whiteman, K. R.; Widdison, W.; Xie, H.; Singh, R.; Chari, R. V.; Lambert, J. M.; Lutz, R. J. Disulfide-linked antibody-maytansinoid conjugates: optimization of in vivo activity by varying the steric hindrance at carbon atoms adjacent to the disulfide linkage, Bioconjug. Chem. 2011, 22, 717-727.
(43) Dubowchik, G. M.; Mosure, K.; Knipe, J. O.; Firestone, R. A. Cathepsin B-sensitive dipeptide prodrugs. 2. Models of anticancer drugs paclitaxel (Taxol), mitomycin C and doxorubicin, Bioorg. Med. Chem. Lett. 1998, 8, 3347-3352.
(44) Francisco, J. A.; Cerveny, C. G.; Meyer, D. L.; Mixan, B. J.; Klussman, K.; Chace, D. F.; Rejniak, S. X.; Gordon, K. A.; DeBlanc, R.; Toki, B. E.; Law, C. L.; Doronina, S. O.; Siegall, C. B.; Senter, P. D.; Wahl, A. F. cAC10-vcMMAE, an anti-CD30-monomethyl auristatin E conjugate with potent and selective antitumor activity, Blood 2003, 102, 1458-1465.
(45) Sanderson, R. J.; Hering, M. A.; James, S. F.; Sun, M. M.; Doronina, S. O.; Siadak, A. W.; Senter, P. D.; Wahl, A. F. In vivo drug-linker stability of an anti-CD30 dipeptide-linked auristatin immunoconjugate, Clin. Cancer Res. 2005, 11, 843-852.
(46) DiJoseph, J. F.; Dougher, M. M.; Evans, D. Y.; Zhou, B. B.; Damle, N. K. Preclinical anti-tumor activity of antibody-targeted chemotherapy with CMC-544 (inotuzumab ozogamicin), a CD22-specific immunoconjugate of calicheamicin, compared with non-targeted combination chemotherapy with CVP or CHOP, Cancer Chemother. Pharmacol. 2011, 67, 741-749.
(47) Toki, B. E.; Cerveny, C. G.; Wahl, A. F.; Senter, P. D. Protease-mediated fragmentation of p-amidobenzyl ethers: a new strategy for the activation of anticancer prodrugs, J. Org. Chem. 2002, 67, 1866-1872.
(48) Kovtun, Y. V.; Audette, C. A.; Ye, Y.; Xie, H.; Ruberti, M. F.; Phinney, S. J.; Leece, B. A.; Chittenden, T.; Blattler, W. A.; Goldmacher, V. S. Antibody-drug conjugates designed to eradicate tumors with homogeneous and heterogeneous expression of the target antigen, Cancer Res. 2006, 66, 3214-3221.
(49) a. Dugger, R. B.; LeTendre, L. J.; Patel, V. B.; Prashad, A. S. and Zhang, C. Intermediates and methods for synthesizing Calicheamicin derivatives. WO 2015/063680 A1.
   b. Moran, J. K. and G, J. Processes for the convergent synthesis of Calicheamicin derivatives. US 2009/0312530 A1.
   c. Chiarello, G. A. and Sahli, A. Improved processes for making hydrazides. WO 2008/147765 A1.

1. A compound of Formula (I): 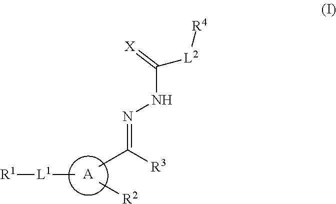
or a pharmaceutically acceptable salt and/or solvate thereof,
wherein:
Ring A is a 5 or 6 membered unsaturated heterocycloalkyl or 5 or 6 membered heteraromatic ring each comprising 1 to 4 heteroatoms selected from O, N and S, and Ring A is optionally substituted with one or more substituents independently selected from CN, NO₂, halo, C₁₋₆alkyl, C₁₋₆fluoroalkyl, ═O, OR⁵, SR⁵ and NR⁵R⁶;
R¹ and R⁴ are independently, a reactive functional group;
R² is absent or selected from H, CN, NO₂, halo, C₁₋₆alkyl, C₁₋₆fluoroalkyl, OR⁷, SR⁷ and NR⁷R⁸, and when present R² is ortho to 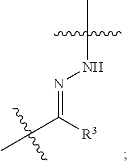
R³ is from H, C₁₋₄alkyl and C₁₋₄fluoroalkyl; or
R² and R³ are joined to form, together with the atoms therebetween, a 4 to 6 membered saturated or unsaturated ring, optionally containing one additional heteroatom selected from O, N and S, and optionally substituted with one or more substituents selected from C₁₋₆alkyl and C₁₋₆fluoroalkyl; and
X is selected from O, S and NR⁹;
R⁵, R⁶, R⁷, R⁸, and R⁹ are independently selected from H, C₁₋₆alkyl and C₁₋₆fluoroalkyl; and
L¹ and L² are independently a linker moiety.

2. The compound of claim 1, wherein L¹ and L² independently comprise at least one ester, carbonate, carbamate or amide linkage and optionally one or more ether, sulfone, sulfoxide, thioether, thioamide, thioester and amine, and optionally one or more C₁-C₂₀alkylene groups, C₂-C₂₀alkenylene groups and C₂-C₂₀alkynylene groups.

3. The compound of claim 1, wherein L¹ and L² are independently selected from a direct bond, Z, R^{a}, Z—R^{a}, R^{a}—Z, R^{a}—Z—R^{b} and Z—R^{a}—Z^{a}, wherein Z and Z^{a} are independently selected from O, S, S(O), SO₂, NH, N(C₁₋₆alkyl), C(Q), C(Q)Y, YC(Q), YC(Q)Y^{a}, (C₁₋₆alkyleneY)ₚ and Y—(C₁₋₆alkyleneY)ₚ, wherein R^{a} and R^{b} are independently selected from C₁₋₁₀alkylene, C₂₋₁₀alkenylene and C₂₋₁₀alkynylene; Q, Y and Y^{a} are independently selected from O, S, NH and N(C₁₋₆alkyl); and p is selected from 1, 2, 3, 4, 5 and 6.

4. The compound of claim 3, wherein R^{a} and R^{b} are independently selected from C₁₋₆alkylene, C₂₋₆alkenylene and C₂₋₆alkynylene.

5. The compound of claim 3 or 4, wherein Q, Y and Y^{a} are independently selected from O, S, NH and N(CH₃).

6. The compound of any one of claims 3 to 5, wherein Z and Z^{a} are independently selected from O, S, S(O), SO₂, NH, N(CH₃), C(O), C(O)NH, NHC(O), NHC(O)O, OC(O)O, NHC(O)NH, OC(O)NH, NHC(NH)NH, (C₁₋₆alkyleneO)ₚ and O—(C₁₋₆alkyleneO)ₚ.

7. The compound of claim 1, wherein L¹ is selected from OC(O)C₁₋₁₀alkyleneO, NHC(O)C₁₋₁₀alkyleneO, C₁₋₆alkyleneO, OC(O)C₁₋₁₀alkyleneNH, NHC(O)C₁₋₁₀alkyleneNH, C₁₋₆alkyleneNH, C(O)C₁₋₁₀alkyleneO and C(O)C₁₋₁₀alkyleneNH.

8. The compound of claim 1 or 7, wherein L² is selected from C₁₋₁₀alkyleneS and C₁₋₁₀alkylene.

9. The compound of any one of claims 1 to 8, wherein Ring A is a 5 or 6 membered heteroaromatic ring.

10. The compound of claim 9, wherein Ring A is selected from pyridinyl, pyrimidinyl, pyridazinyl, pyrazinyl, triazinyl, tetrazinyl, thienyl, furanyl, pyrrolyl, triazolyl, thiazolyl, oxazolyl and pyrazolyl.

11. The compound of any one of claims 1 to 8, wherein Ring A is selected from pyridinyl, pyrimidinyl, pyrazinyl and pyridazinyl.

12. The compound of any one of claims 1 to 8, wherein L¹ is located in the position para to 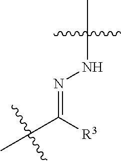
on Ring A.

13. The compound of any one of claims 1 to 12, wherein Ring A is optionally substituted with one or three substituents are independently selected from CN, halo, C₁₋₆alkyl and C₁₋₆fluoroalkyl.

14. The compound of claim 13, wherein Ring A is optionally substituted with one or two substituents are independently selected from CH₃, CF₃, CH₂CH₃, CH₂CH₂F, CH₂CF₂H and CH₂CF₃

15. The compound of any one of claims 1 to 14, wherein R² is absent.

16. The compound of any one of claims 1 to 14, wherein R² is selected from H, CN, NO₂, halo, C₁₋₆alkyl, C₁₋₆fluoroalkyl, OR⁷ and SR⁷.

17. The compound of claim 16, wherein R² is selected from H, CN, halo, C₁₋₆alkyl and C₁₋₆fluoroalkyl.

18. The compound of any one of claims 1 to 17, wherein R³ is selected from H, CH₃, CF₃, CH₂CH₃, CH₂CH₂F, CH₂CF₂H and CH₂CF₃.

19. The compound of any one of claims 1 to 14, wherein R² and R³ are joined to form, together with the atoms therebetween, a 5 to 6 membered saturated or unsaturated ring, optionally substituted with one or more substituents selected from C₁₋₆alkyl and C₁₋₆fluoroalkyl.

20. The compound of claim 19, wherein R² and R³ are joined to form a 6 membered saturated or unsaturated ring, optionally substituted with one or two substituents selected from C₁₋₆alkyl and C₁₋₆fluoroalkyl.

21. The compound of claim 20, wherein R² and R³ are joined to form a 6 membered unsaturated ring.

22. The compound of any one of claims 1 to 21, wherein R⁵, R⁶, R⁷, R⁸, and R⁹ are independently selected from H, C₁₋₄alkyl and C₁₋₄fluoroalkyl.

23. The compound of any one of claims 1 to 21, wherein R⁵ and R⁷ are independently selected from methyl, ethyl, propyl, isopropyl, sec-butyl, n-butyl and t-butyl.

24. The compound of any one of claims 1 to 21, wherein R⁶ and R⁸ are H.

25. The compound of any one of claims 1 to 24, wherein R¹ and R⁴ are independently selected from a nucleophilic group and an electrophilic group.

26. The compound of any one of claims 1 to 24, wherein R¹ and R⁴ are independently selected from Michael addition acceptors, olefins, acetylenes, alcohols, phenols, ethers, oxides, halides, aldehydes, ketones, carboxylic acids, esters, amines, thiols, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids, isonitriles, amidines, imides, imidates, nitrones, hydroxylamines, oximes, hydroxamic acids, thiohydroxamic acids, allenes, ortho esters, sulfites, enamines, ureas, semicarbazides, carbodiimides, carbamates, imines, azides, azo compounds and nitroso compounds.

27. The compound of any one of claims 1 to 24, wherein R¹ and R⁴ are independently selected from Michael addition acceptors, amines, maleimide, N-hydroxysuccinimide ester and thiols.

28. The compound of any one of claims 1 to 27, wherein X is O.

29. The compound of claim 1, wherein the compound has the following structure: 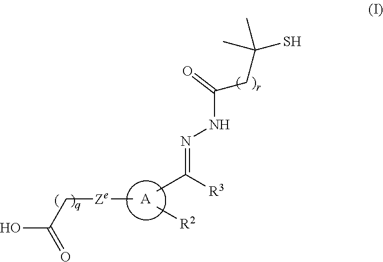
or a pharmaceutically acceptable salt and/or solvate thereof,
wherein
R² and R³ are as defined in any one of claims 15-21;
Z^{e} is selected from C(O)NH and O; and
q and r are independently 1, 2, 3, 4, 5, 6, 7 or 8.

30. The compound of claim 1, wherein the compound has the following structure: 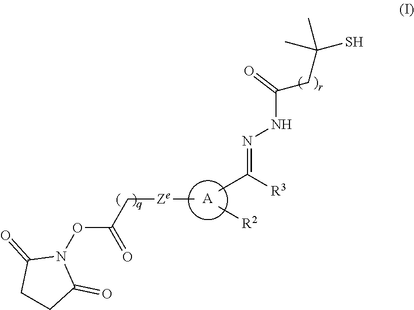
or a pharmaceutically acceptable salt and/or solvate thereof, wherein
R² and R³ are as in any one of claims 15-21;
Z^{e} is selected from C(O)NH and O; and
q and r are independently 1, 2, 3, 4, 5, 6, 7 or 8.

31. The compound of claim 1, wherein the compound is selected from; 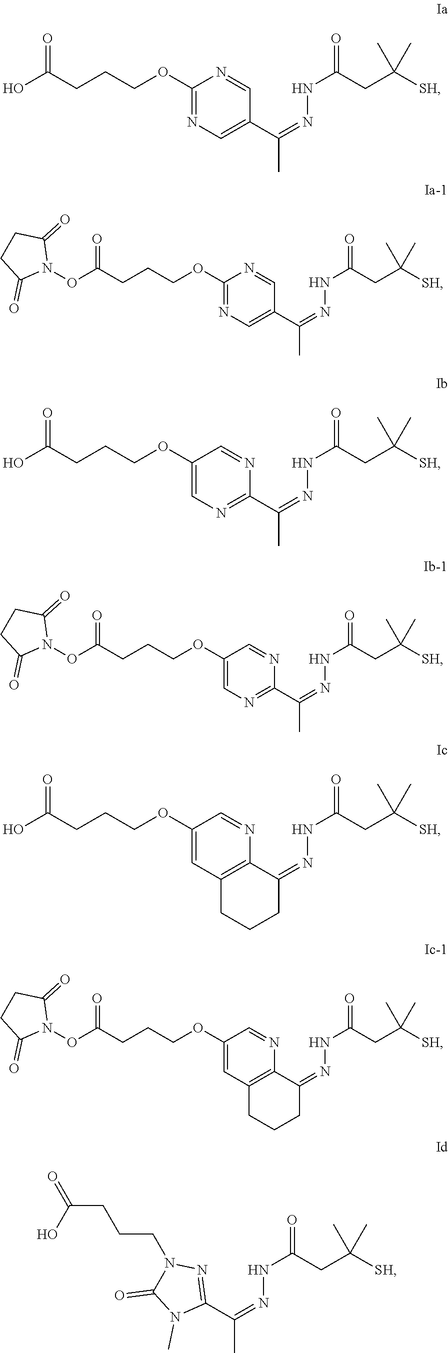 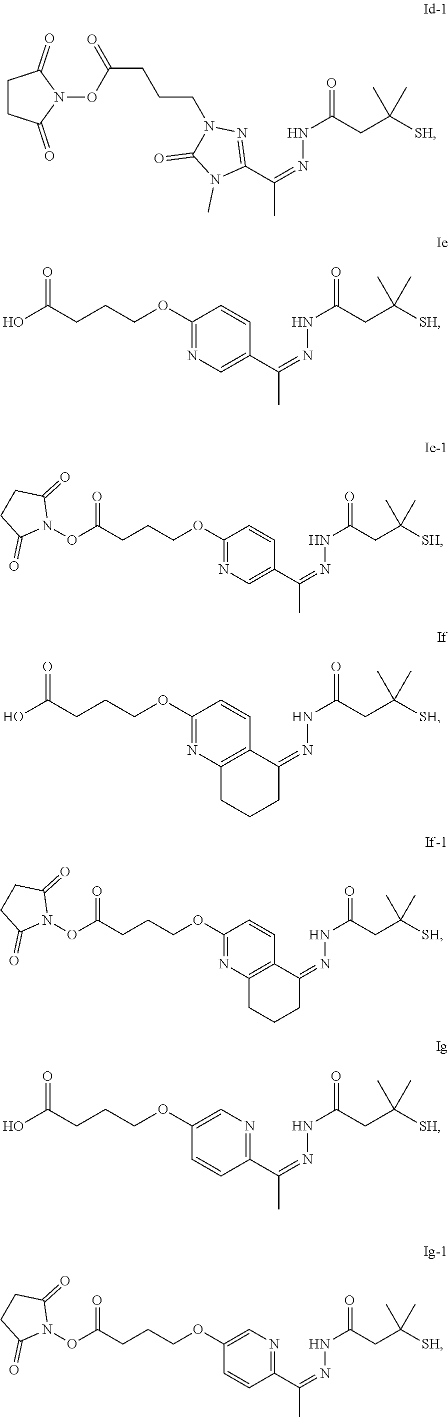 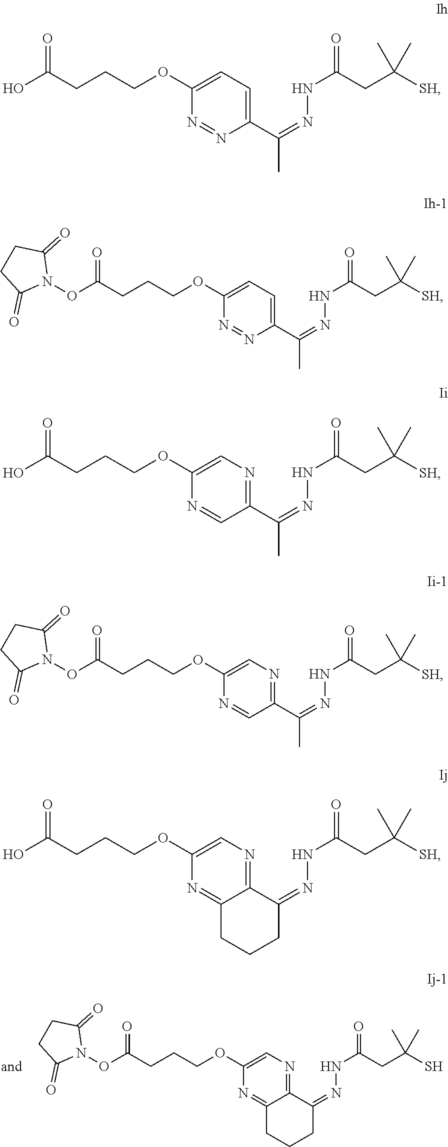 [SMILES: C/C(=N/NC(=O)CC(C)(C)S)c1ccc(OCCCC(=O)O)nn1 ; C/C(=N/NC(=O)CC(C)(C)S)c1ccc(OCCCC(=O)ON2C(=O)CCC2=O)nn1 ; C/C(=N/NC(=O)CC(C)(C)S)c1cnc(OCCCC(=O)O)cn1 ; C/C(=N/NC(=O)CC(C)(C)S)c1cnc(OCCCC(=O)ON2C(=O)CCC2=O)cn1 ; CC(C)(S)CC(=O)N/N=C1/CCCc2nc(OCCCC(=O)O)cnc21 ; CC(C)(S)CC(=O)N/N=C1/CCCc2nc(OCCCC(=O)ON3C(=O)CCC3=O)cnc21]
or a pharmaceutically acceptable salt and/or solvate thereof.

32. A compound of Formula (II) or a salt and/or solvate thereof: 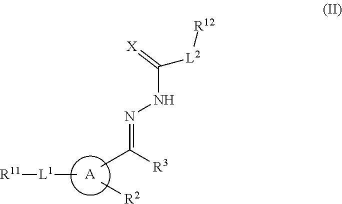
or a pharmaceutically acceptable salt and/or solvate thereof,
wherein:
Ring A is a 5 or 6 membered unsaturated heterocycloalkyl or 5 or 6 membered heteraromatic ring each comprising 1 to 4 heteroatoms selected from O, N and S, and Ring A is optionally substituted with one or more substituents independently selected from CN, NO₂, halo, C₁₋₆alkyl, C₁₋₆fluoroalkyl, ═O, OR⁵, SR⁵ and NR⁵R⁶;
R² is absent or selected from H, CN, NO₂, halo, C₁₋₆alkyl, C₁₋₆fluoroalkyl, OR⁷, SR⁷ and NR⁷R⁸, and when present R² is ortho to 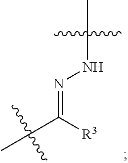
R³ is from H, C₁₋₄alkyl and C₁₋₄fluoroalkyl; or
R² and R³ are joined to form, together with the atoms therebetween, a 4 to 6 membered saturated or unsaturated ring, optionally containing one additional heteroatom selected from O, N and S, and optionally substituted with one or more substituents selected from C₁₋₆alkyl and C₁₋₆fluoroalkyl; and
X is selected from O, S and NR⁹;
R⁵, R⁶, R⁷, R⁸, and R⁹ are independently selected from H, C₁₋₆alkyl and C₁₋₆fluoroalkyl; and
L¹ and L² are independently a linker moiety; and
R¹¹ and R¹² are different and are selected from compounds to be linked together.

33. The compound of claim 32, wherein R¹¹ and R¹² are independently selected from a fluorescent dye, ligand, drug, small molecule, antibody, lipid, carbohydrate, nucleic acid, peptide, radiolabel, spin label, redox molecule, isotope label, PET label, nanoparticle, polymer, macrocycle, metal complex and solid support.

34. The compound claim 32 or 33, wherein R¹¹ and R¹² are independently selected from an antibody and drug.

35. An antibody-drug conjugate comprising an antibody covalently attached by a linker to one or more drugs, the conjugate having a Formula (IV): 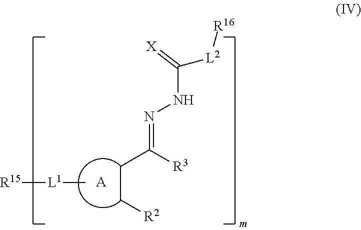
or a pharmaceutically acceptable salt and/or solvate thereof,
wherein
Ring A is a 5 or 6 membered unsaturated heterocycloalkyl or 5 or 6 membered heteraromatic ring each comprising 1 to 4 heteroatoms selected from O, N and S, and Ring A is optionally substituted with one or more substituents independently selected from CN, NO₂, halo, C₁₋₆alkyl, C₁₋₆fluoroalkyl, ═O, OR⁵, SR⁵ and NR⁵R⁶;
R² is absent or selected from H, CN, NO₂, halo, C₁₋₆alkyl, C₁₋₆fluoroalkyl, OR⁷, SR⁷ and NR⁷R⁸, and when present R² is ortho to 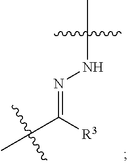
R³ is from H, C₁₋₄alkyl and C₁₋₄fluoroalkyl; or
R² and R³ are joined to form, together with the atoms therebetween, a 4 to 6 membered saturated or unsaturated ring, optionally containing one additional heteroatom selected from O, N and S, and optionally substituted with one or more substituents selected from C₁₋₆alkyl and C₁₋₆fluoroalkyl; and
X is selected from O, S and NR⁹;
R⁵, R⁶, R⁷, R⁸, and R⁹ are independently selected from H, C₁₋₆alkyl and C₁₋₆fluoroalkyl;
L¹ and L² are independently a linker moiety;
R¹⁵ is an antibody;
R¹⁶ is a drug; and
m is an integer from 1 to 20.

36. The antibody-drug conjugate of claim 36, wherein the antibody specifically binds to a receptor encoded by an ErbB gene.

37. The antibody-drug conjugate of claim 37, wherein the antibody specifically binds to an ErbB receptor selected from EGFR, HER2, HER3 and HER4.

38. The antibody-drug conjugate of claim 37, wherein the antibody specifically binds to the EGFR receptor.

39. The antibody-drug conjugate of any one of claims 35 to 38, wherein m is an integer from 1-10.

40. The antibody-drug conjugate of claim 35, wherein the compound is selected from 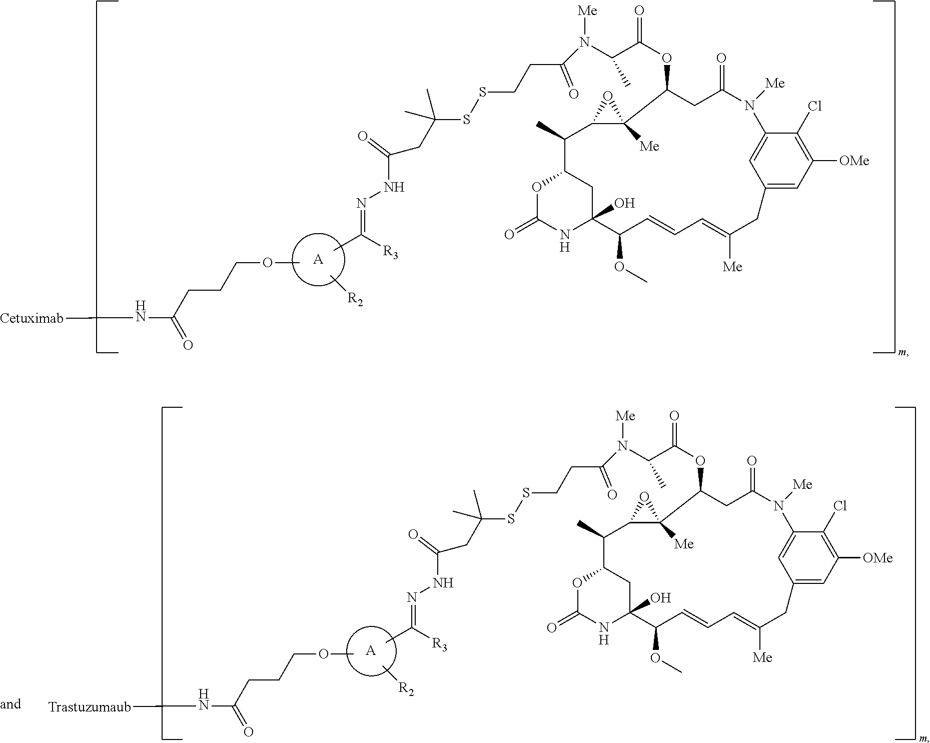
or a pharmaceutically acceptable salt and/or solvate thereof.

41. The antibody-drug conjugate of claim 35 selected from: 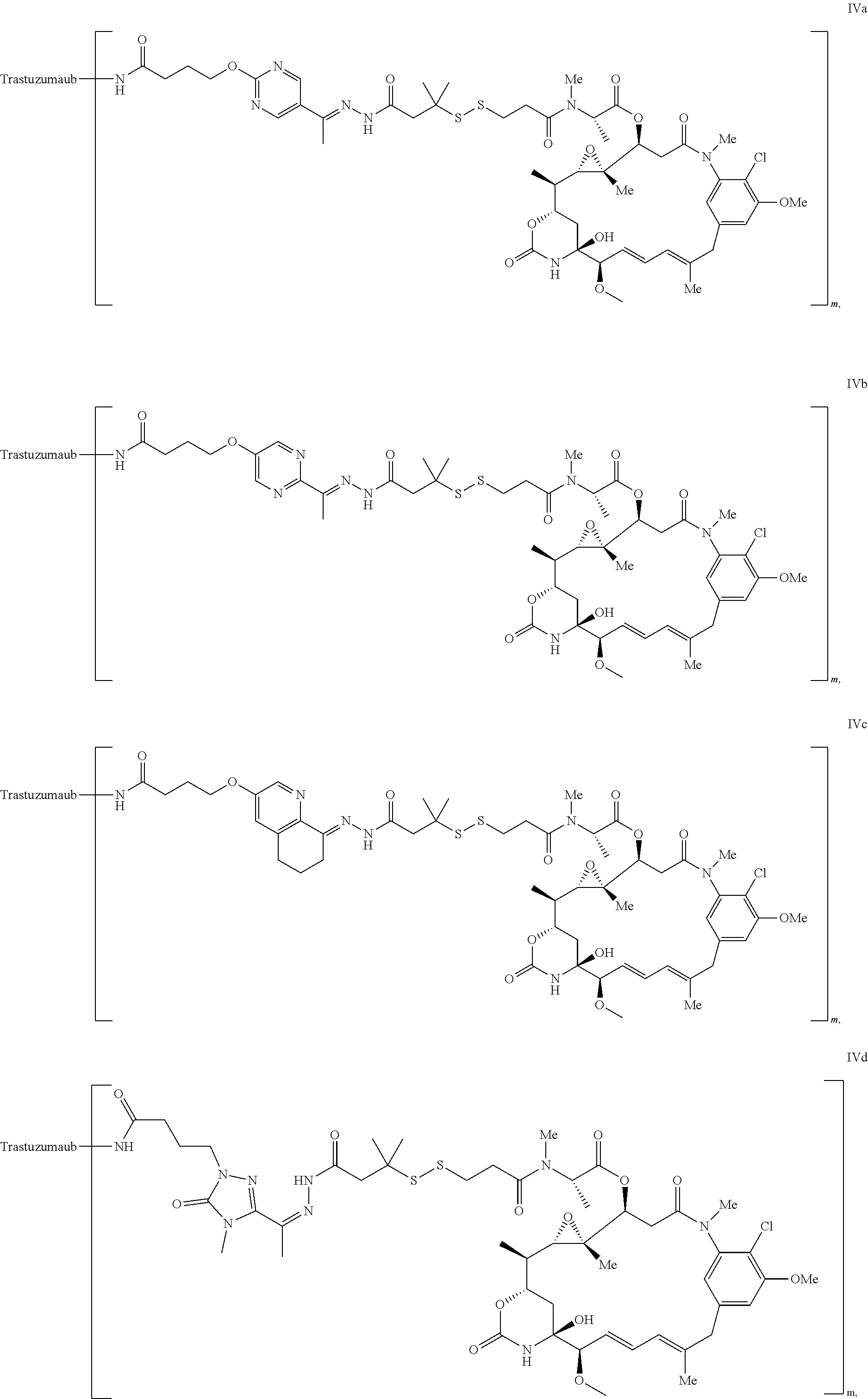 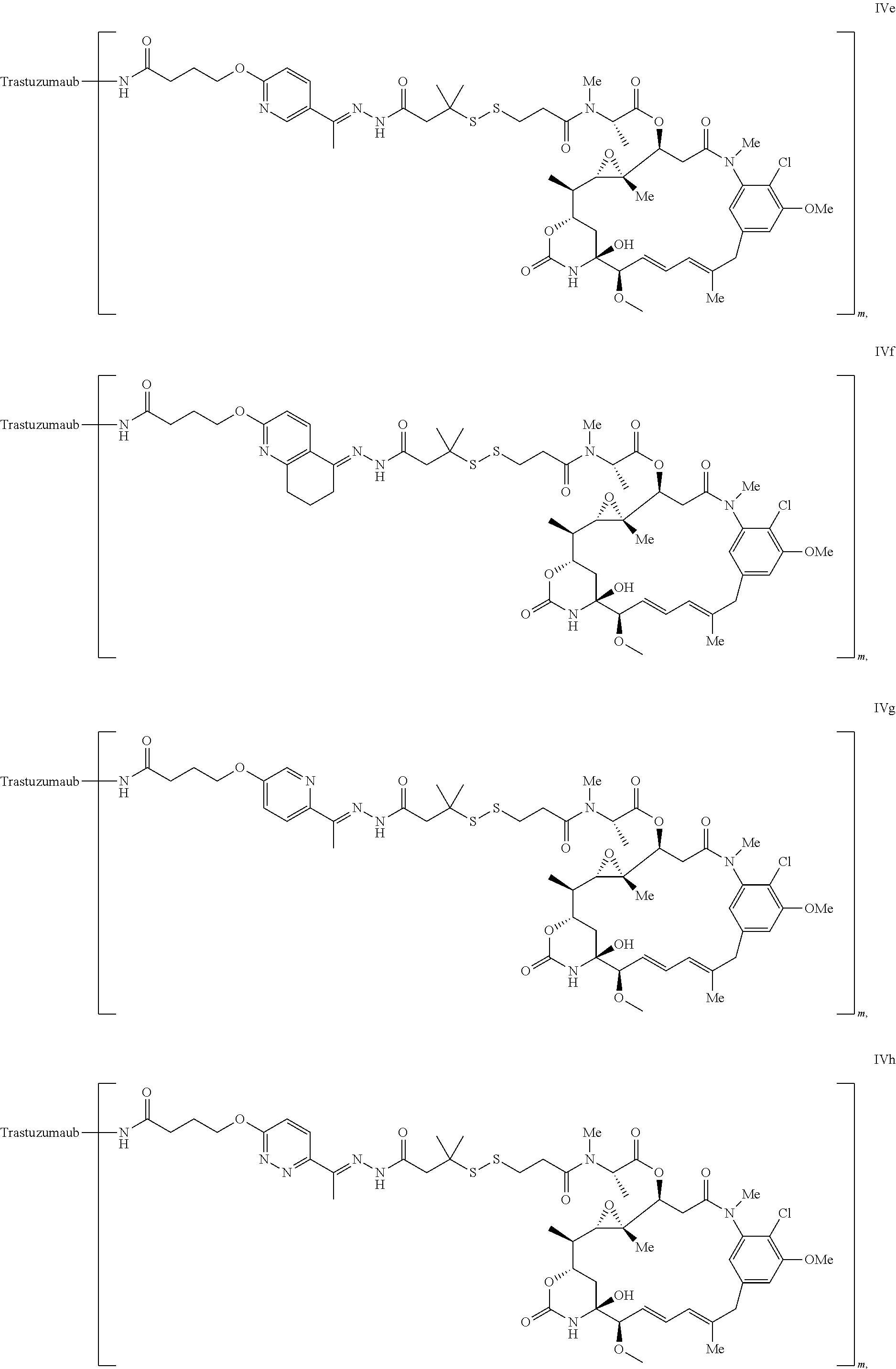 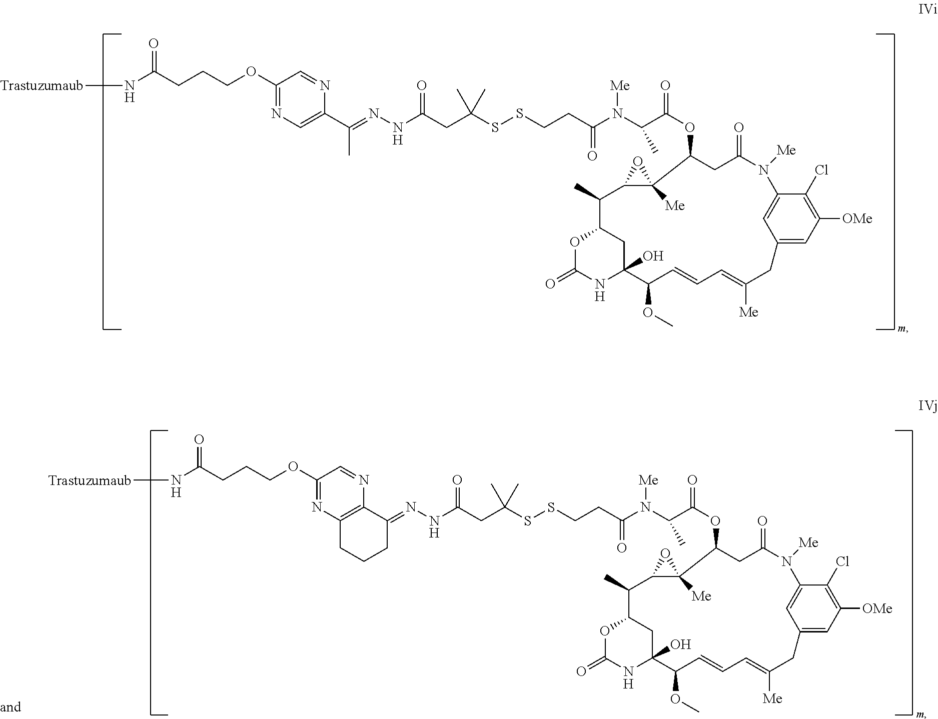
wherein m is from 1 to 10,
or a pharmaceutically acceptable salt and/or solvate thereof.

42. A compound of Formula (III): 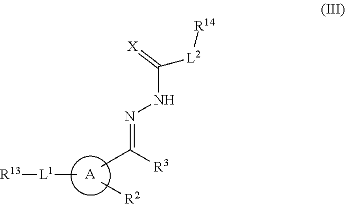
or a pharmaceutically acceptable salt and/or solvate thereof,
wherein
Ring A is a 5 or 6 membered unsaturated heterocycloalkyl or 5 or 6 membered heteraromatic ring each comprising 1 to 4 heteroatoms selected from O, N and S, and Ring A is optionally substituted with one or more substituents independently selected from CN, NO₂, halo, C₁₋₆alkyl, C₁₋₆fluoroalkyl, ═O, OR⁵, SR⁵ and NR⁵R⁶;
R² is absent or selected from H, CN, NO₂, halo, C₁₋₆alkyl, C₁₋₆fluoroalkyl, OR⁷, SR⁷ and NR⁷R⁸, and when present R² is ortho to 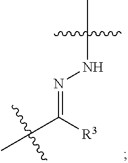
R³ is from H, C₁₋₄alkyl and C₁₋₄fluoroalkyl; or
R² and R³ are joined to form, together with the atoms therebetween, a 4 to 6 membered saturated or unsaturated ring, optionally containing one additional heteroatom selected from O, N and S, and optionally substituted with one or more substituents selected from C₁₋₆alkyl and C₁₋₆fluoroalkyl; and
X is selected from 0, S and NR⁹;
R⁵, R⁶, R⁷, R⁸, and R⁹ are independently selected from H, C₁₋₆alkyl and C₁₋₆fluoroalkyl;
L¹ and L² are independently a linker moiety; and
one of R¹³ and R¹⁴ is a reactive functional group; and the other of R¹³ and R¹⁴ is a compound to be linked to another same or different compound.

43. The compound of claim 42, wherein the compound is selected from 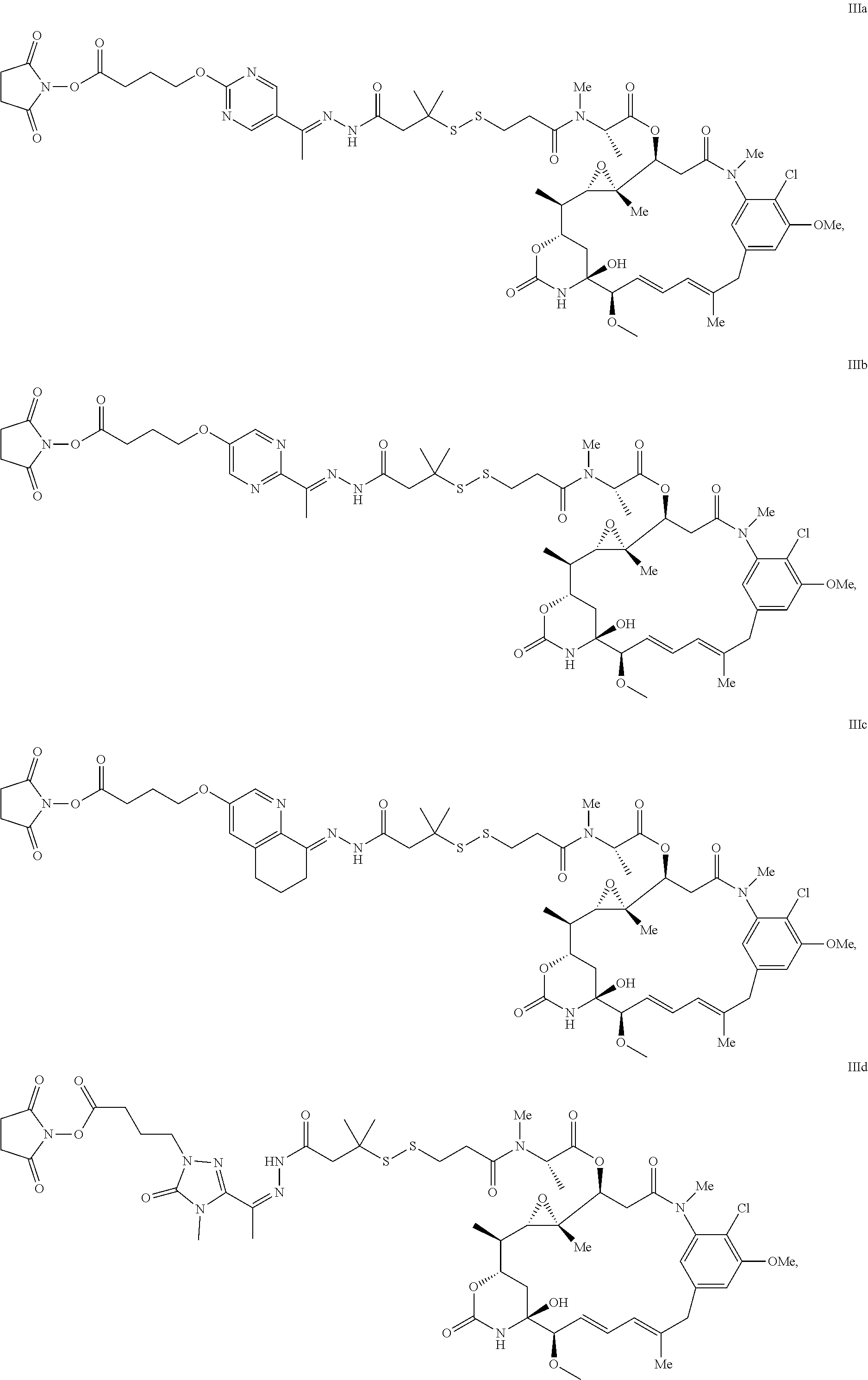 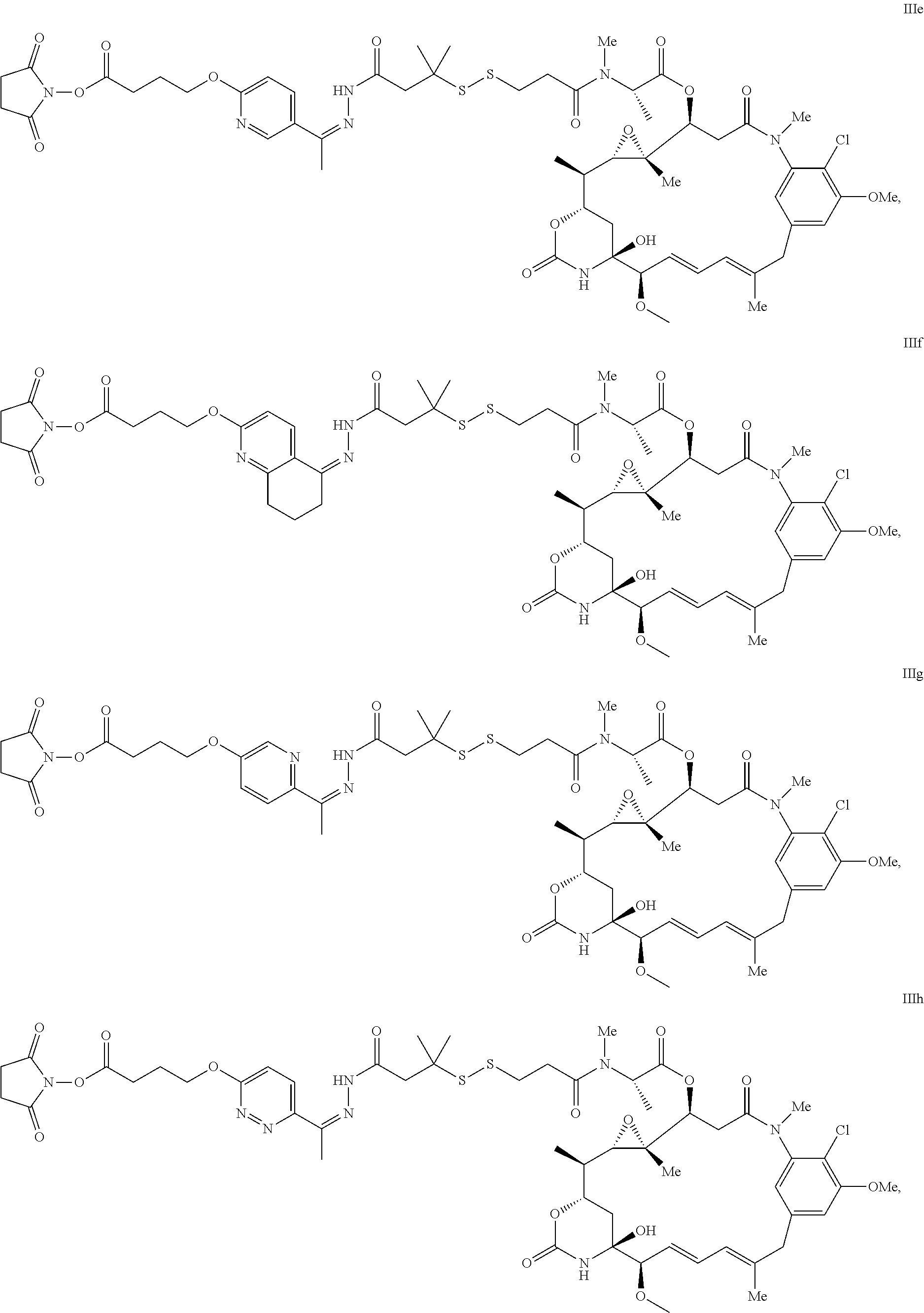 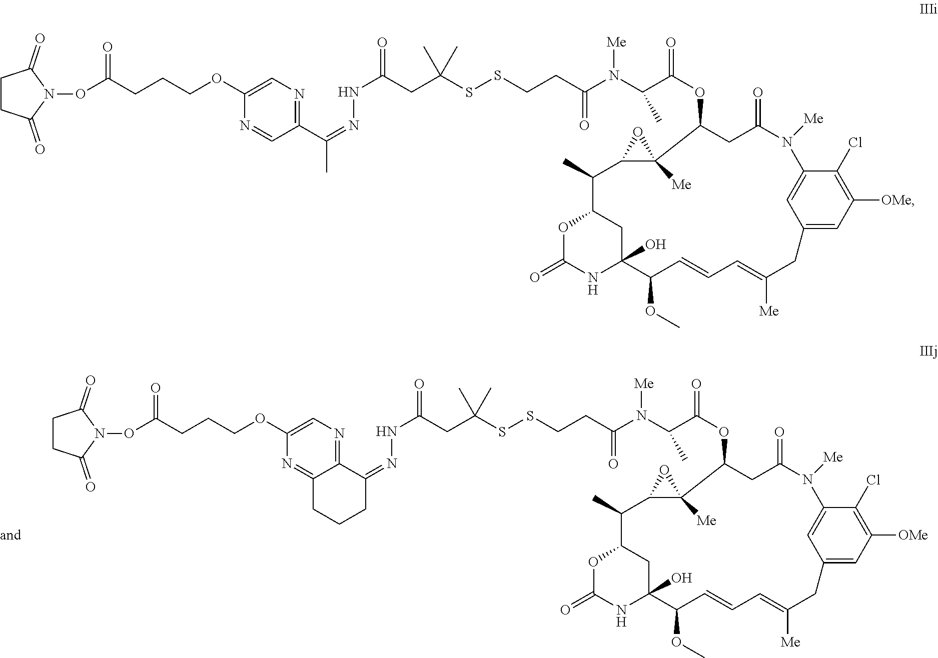 [SMILES: COc1cc2cc(c1Cl)N(C)C(=O)C[C@H](OC(=O)[C@H](C)N(C)C(=O)CCSSC(C)(C)CC(=O)N/N=C(/C)c1cnc(OCCCC(=O)ON3C(=O)CCC3=O)cn1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)CCSSC(C)(C)CC(=O)N/N=C1/CCCc3nc(OCCCC(=O)ON4C(=O)CCC4=O)cnc31)[C@]1(C)O[C@H]1[C@H](C)[C@@H]1C[C@@](O)(NC(=O)O1)[C@H](OC)/C=C/C=C(\C)C2]
or a pharmaceutically acceptable salt and/or solvate thereof.

44. A pharmaceutical composition comprising one or more compounds of Formula (II) of any one of claims 20 to 34 or a pharmaceutically acceptable salt and/or solvate thereof, and a pharmaceutically acceptable carrier and/or diluent.

45. A pharmaceutical composition comprising one or more compounds of Formula (IV) of any one of claims 35 to 41, or a pharmaceutically acceptable salt and/or solvate thereof, and a pharmaceutically acceptable carrier and/or diluent.

46. A method of treating and/or diagnosing one or more diseases, disorders or conditions comprising administering an effective amount of one or more compounds of Formula (II) of any one of claims 32 to 34 or a pharmaceutically acceptable salt and/or solvate thereof, and/or one or more compounds of Formula (IV) of any one of claims 35 to 41 or a pharmaceutically acceptable salt and/or solvate thereof, to a subject in need thereof.

47. The method of claim 46, wherein the disease, disorder or condition is a neoplastic disorder.

48. The method of claim 47, wherein the neoplastic disorder is cancer.

49. The method of claim 48, wherein the cancer is selected from breast cancer, skin cancer, prostate cancer, head and neck cancer, colorectal cancer, pancreatic cancer, kidney cancer, lung cancer and brain cancer.

50. A method of preparing an ADC of Formula (IV) as defined in claim 35 comprising:
(a) reacting a compound of Formula (I) as defined in any one of claims 1-31 with a drug to provide a Formula (I)-drug conjugate;
(b) reacting the Formula (I)-drug conjugate with an antibody to provide the ADC of Formula (IV); and optionally
(c) purifying the ADC of Formula (IV).

51. The method of claim 50, wherein the drug is an anticancer drug.

52. A method of preparing an ADC of Formula (IV) as defined in claim 35 comprising:
(a) reacting a compound of Formula (III) as defined in claim 42 or 43 with an antibody to provide the ADC of Formula (IV); and optionally
(b) purifying the ADC of Formula (IV).
